SEQUENCE LISTING (557 sequences)

SEQ ID NO: 1 - DNA, 141 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 130 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 3 - DNA, 1923 bp, Artificial Sequence
  Description of Artificial Sequence: Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 4 - DNA, 1272 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 5 - DNA, 547 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 6 - DNA, 1179 bp, Artificial Sequence
  Description of Artificial Sequence: Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 7 - DNA, 8 bp, Artificial Sequence
  Description of Artificial Sequence: Synthetic oligonucleotide
gtttaaac

SEQ ID NO: 8 - DNA, 581 bp, Artificial Sequence
  Description of Artificial Sequence: Synthetic polynucleotide
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 9 - DNA, 225 bp, Artificial Sequence
  Description of Artificial Sequence: Synthetic polynucleotide
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 10 - DNA, 213 bp, Artificial Sequence
  Description of Artificial Sequence: Synthetic polynucleotide
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 11 - skipped

SEQ ID NO: 12 - DNA, 1932 bp, Adeno-associated virus - 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 13 - DNA, 1876 bp, Artificial Sequence
  Description of Artificial Sequence: Synthetic polynucleotide
cgcagccaccatggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 14 - DNA, 1194 bp, Artificial Sequence
  Description of Artificial Sequence: Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 15 - DNA, 141 bp, Artificial Sequence
  Description of Artificial Sequence: Synthetic polynucleotide
aataaacgataacgccgttggtggcgtgaggcatgtaaaaggttacatcattatcttgttcgccatccggttggtataaatagacgttcatgttggtttttgtttcagttgcaagttggctgcggcgcgcgcagcaccttt

SEQ ID NO: 16-17 - skipped (2 entries)

SEQ ID NO: 18 - DNA, 241 bp, Artificial Sequence
  Description of Artificial Sequence: Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 19 - DNA, 215 bp, Artificial Sequence
  Description of Artificial Sequence: Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccac

SEQ ID NO: 20 - skipped

SEQ ID NO: 21 - DNA, 546 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 22 - DNA, 317 bp, Artificial Sequence
  Description of Artificial Sequence: Synthetic polynucleotide
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 23 - DNA, 576 bp, Artificial Sequence
  Description of Artificial Sequence: Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 24 - DNA, 1313 bp, Artificial Sequence
  Description of Artificial Sequence: Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaa

SEQ ID NO: 25 - protein, 19 aa, Artificial Sequence
  Description of Artificial Sequence: Synthetic peptide
MDWTWRILFLVAAATGAHS

SEQ ID NO: 26 - protein, 19 aa, Artificial Sequence
  Description of Artificial Sequence: Synthetic peptide
MLPSQLIGFLLLWVPASRG

SEQ ID NO: 27 - protein, 7 aa, Artificial Sequence
  Description of Artificial Sequence: Synthetic peptide
PKKKRKV

SEQ ID NO: 28-44 - skipped (17 entries)

SEQ ID NO: 45 - DNA, 6 bp, Artificial Sequence
  Description of Artificial Sequence: Synthetic oligonucleotide
ggttga

SEQ ID NO: 46 - DNA, 4 bp, Artificial Sequence
  Description of Artificial Sequence: Synthetic oligonucleotide
agtt

SEQ ID NO: 47 - DNA, 6 bp, Artificial Sequence
  Description of Artificial Sequence: Synthetic oligonucleotide
ggttgg

SEQ ID NO: 48 - DNA, 6 bp, Artificial Sequence
  Description of Artificial Sequence: Synthetic oligonucleotide
agttgg

SEQ ID NO: 49 - DNA, 6 bp, Artificial Sequence
  Description of Artificial Sequence: Synthetic oligonucleotide
agttga

SEQ ID NO: 50 - DNA, 6 bp, Artificial Sequence
  Description of Artificial Sequence: Synthetic oligonucleotide
rrttrr

SEQ ID NO: 51 - DNA, 141 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 52 - DNA, 130 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 53-65 - skipped (13 entries)

SEQ ID NO: 66 - DNA, 141 bp, Artificial Sequence; identical to SEQ ID NO: 15
  Description of Artificial Sequence: Synthetic polynucleotide

SEQ ID NO: 67 - DNA, 1876 bp, Artificial Sequence
  Description of Artificial Sequence: Synthetic polynucleotide
cgcagccaccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 68 - DNA, 129 bp, Artificial Sequence
  Description of Artificial Sequence: Synthetic polynucleotide
atcatggagataattaaaatgataaccatctcgcaaataaataagtattttactgttttcgtaacagttttgtaataaaaaaacctataaatattccggattattcataccgtcccaccatcgggcgcg

SEQ ID NO: 69 - DNA, 1203 bp, Artificial Sequence
  Description of Artificial Sequence: Synthetic polynucleotide
gccgccaccatggagttggtgggctggctcgtggacaaaggcattacttcggaaaagcagtggattcaggaggatcaggcatcttacatctcattcaacgctgccagtaactcgaggtcccagatcaaggcagcgctggacaacgcgggaaagattatgagtctgaccaaaactgctccagactacctcgttggtcagcaaccggtggaagatatctccagcaacaggatctacaagattctggagctcaacggctacgaccctcaatacgctgcctcagtgttcttgggttgggccaccaagaaattcggcaagagaaacactatctggctgttcggccccgctaccactggaaagacaaacatcgcagaagcgattgctcacacggtgccattctacggctgcgtcaactggacaaacgagaacttcccgttcaacgactgtgtcgataagatggttatctggtgggaggaaggaaagatgacggccaaagtggtcgaaagcgccaaggcaattctgggtggctctaaagtgcgcgtcgaccagaagtgcaaatcttcagctcaaatcgatcctacccccgttattgtgacatcaaacacgaacatgtgtgccgtgatcgacggaaacagtacaacgttcgaacaccagcaacctctccaggatcgtatgttcaagttcgagctcacccgccgtttggaccatgatttcggcaaggtcactaaacaagaggttaaggacttcttccgctgggctaaagatcacgttgtggaggttgaacatgagttctacgtcaagaaaggaggtgctaagaaacgtccagccccgtcggacgcagatatctccgaacctaagagggtgagagagtcggtcgcacagccaagcacttctgacgcagaagcttccattaactacgcagataggtaccaaaacaagtgcagcagacacgtgggtatgaacttgatgctgttcccatgccgccagtgtgagcgtatgaaccaaaactctaacatctgtttcacacatggccagaaggactgcctcgaatgtttccctgtgtcagagagtcagcccgtctcagtcgttaagaaagcttaccaaaagttgtgctacatccaccatattatgggtaaagtccctgatgcctgtaccgcttgtgatctggtcaacgtggatttggacgactgtattttcgagcaataa

SEQ ID NO: 70-73 - skipped (4 entries)

SEQ ID NO: 74 - DNA, 1177 bp, Artificial Sequence
  Description of Artificial Sequence: Synthetic polynucleotide
ggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 75-100 - skipped (26 entries)

SEQ ID NO: 101 - DNA, 70 bp, Artificial Sequence
  Description of Artificial Sequence: Synthetic oligonucleotide
gcgcgctcgctcgctcactgaggccgcccgggaaacccgggcgtgcgcctcagtgagcgagcgagcgcgc

SEQ ID NO: 102 - DNA, 70 bp, Artificial Sequence
  Description of Artificial Sequence: Synthetic oligonucleotide
gcgcgctcgctcgctcactgaggcgcacgcccgggtttcccgggcggcctcagtgagcgagcgagcgcgc

SEQ ID NO: 103 - DNA, 72 bp, Artificial Sequence
  Description of Artificial Sequence: Synthetic oligonucleotide
gcgcgctcgctcgctcactgaggccgtcgggcgacctttggtcgcccggcctcagtgagcgagcgagcgcgc

SEQ ID NO: 104 - DNA, 72 bp, Artificial Sequence
  Description of Artificial Sequence: Synthetic oligonucleotide
gcgcgctcgctcgctcactgaggccgggcgaccaaaggtcgcccgacggcctcagtgagcgagcgagcgcgc

SEQ ID NO: 105 - DNA, 72 bp, Artificial Sequence
  Description of Artificial Sequence: Synthetic oligonucleotide
gcgcgctcgctcgctcactgaggccgcccgggcaaagcccgggcgtcggcctcagtgagcgagcgagcgcgc

SEQ ID NO: 106 - DNA, 72 bp, Artificial Sequence
  Description of Artificial Sequence: Synthetic oligonucleotide
gcgcgctcgctcgctcactgaggccgacgcccgggctttgcccgggcggcctcagtgagcgagcgagcgcgc

SEQ ID NO: 107 - DNA, 83 bp, Artificial Sequence
  Description of Artificial Sequence: Synthetic oligonucleotide
gcgcgctcgctcgctcactgaggccgcccgggcaaagcccgggcgtcgggctttgcccggcctcagtgagcgagcgagcgcgc

SEQ ID NO: 108 - DNA, 83 bp, Artificial Sequence
  Description of Artificial Sequence: Synthetic oligonucleotide
gcgcgctcgctcgctcactgaggccgggcaaagcccgacgcccgggctttgcccgggcggcctcagtgagcgagcgagcgcgc

SEQ ID NO: 109 - DNA, 77 bp, Artificial Sequence
  Description of Artificial Sequence: Synthetic oligonucleotide
gcgcgctcgctcgctcactgaggccgaaacgtcgggcgacctttggtcgcccggcctcagtgagcgagcgagcgcgc

SEQ ID NO: 110 - DNA, 77 bp, Artificial Sequence
  Description of Artificial Sequence: Synthetic oligonucleotide
gcgcgctcgctcgctcactgaggccgggcgaccaaaggtcgcccgacgtttcggcctcagtgagcgagcgagcgcgc

SEQ ID NO: 111 - DNA, 51 bp, Artificial Sequence
  Description of Artificial Sequence: Synthetic oligonucleotide
gcgcgctcgctcgctcactgaggcaaagcctcagtgagcgagcgagcgcgc

SEQ ID NO: 112 - DNA, 51 bp, Artificial Sequence
  Description of Artificial Sequence: Synthetic oligonucleotide
gcgcgctcgctcgctcactgaggctttgcctcagtgagcgagcgagcgcgc

SEQ ID NO: 113 - DNA, 80 bp, Artificial Sequence
  Description of Artificial Sequence: Synthetic oligonucleotide
gcgcgctcgctcgctcactgaggccgcccgggcgtcgggcgacctttggtcgcccggcctcagtgagcgagcgagcgcgc

SEQ ID NO: 114 - DNA, 80 bp, Artificial Sequence
  Description of Artificial Sequence: Synthetic oligonucleotide
gcgcgctcgctcgctcactgaggccgggcgaccaaaggtcgcccgacgcccgggcggcctcagtgagcgagcgagcgcgc

SEQ ID NO: 115 - DNA, 77 bp, Artificial Sequence
  Description of Artificial Sequence: Synthetic oligonucleotide
gcgcgctcgctcgctcactgaggcgcccgggcgtcgggcgacctttggtcgcccggcctcagtgagcgagcgagcgc

SEQ ID NO: 116 - DNA, 79 bp, Artificial Sequence
  Description of Artificial Sequence: Synthetic oligonucleotide
gcgcgctcgctcgctcactgaggccgggcgaccaaaggtcgcccgacgcccgggcgcctcagtgagcgagcgagcgcgc

SEQ ID NO: 117-499 - skipped (383 entries)

SEQ ID NO: 500 - DNA, 43 bp, Artificial Sequence
  Description of Artificial Sequence: Synthetic oligonucleotide
gcccgctggtttccagcgggctgcgggcccgaaacgggcccgc

SEQ ID NO: 501 - DNA, 28 bp, Artificial Sequence
  Description of Artificial Sequence: Synthetic oligonucleotide
cgggcccgtgcgggcccaaagggcccgc

SEQ ID NO: 502 - DNA, 28 bp, Artificial Sequence
  Description of Artificial Sequence: Synthetic oligonucleotide
gcccgggcacgcccgggtttcccgggcg

SEQ ID NO: 503 - DNA, 22 bp, Artificial Sequence
  Description of Artificial Sequence: Synthetic oligonucleotide
cgtgcgggcccaaagggcccgc

SEQ ID NO: 504 - DNA, 21 bp, Artificial Sequence
  Description of Artificial Sequence: Synthetic oligonucleotide
cgggcgaccaaaggtcgcccg

SEQ ID NO: 505 - DNA, 20 bp, Artificial Sequence
  Description of Artificial Sequence: Synthetic oligonucleotide
cgcccgggctttgcccgggc

SEQ ID NO: 506 - DNA, 42 bp, Artificial Sequence
  Description of Artificial Sequence: Synthetic oligonucleotide
cgggcgaccaaaggtcgcccgacgcccgggctttgcccgggc

SEQ ID NO: 507 - DNA, 21 bp, Artificial Sequence
  Description of Artificial Sequence: Synthetic oligonucleotide
cgggcgaccaaaggtcgcccg

SEQ ID NO: 508 - DNA, 20 bp, Artificial Sequence
  Description of Artificial Sequence: Synthetic oligonucleotide
cgcccgggctttgcccgggc

SEQ ID NO: 509 - DNA, 34 bp, Artificial Sequence
  Description of Artificial Sequence: Synthetic oligonucleotide
cgggcgaccaaaggtcgcccgacgcccgggcggc

SEQ ID NO: 510 - DNA, 21 bp, Artificial Sequence
  Description of Artificial Sequence: Synthetic oligonucleotide
cgggcgaccaaaggtcgcccg

SEQ ID NO: 511 - DNA, 20 bp, Artificial Sequence
  Description of Artificial Sequence: Synthetic oligonucleotide
cgcccgggctttgcccgggc

SEQ ID NO: 512 - DNA, 30 bp, Artificial Sequence
  Description of Artificial Sequence: Synthetic oligonucleotide
cggggcccgacgcccgggctttgcccgggc

SEQ ID NO: 513 - DNA, 21 bp, Artificial Sequence
  Description of Artificial Sequence: Synthetic oligonucleotide
cgggcgaccaaaggtcgcccg

SEQ ID NO: 514 - DNA, 20 bp, Artificial Sequence
  Description of Artificial Sequence: Synthetic oligonucleotide
cgcccgggctttgcccgggc

SEQ ID NO: 515 - DNA, 29 bp, Artificial Sequence
  Description of Artificial Sequence: Synthetic oligonucleotide
cgggcccgacgcccgggctttgcccgggc

SEQ ID NO: 516 - DNA, 21 bp, Artificial Sequence
  Description of Artificial Sequence: Synthetic oligonucleotide
cgggcgaccaaaggtcgcccg

SEQ ID NO: 517 - DNA, 20 bp, Artificial Sequence
  Description of Artificial Sequence: Synthetic oligonucleotide
cgcccgggctttgcccgggc

SEQ ID NO: 518 - DNA, 20 bp, Artificial Sequence
  Description of Artificial Sequence: Synthetic oligonucleotide
gcccgggcaaagcccgggcg

SEQ ID NO: 519 - DNA, 21 bp, Artificial Sequence
  Description of Artificial Sequence: Synthetic oligonucleotide
cgggcgacctttggtcgcccg

SEQ ID NO: 520 - DNA, 42 bp, Artificial Sequence
  Description of Artificial Sequence: Synthetic oligonucleotide
gcccgggcaaagcccgggcgtcgggcgacctttggtcgcccg

SEQ ID NO: 521 - DNA, 20 bp, Artificial Sequence
  Description of Artificial Sequence: Synthetic oligonucleotide
gcccgggcaaagcccgggcg

SEQ ID NO: 522 - DNA, 31 bp, Artificial Sequence
  Description of Artificial Sequence: Synthetic oligonucleotide
gcccgggcgtcgggcgacctttggtcgcccg

SEQ ID NO: 523 - DNA, 20 bp, Artificial Sequence
  Description of Artificial Sequence: Synthetic oligonucleotide
gcccgggcaaagcccgggcg

SEQ ID NO: 524 - DNA, 21 bp, Artificial Sequence
  Description of Artificial Sequence: Synthetic oligonucleotide
cgggcgacctttggtcgcccg

SEQ ID NO: 525 - DNA, 34 bp, Artificial Sequence
  Description of Artificial Sequence: Synthetic oligonucleotide
gccgcccgggcgacgggcgacctttggtcgcccg

SEQ ID NO: 526 - DNA, 20 bp, Artificial Sequence
  Description of Artificial Sequence: Synthetic oligonucleotide
gcccgggcaaagcccgggcg

SEQ ID NO: 527 - DNA, 21 bp, Artificial Sequence
  Description of Artificial Sequence: Synthetic oligonucleotide
cgggcgacctttggtcgcccg

SEQ ID NO: 528 - DNA, 31 bp, Artificial Sequence; identical to SEQ ID NO: 522
  Description of Artificial Sequence: Synthetic oligonucleotide

SEQ ID NO: 529 - DNA, 21 bp, Artificial Sequence
  Description of Artificial Sequence: Synthetic oligonucleotide
cgggcgacctttggtcgcccg

SEQ ID NO: 530 - skipped

SEQ ID NO: 531 - DNA, 16 bp, Artificial Sequence
  Description of Artificial Sequence: Synthetic oligonucleotide
gcgcgctcgctcgctc

SEQ ID NO: 532-539 - skipped (8 entries)

SEQ ID NO: 540 - DNA, 91 bp, Artificial Sequence
  Description of Artificial Sequence: Synthetic oligonucleotide
gcgcgctcgctcgctcactgaggccgcccgggcaaagcccgggcgtcgggcgacctttggtcgcccggcctcagtgagcgagcgagcgcgc

SEQ ID NO: 541 - DNA, 91 bp, Artificial Sequence
  Description of Artificial Sequence: Synthetic oligonucleotide
gcgcgctcgctcgctcactgaggccgggcgaccaaaggtcgcccgacgcccgggctttgcccgggcggcctcagtgagcgagcgagcgcgc

SEQ ID NO: 542 - DNA, 8 bp, Artificial Sequence
  Description of Artificial Sequence: Synthetic oligonucleotide
ttaattaa

SEQ ID NO: 543-547 - skipped (5 entries)

SEQ ID NO: 548 - DNA, 42 bp, Artificial Sequence; identical to SEQ ID NO: 506
  Description of Artificial Sequence: Synthetic oligonucleotide

SEQ ID NO: 549 - DNA, 42 bp, Artificial Sequence; identical to SEQ ID NO: 520
  Description of Artificial Sequence: Synthetic oligonucleotide

SEQ ID NO: 550 - skipped

SEQ ID NO: 551 - DNA, 43 bp, Artificial Sequence; identical to SEQ ID NO: 500
  Description of Artificial Sequence: Synthetic oligonucleotide

SEQ ID NO: 552 - DNA, 28 bp, Artificial Sequence
  Description of Artificial Sequence: Synthetic oligonucleotide
cgggcccgtgcgggcccaaagggcccgc

SEQ ID NO: 553 - DNA, 28 bp, Artificial Sequence
  Description of Artificial Sequence: Synthetic oligonucleotide
gcccgggcacgcccgggtttcccgggcg

SEQ ID NO: 554 - DNA, 22 bp, Artificial Sequence
  Description of Artificial Sequence: Synthetic oligonucleotide
cgtgcgggcccaaagggcccgc

SEQ ID NO: 555 - DNA, 43 bp, Artificial Sequence
  Description of Artificial Sequence: Synthetic oligonucleotide
gcgggccggaaacgggcccgctgcccgctggtttccagcgggc

SEQ ID NO: 556 - DNA, 28 bp, Artificial Sequence
  Description of Artificial Sequence: Synthetic oligonucleotide
cgcccgggaaacccgggcgtgcccgggc

SEQ ID NO: 557 - DNA, 28 bp, Artificial Sequence
  Description of Artificial Sequence: Synthetic oligonucleotide
gggccgcccgggaaacccgggcgtgccc

(19) United States
(12) Patent Application Publication
(10) Pub. No.: US 2021/0059953 A1
(43) Pub. Date: Mar. 4, 2021

(54) LIPID NANOPARTICLE FORMULATIONS OF NON-VIRAL, CAPSID-FREE DNA VECTORS

(71) Applicant: GENERATION BIO CO., Cambridge, MA (US)

(72) Inventors: Robert Michael Kotin, Cambridge, MA (US); Ozan Alkan, Cambridge, MA (US); Douglas Anthony Kerr, Cambridge, MA (US); Ara Karl Malakian, Cambridge, MA (US); Matthew John Simmons, Cambridge, MA (US); Matthew G. Stanton, Cambridge, MA (US); Jie Su, Cambridge, MA (US); Teresa L. Wright, Cambridge, MA (US)

(21) Appl. No.: 16/644,574

(22) PCT Filed: Sep. 7, 2018
(86) PCT No.: PCT/US18/50042

§ 371 Date: Mar. 5, 2020

Related U.S. Application Data
(60) Provisional application No. 62/556,334, filed on Sep. 8, 2017.
(60) Provisional application No. 62/556,333, filed on Sep. 8, 2017.
(60) Provisional application No. 62/556,381, filed on Sep. 9, 2017.
(60) Provisional application No. 62/675,324, filed on May 23, 2018.
(60) Provisional application No. 62/675,322, filed on May 23, 2018.
(60) Provisional application No. 62/675,317, filed on May 23, 2018.
(60) Provisional application No. 62/675,327, filed on May 23, 2018.

(51) Int. Cl. A61K 9/51; C12N 15/86; A61K 9/127

(52) U.S. Cl. CPC A61K 9/5176; C12N 15/86; C12N 2710/14143; C12N 2710/14144; A61K 9/127

(57) ABSTRACT

Provided herein are lipid nanoparticle formulations that comprise an ionizable lipid and non-viral, capsid-free DNA vectors with covalently-closed ends.

### CROSS-REFERENCE TO RELATED APPLICATIONS

The application claims benefit under 35 U.S.C. § 119(e) of U.S. Provisional Application No. 62/556,334, filed Sep. 8, 2017, No. 62/556,333, filed Sep. 8, 2017, No. 62/556,381, filed Sep. 9, 2017, No. 62/675,317, filed May 23, 2018, No. 62/675,322, filed May 23, 2018, No. 62/675,324, filed May 23, 2018, and No. 62/675,327, filed May 23, 2018, the content of each of which is incorporated herein by reference in its entirety.

### SEQUENCE LISTIING

The instant application contains a Sequence Listing which has been submitted electronically in ASCII format and is hereby incorporated by reference in its entirety. Said ASCII copy, created on Sep. 7, 2018, is named 080170-090660WOPT SL.txt and is 63,790 bytes in size.

### TECHNICAL FIELD

The present invention is directed to lipid nanoparticle (LNP) formulations of non-viral, capsid-free DNA vectors and their use for the delivery of exogenous DNA sequences to a target cell, tissue, organ or organism.

### BACKGROUND

Recently, non-viral, capsid-free DNA vectors with covalently-closed ends that contain transgenes flanked by AAV 2 ITRs were reported. However, targeted delivery of these DNA vectors to cells, in vitro and in vivo, remains challenging. Accordingly, there remains a need in the art for formulations that address these challenges.

### SUMMARY

In one aspect, provided herein are novel lipid formulations comprising an ionizable lipid and a capsid free, non-viral vector (ceDNA). The ceDNA vectors described herein are capsid-free, linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence that are different, or asymmetric with respect to each other. In one aspect, non-viral capsid-free DNA vectors with covalently-closed ends are preferably linear duplex molecules, and are obtainable from a vector polynucleotide that encodes a heterologous nucleic acid operatively positioned between two different inverted terminal repeat sequences (ITRs) (e.g. AAV ITRs), wherein at least one of the ITRs comprises a terminal resolution site and a replication protein binding site (RPS) (sometimes referred to as a replicative protein binding site), e.g. a Rep binding site, and one of the ITRs comprises a deletion, insertion, and/or substitution with respect to the other ITR. That is, one of the ITRs is asymmetrical relative to the other ITR. In one embodiment, at least one of the ITRs is an AAV ITR, e.g. a wild type AAV ITR or modified AAV ITR. In one embodiment, at least one of the ITRs is a modified ITR relative to the other ITR—that is, the ceDNA comprises ITRs that are asymmetric relative to each other.

In one embodiment, at least one of the ITRs is a non-functional ITR.

In some embodiments, one or more of the ITRs is not a wild type ITR.

In some embodiments, the ceDNA vector when digested with a restriction enzyme having a single recognition site on the ceDNA vector and analyzed by both native and denaturing gel electrophoresis displays characteristic bands of linear and continuous DNA as compared to linear and non-continuous DNA controls.

In some embodiments, one or more of the asymmetric ITR sequences of the ceDNA vector are from a virus selected from a parvovirus, a dependovirus, and an adeno-associated virus (AAV). In some embodiments, the asymmetric ITRs are from different viral serotypes. For example, one or more asymmetric ITRs can be from an AAV serotype selected from AAV1, AAV2, AAV3, AAV4, AAV5, AAV6, AAV7, AAV8, AAV9, AAV10, AAV11, and AAV12.

In some embodiments, one or more of the asymmetric ITR sequences of the ceDNA vector are synthetic.

In some embodiments, at least one (e.g., one or both) of the asymmetric ITRs is modified by a deletion, insertion, and/or substitution in at least one of the ITR regions selected from A, A′, B, B′, C, C′, D, and D′.

In some embodiments, the ceDNA vector comprises at least two asymmetric ITRs selected from: (a) SEQ ID NO: 1 and SEQ ID NO:52; and (b) SEQ ID NO: 2 and SEQ ID NO: 51.

In some embodiments, the ceDNA vector is obtained from a process comprising the steps of: (a) incubating a population of insect cells harboring a ceDNA expression construct in the presence of at least one Rep protein, wherein the ceDNA expression construct encodes the ceDNA vector, under conditions effective and for a time sufficient to induce production of the ceDNA vector within the insect cells; and (b) isolating the ceDNA vector from the insect cells. Without limitations, the ceDNA expression construct is can be a ceDNA plasmid, a ceDNA bacmid, or a ceDNA baculovirus.

Generally, the insect cell insect cell expresses at least one Rep protein. The at least one Rep protein can be from a virus selected from a parvovirus, a dependovirus, and an adeno-associated virus (AAV). For example, the at least one Rep protein can be from an AAV serotype selected from AAV1, AAV2, AAV3, AAV4, AAV5, AAV6, AAV7, AAV8, AAV9, AAV10, AAV11, and AAV12.

In some embodiments the ionizable lipid is a lipid described in a publication listed in Table 1.

In some embodiments of the various aspects disclosed herein, the presence of ceDNA can be confirmed by digestion with a restriction enzyme having a single recognition site on the ceDNA vector and analyzing the digested DNA material on denaturing and non-denaturing gels to confirm the presence of characteristic bands of linear and continuous DNA as compared to linear and non-continuous DNA.

In some embodiments, the DNA vector is obtained from a vector polynucleotide, wherein the vector polynucleotide encodes a heterologous nucleic acid operatively positioned between two inverted terminal repeat sequences (ITRs), wherein the two ITS are different from each other (asymmetric), and at least one of the ITRs is a functional ITR comprising a functional terminal resolution site and a Rep binding site, and one of the ITRs comprises a deletion, insertion, or substitution relative to the functional ITR; the presence of Rep protein inducing replication of the vector polynucleotide.

In some embodiments, the production of the DNA vector in an insect cell. For example, the DNA vector being obtainable from a process comprising the steps of: (a) incubating a population of insect cells harboring the vector polynucleotide, which is devoid of viral capsid coding sequences, in the presence of Rep protein under conditions effective and for time sufficient to induce production of the capsid-free, non-viral DNA vector within the insect cells, wherein the insect cells do not comprise production of capsid-free, non-viral DNA within the insect cells in the absence of the vector; and (b) harvesting and isolating the capsid-free, non-viral DNA from the insect cells. In some further embodiments, the presence of the capsid-free, non-viral DNA isolated from the insect cells can be confirmed. For example, the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.

In some embodiments, the DNA vector is obtained from a vector polynucleotide. For example, the DNA vector is obtained from a vector polynucleotide encoding a heterologous nucleic acid operatively positioned between a first and a second AAV2 inverted terminal repeat DNA polynucleotide sequence (ITRs), with at least one of the ITRs having at least one polynucleotide deletion, insertion, or substitution with respect to the corresponding AAV2 wild type ITR of SEQ ID NO:1 or SEQ ID NO:51 to induce replication of the DNA vector in an insect cell in the presence of Rep protein. In some further embodiments of this, the DNA vector is obtainable from a process comprising the steps of: (a) incubating a population of insect cells harboring the vector polynucleotide, which is devoid of viral capsid coding sequences, in the presence of Rep protein, under conditions effective and for a time sufficient to induce production of the capsid-free, non-viral DNA within the insect cells, wherein the insect cells do not comprise viral capsid coding sequences; and (b) harvesting and isolating the capsid-free, non-viral DNA from the insect cells. In some further embodiments, the presence of the capsid-free, non-viral DNA isolated from the insect cells can be confirmed. For example, the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.

In some embodiments, the lipid nanoparticle can further comprise a non-cationic lipid, a PEG conjugated lipid, a sterol, or any combination thereof.

In some embodiments, the lipid nanoparticle further comprises a non-cationic lipid, wherein the non-ionic lipid is selected from the group consisting of distearoyl-sn-glycero-phosphoethanolamine, di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), di stearoyl-phosphatidyl-ethanolamine (DSPE), monomethyl-phosphatidylethanolamine (such as 16-0-monomethyl PE), dimethyl-phosphatidylethanolamine (such as 16-0-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOP 5), sphingomyelin (SM), dimyristoyl phosphatidylcholine (DMPC), dimyristoyl phosphatidylglycerol (DMPG), di stearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine and non-cationic lipids described, for example, in WO2017/099823 or US2018/0028664.

In some embodiments, the lipid particle further comprises a conjugated lipid, wherein the conjugated lipid, wherein the conjugated-lipid is selected from the group consisting of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-di stearoyl-sn-glycero-3 -phosphoethanolamine sodium salt, and those described in Table 2.

In some embodiments, the lipid particle further comprises cholesterol or a cholesterol derivative described in PCT publication WO2009/127060 or US patent publication US2010/0130588.

In some embodiments, the lipid particle comprises an ionizable lipid, a non-cationic lipid, a conjugated lipid that inhibits aggregation of particles, and a sterol. The amount of the ionizable lipid, the non-cationic lipid, the conjugated lipid that inhibits aggregation of particles, and the sterol can be varied independently. In some embodiments, the lipid nanoparticle comprises an ionizable lipid in an amount from about 20 mol % to about 90 mol % of the total lipid present in the particle, a non-cationic lipid in an amount from about 5 mol % to about 30 mol % of the total lipid present in the particle, a conjugated lipid that inhibits aggregation of particles in an amount from about 0.5 mol % to about 20 mol % of the total lipid present in the particle, and a sterol in an amount from about 20 mol % to about 50 mol % of the total lipid present in the particle.

The ratio of total lipid to DNA vector can be varied as desired. For example, the total lipid to DNA vector (mass or weight) ratio can be from about 10:1 to about 30:1.

Also provided herein is a composition comprising a first lipid nanoparticle and an additional compound. The first lipid nanoparticle comprises an ionizable lipid and a first capsid free, non-viral vector. The capsid free, non-viral vector when digested with a restriction enzyme having a single recognition site on the DNA vector has the presence of characteristic bands of linear and continuous DNA as compared to linear and non-continuous DNA when analyzed on a non-denaturing gel.

In some embodiments, the additional compound is encompassed in a second lipid nanoparticle. The first and the second lipid nanoparticles can be the same or different. In some embodiments, the first and second lipid nanoparticles are different. In some embodiments, the the first and second lipid nanoparticles are the same, i.e., the additional compound is encompassed in the first lipid nanoparticle.

Any desired molecule can be used as the additional compound. In some embodiments, the additional compound is a second capsid free, non-viral vector. The first and second capsid free, non-viral vectors can be the same or different. In some embodiments, the first and second capsid free, non-viral vectors are different.

In some embodiments, the additional compound is a therapeutic agent.

These and other aspects of the invention are described in further detail below.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A illustrates an exemplary structure of a ceDNA vector. In this embodiment, the exemplary ceDNA vector comprises an expression cassette containing CAG promoter, WPRE, and BGHpA. An open reading frame (ORF) encoding a Luciferase transgene is inserted into the cloning site (R3/R4) between the CAG promoter and WPRE. The expression cassette is flanked by two inverted terminal repeats (ITRs)—the wild-type AAV2 ITR on the upstream (5′-end) and a modified ITR on the downstream (3′-end) of the expression cassette, therefore the two ITRs flanking the expression cassette are asymmetric with respect to each other.

FIG. 1B illustrates an exemplary structure of a ceDNA vector with an expression cassette containing CAG promoter, WPRE, and BGHpA. An open reading frame (ORF) encoding Luciferase transgene is inserted into the cloning site between CAG promoter and WPRE. The expression cassette is flanked by two inverted terminal repeats (ITRs)—a modified ITR on the upstream (5′-end) and a wild-type ITR on the downstream (3′-end) of the expression cassette.

FIG. 1C illustrates an exemplary structure of a ceDNA vector with an expression cassette containing an enhancer/promoter, an open reading frame (ORF) for insertion of a transgene, a post transcriptional element (WPRE), and a polyA signal. An open reading frame (ORF) allows insertion of a transgene into the cloning site between CAG promoter and WPRE. The expression cassette is flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifiations).

FIG. 2A provides the T-shaped stem-loop structure of one wild-type ITR of AAV2 with identification of A-A′ arm, B-B′ arm, C-C′ arm, two Rep Binding sites (RBE and RBE') and the terminal resolution site (trs). The RBE contains a series of 4 duplex tetramers that are believed to interact with either Rep 78 or Rep 68. In addition, the RBE' is also believed to interact with Rep complex assembled on the wild-type ITR or mutated ITR in the construct. The D and D′ regions contain transcription factor binding sites and other conserved structure. FIG. 2B shows proposed Rep catalyzed nicking and ligating activities in the wild-type ITR of FIG. 2A, including the T-shaped stem-loop structure of the wild-type ITR of AAV2 with identification of A-A′ arm, B-B′ arm, C-C′ arm, two Rep Binding sites (RBE and RBE') and the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR (SEQ ID NO: 540). FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left) (SEQ ID NO: 113). FIG. 3C shows the primary structure (left) and the secondary structure (right) of the RBE-containing portion of the A-A′ loop, and the B-B′ and C-C′ arms of wild type right AAV2 ITR (SEQ ID NO: 541). FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right) (SEQ ID NO: 114). Any combination of left and right ITR (e.g., AAV2 ITRs or other viral serotype or synthetic ITRs) can be used, provided the left ITR is asymmetric or different from the right ITR. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of ceDNA in the process described in the schematic in FIG. 4B. FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as 1 kb and 2 kb in neutral conditions, but in a denaturing conditions, the stands remain connected and produce single strands that migrate as 2 kb and 4 kb. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions).

FIG. 5 is an exemplary picture of a denaturing gel running examples of ceDNA vectors with (+) or without (−) digestion with endonucleases (EcoRI for ceDNA construct 1 and 2; BamH1 for ceDNA construct 3 and 4; Spel for ceDNA construct 5 and 6; and XhoI for ceDNA construct 7 and 8). Sizes of bands highlighted with an asterisk were determined and provided on the bottom of the picture.

FIG. 6A is an exemplary Rep-bacmid in the pFBDLSR plasmid comprising the nucleic acid sequences for Rep proteins Rep52 and Rep78. This exemplary Rep-bacmid comprises: IE1 promoter fragment (SEQ ID NO:66); Rep78 nucleotide sequence, including Kozak sequence (SEQ ID NO:67), polyhedron promoter sequence for Rep52 (SEQ ID NO:68) and Rep58 nucleotide sequence, starting with Kozak sequence gccgccacc) (SEQ ID NO:69). FIG. 6B is a schematic of an exemplary ceDNA-plasmid-1, with the wt-L ITR, CAG promoter, luciferase transgene, WPRE and polyadenylation sequence, and mod-R ITR.

FIG. 7A shows results from an in vitro protein expression assay measuring Luciferase activity (y-axis, RQ (Luc)) in HEK293 cells 48 hours after transfection of 400 ng (black), 200 ng (gray), or 100 ng (white) of the constructs identified on the x-axis (construct-1, construct-3, construct-5, construct-7 (Table 5 in Example 1). FIG. 7B shows Luciferase activity (y-axis, RQ (Luc)) measured in HEK293 cells 48 hours after transfection of 400 ng (black), 200 ng (gray), or 100 ng (white) of the constructs identified on the x-axis (construct −2, construct −4, construct −6, construct −8) (Table 5). Luciferase activities measured in HEK293 cells treated with Fugene without any plasmids (“Fugene”), or in untreated HEK293 cells (“Untreated”) are also provided.

FIG. 8A shows viability of HEK293 cells (y-axis) 48 hours after transfection of 400 ng (black), 200 ng (gray), or 100 ng (white) of the constructs identified on the x-axis (construct-1, construct-3, construct-5, construct-7). FIG. 8B shows viability of HEK293 cells (y-axis) 48 hours after transfection of 400 ng (black), 200 ng (gray), or 100 ng (white) of the constructs identified on the x-axis (construct-2, construct-4, construct-6, construct-8).

FIGS. 9-11 are bar graphs showing average lipid nanoparticle size and ceDNA encapsulation of some exemplary lipid nanoparticles prepared with buffers comprising different salts at a constant N/P ratio (FIG. 9) or at varying N/P ratios (FIGS. 10 and 11).

FIG. 12 is a bar graph showing effect of serum/BSA on encapsulation in exemplary lipid nanoparticles.

FIG. 13 is a bar graph showing release of ceDNA from exemplary lipid nanoparticles in presence of dioleoylphosphatidylserine (DOPS) liposomes

FIG. 14 is a bar graph showing effect of serum/BSA on encapsulation in exemplary lipid nanoparticles.

FIG. 15 is a bar graph showing release of ceDNA from exemplary lipid nanoparticles in presence of dioleoylphosphatidylserine (DOPS) liposomes.

FIG. 16 shows ApoE binding of some exemplary lipid nanoparticles.

FIG. 16 is bar graph showing HEK293 expression of exemplary ceDNA.

FIG. 18 are gel electrophoresis photographs showing HEK293 expression of exemplary ceDNA.

FIG. 19 is a bar graph showing HEK293 expression of exemplary ceDNA.

FIG. 20 shows some exemplary compounds of Formula (I) and Formula (II) described in Example 10.

FIG. 21 is a generic synthetic scheme for synthesis of compounds of Formula (I) and (II) disclosed in Example 10.

FIG. 22 is a generic synthetic scheme for synthesis of compounds of Formula (I) and (II) disclosed in Example 10.

FIG. 23 shows some exemplary compounds of Formula (III) described in Example 11.

FIG. 24 depicts generic synthesis schemes for synthesis of compounds of Formula (III), Formula (IV) and Formula (V) described in Example 11.

### DETAILED DESCRIPTION

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “lipid nanoparticie” refers to a vesicle formed by one or more lipid componens Lipid nanoparticles are typically used as carriers for nucleic acid delivery in the context of pharmaceutical development. They work by fusing with a cellular membrane and repositioning its lipid structure to deliver a drug or active pharmaceutical ingredient (API). Generally, lipid nanoparticle compositions for such delivery are composed of synthetic ionizable or cationic lipids, phospholipids (especially compounds having a phosphatidylcholine group), cholesterol, and a polyethylene glycol (PEG) lipid; however, these compositions may also include other lipids. The sum composition of lipids typically dictates the surface characteristics and thus the protein (opsonization) content in biological systems thus driving biodistribution and cell uptake properties.

As used herein, the “liposome” refers to lipid molecules assembled in a spherical configuration encapsulating an interior aqueous volume that is segregated from an aqueous exterior. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “ionizable lipid” refers to lipids having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7. Ionizable lipids are also referred to as cationic lipids herein.

As used herein, the term “non-cationic lipid” refers to any amphipathic lipid as well as any other neutral lipid or anionic lipid. Accordingly, the non-cationic lipid can be a neutral uncharged, zwitterionic, or anionic lipid.

As used herein, the term “conjugated lipid” refers to a lipid molecule conjugated with a non-lipid molecule, such as a PEG, polyoxazoline, polyamide, or polymer (e g., cationic polymer).

As used herein, the term “excipient” refers to pharmacologically inactive ingredients that are included in a formulation with the API, e.g., ceDNA and/or lipid nanoparticles to bulk up and/or stabilize the formulation when producing a dosage form. General categories of excipients include, for example, bulking agents, fillers, diluents, antiadherents, binders, coatings, disintegrants, flavours, colors, lubricants, glidants, sorbents, preservatives, sweeteners, and products used for facilitating drug absorption or solubility or for other pharmacokinetic considerations.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein. Transgenes of interest include, but are not limited to, nucleic acids encoding polypeptides, preferably therapeutic (e.g., for medical, diagnostic, or veterinary uses) or immunogenic polypeptides (e.g., for vaccines). In some embodiments, nucleic acids of interest include nucleic acids that are transcribed into therapeutic RNA. Transgenes included for use in the ceDNA vectors of the invention include, but are not limited to, those that express or encode one or more polypeptides, peptides, ribozymes, aptamers, peptide nucleic acids, siRNAs, RNAis, miRNAs, lncRNAs, antisense oligo- or polynucleotides, antibodies, antigen binding fragments, or any combination thereof.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species.

As used herein, the term “asymmetric ITRs” refers to a pair of ITRs within a single ceDNA genome or ceDNA vector that are not inverse complements across their full length. The difference in sequence between the two ITRs may be due to nucleotide addition, deletion, truncation, or point mutation. In one embodiment, one ITR of the pair may be a wild-type AAV sequence and the other a non-wild-type or synthetic sequence. In another embodiment, neither ITR of the pair is a wild-type AAV sequence and the two ITRs differ in sequence from one another. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis—acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site”, Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEC) ID NO: 531), an RBS sequence identified in AA V2 Any known RBS sequence may be used in the embodiments of the invention, including other known AAV RBS sequences and other naturally known or synthetic RB S sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 531). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 45), the hexanucleotide sequence identified in AAV2. Any known TRS sequence may be used in the embodiments of the invention, including other known AAV TRS sequences and other naturally known or synthetic TRS sequences such as AGTT (SEQ ID NO: 46), GGTTGG (SEQ ID NO: 47), AGTTGG (SEQ ID NO: 48), AGTTGA (SEQ ID NO: 49), and other motifs such as RRTTRR (SEQ ID NO: 50).

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the terms “closed-ended DNA vector”, “ceDNA vector” and “ceDNA” are used interchangeably and refer to a non-virus capsid-free DNA vector with at least one covalently-closed end (i.e., an intramolecular duplex). In some embodiments, the ceDNA comprises two covalently-closed ends.

As defined herein, “reporters” refer to proteins that can be used to provide deteactable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself, or that of another promoter used in another modular component of the synthetic biological circuits described herein. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herien.

The term “enhancer” as used herein refers a cis-acting regulatory sequence (e.g., 50-1,500 base pairs) that bind one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, for example, U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The term “subject” as used herein refers to a human or animal, to whom treatment, including prophylactic treatment, with the ceDNA vector according to the present invention, is provided. Usually the animal is a vertebrate such as, but not limited to a primate, rodent, domestic animal or game animal. Primates include but are not limited to chimpanzees, cynomologous monkeys, spider monkeys, and macaques, e.g., Rhesus. Rodents include mice, rats, woodchucks, ferrets, rabbits and hamsters. Domestic and game animals include but are not limited to cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment.

As used herein, the term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the same antigen to which the intact antibody binds. In one embodiment, the antibody or antibody fragment comprises an immunoglobulin chain or fragment thereof and at least one immunoglobulin variable domain sequence. Examples of antibodies and antibody fragments include, but are not limited to, an Fv, an scFv, a Fab fragment, a Fab′, a F(ab′)₂, a Fab′-SH, a single domain antibody (dAb), a heavy chain, a light chain, a heavy and light chain, a full antibody (e.g., includes each of the Fc, Fab, heavy chains, light chains, variable regions etc.), a bispecific antibody, a diabody, a linear antibody, a single chain antibody, an intrabody, a monoclonal antibody, a chimeric antibody, a multispecific antibody, or a multimeric antibody. An antibody or antibody fragment can be of any class, including but not limited to IgA, IgD, IgE, IgG, and IgM, and of any subclass thereof including but not limited to IgG1, IgG2, IgG3, IgG4, IgA1 and IgA2. In addition, an antibody can be derived from any mammal, for example, primates, humans, rats, mice, horses, goats etc. In one embodiment, the antibody is human or humanized. In some embodiments, the antibody is a modified antibody. In some embodiments, the components of an antibody can be expressed separately such that the antibody self-assembles following expression of the protein components. In some embodiments, the antibody has a desired function, for example, interaction and inhibition of a desired protein for the purpose of treating a disease or a symptom of a disease. In one embodiment, the antibody or antibody fragment comprises a framework region or an F, region.

As used herein, the term “antigen-binding domain” of an antibody molecule refers to the part of an antibody molecule, e.g., an immunoglobulin (Ig) molecule, that participates in antigen binding. In embodiments, the antigen binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions,” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4.

As used herein, the term “full length antibody” refers to an immunoglobulin (Ig) molecule (e.g., an IgG antibody), for example, that is naturally occurring, and formed by normal immunoglobulin gene fragment recombinatorial processes.

As used herein, the term “functional antibody fragment” refers to a fragment that binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean ±1%. The present invention is further explained in detail by the following examples, but the scope of the invention should not be limited thereto.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Without limitations, a lipid nanoparticle of the invention includes a lipid formulation that can be used to deliver a capsid-free, non-viral DNA vector to a target site of interest (e.g., cell, tissue, organ, and the like). Generally, the lipid nanoparticle comprises capsid-free, non-viral DNA vector and an ionizable lipid or a salt thereof.

Accordingly, in some aspects, the disclosure provides for a lipid nanoparticle comprising ceDNA and an ionizable lipid. For example, a lipid nanoparticle formulation that is made and loaded with ceDNA obtained by the process of Example 1 or otherwise disclosed herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in the PCT and US patent publications listed in Table 1, thecontents of all of which are incorporated herein by reference in their entirety.

TABLE 1
Ionizable lipids

| PCT Publication | U.S. Publication |
|---|---|
| WO2015/095340 | U.S. 2016/0311759 |
| WO2015/199952 | U.S. 2015/0376115 |
| WO2018/011633 | U.S. 2016/0151284 |
| WO2017/049245 | U.S. 2017/0210697 |
| WO2015/061467 | U.S. 2015/0140070 |
| WO2012/040184 | U.S. 2013/0178541 |
| WO2012/000104 | U.S. 2013/0303587 |
| WO2015/074085 | U.S. 2015/0141678 |
| WO2016/081029 | U.S. 2015/0239926 |
| WO2017/004143 | U.S. 2016/0376224 |
| WO2017/075531 | U.S. 2017/0119904 |
| WO2017/117528 | |
| WO2011/022460 | U.S. 2012/0149894 |
| WO2013/148541 | U.S. 2015/0057373 |
| WO2013/116126 | |
| WO2011/153120 | U.S. 2013/0090372 |
| WO2012/044638 | U.S. 2013/0274523 |
| WO2012/054365 | U.S. 2013/0274504 |
| WO2011/090965 | U.S. 2013/0274504 |
| WO2013/016058 | |
| WO2012/162210 | |
| WO2008/042973 | U.S. 2009/0023673 |
| WO2010/129709 | U.S. 2012/0128760 |
| WO2010/144740 | U.S. 201/003241240 |
| WO2012/099755 | U.S. 2014/0200257 |
| WO2013/049328 | U.S. 2015/0203446 |
| WO2013/086322 | U.S. 2018/0005363 |
| WO2013/086373 | U.S. 2014/0308304 |
| WO2011/071860 | U.S. 2013/0338210 |
| WO2009/132131 | |
| WO2010/048536 | |
| WO2010/088537 | U.S. 2012/0101148 |
| WO2010/054401 | U.S. 2012/0027796 |
| WO2010/054406 | |
| WO2010/054405 | |
| WO2010/054384 | U.S. 2012/0058144 |
| WO2012/016184 | U.S. 2013/0323269 |
| WO2009/086558 | U.S. 2011/0117125 |
| WO2010/042877 | U.S. 2011/0256175 |
| WO2011/000106 | U.S. 2012/0202871 |
| WO2011/000107 | U.S. 2011/0076335 |
| WO2005/120152 | U.S. 2006/0083780 |
| WO2011/141705 | U.S. 2013/0123338 |
| WO2013/126803 | U.S. 2015/0064242 |
| WO2006/07712 | U.S. 2006/0051405 |
| WO2011/038160 | U.S. 2013/0065939 |
| WO2005/121348 | U.S. 2006/0008910 |
| WO2011/066651 | U.S. 2003/0022649 |
| WO2009/127060 | U.S. 2010/0130588 |
| WO2011/141704 | U.S. 2013/0116307 |
| WO2006/069782 | U.S. 2010/0062967 |
| WO2012/031043 | U.S. 2013/0202684 |
| WO2013/006825 | U.S. 2014/0141070 |
| WO2013/033563 | U.S. 2014/0255472 |
| WO2013/089151 | U.S. 2014/0039032 |
| WO2017/099823 | U.S. 2018/0028664 |
| WO2015/095346 | U.S. 2016/0317458 |
| WO2013/086354 | U.S. 2013/0195920 |

In some embodiments, the ionizable lipid is a compound of Formula (X),

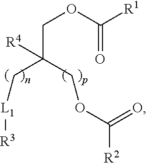

as defined in US2016/0311759, the content of which is incorporated herein by reference in its entirety.

In some embodiments, the ionizable lipid is a compound of Formula (I),

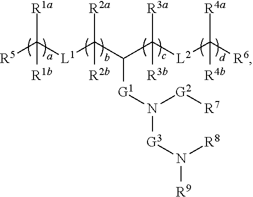

as defined in US20150376115 or in US2016/0376224, the content of both of which is incorporated herein by reference in its entirety.

In some embodiments, the ionizable lipid is a compound of Formula (I),

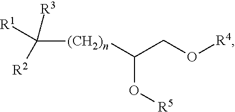

### or Formula (II),

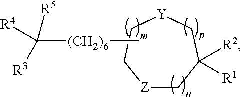

### or Formula (III),

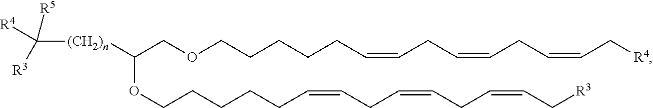

as defined in US20160151284, the content of which is incorporated herein by reference in its entirety.

In some embodiments, the ionizable lipid is a compound of Formula (I),

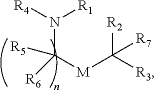

### or Formula (IA),

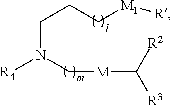

### or Formula (II),

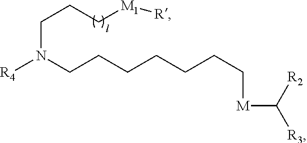

### or Formula (IIA),

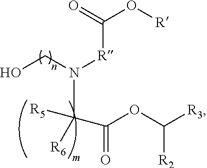

as defined in US20170210967, the content of which is incorporated herein by reference in its entirety.

In some embodiments, the ionizable lipid is compound of Formula I-c,

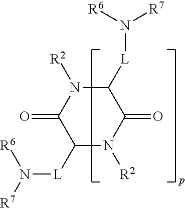

as defined in US20150140070, the content of which is incorporated herein by reference in its entirety.

In some embodiments, the ionizable lipid is a compound of Formula A,

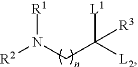

as defined in US2013/0178541, the content of which is incorporated herein by reference in its entirety.

In some embodiments, the ionizable lipid is a compound of Formula (I),

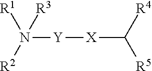

as defined in US2013/0303587 or in US2013/0123338, the content of both which is incorporated herein by reference in its entirety.

In some embodiments, the ionizable lipid is a compound of Formula (I),

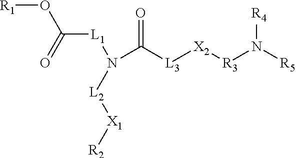

as defined in US2015/0141678, the content of which is incorporated herein by reference in its entirety.

In some embodiments, the ionizable lipid is a compound of Formula (II),

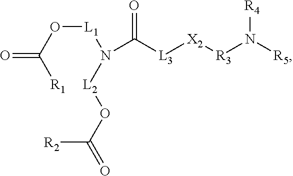

### Formula (III),

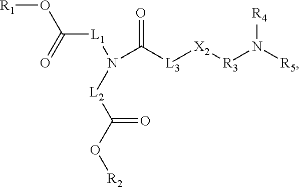

### Formula (IV),

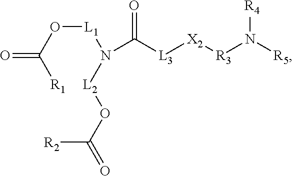

### or Formula (V),

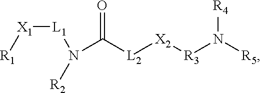

as defined in US2015/0239926, the content of which is incorporated herein by reference in its entirety.

In some embodiments, the ionizable lipid is a compound of Formula (I),

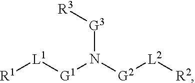

as defined in US2017/0119904, the content of which is incorporated herein by reference in its entirety.

In some embodiments, the ionizable lipid is a compound of Formula (I) or Formula (II), each having the structure:

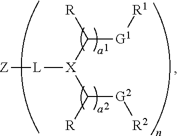

as defined in WO2017/117528, the content of which is incorporated herein by reference in its entirety.

In some embodiments, the ionizable lipid is a compound of Formula A,

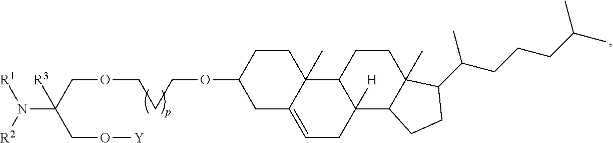

as defined in US2012/0149894, the content of which is incorporated herein by reference in its entirety.

In some embodiments, the ionizable lipid is a compound of Formula A,

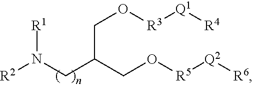

as defined in US2015/0057373, the content of which is incorporated herein by reference in its entirety.

In some embodiments, the ionizable lipid is a compound of Formula A,

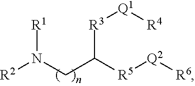

as defined in WO2013/116126, the content of which is incorporated herein by reference in its entirety.

In some embodiments, the ionizable lipid is a compound of Formula A,

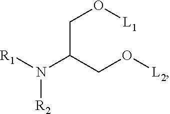

as defined in US2013/0090372, the content of which is incorporated herein by reference in its entirety.

In some embodiments, the ionizable lipid is a compound of Formula A,

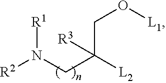

as defined in US2013/0274523, the content of which is incorporated herein by reference in its entirety.

In some embodiments, the ionizable lipid is compound of Formula A,

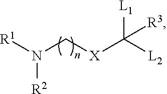

as defined in US2013/0274504, the content of which is incorporated herein by reference in its entirety.

In some embodiments, the ionizable lipid is a compound of Formula A,

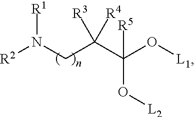

as defined in US2013/0053572, the content of which is incorporated herein by reference in its entirety.

In some embodiments, the ionizable lipid is a compound of Formula A,

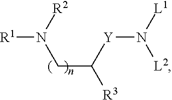

as defined in WO2013/016058, the content of which is incorporated herein by reference in its entirety.

In some embodiments, the ionizable lipid is a compound of Formula A,

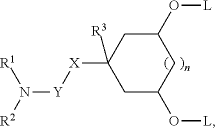

as defined in WO2012/162210, the content of which is incorporated herein by reference in its entirety.

In some embodiments, the ionizable lipid is a compound of Formula (I),

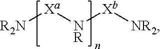

as defined in US2008/042973, the content of which is incorporated herein by reference in its entirety.

In some embodiments, the ionizable lipid is a compound of Formula (I),

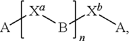

### Formula (II),

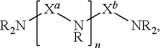

### Formula (III),

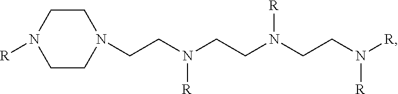

### or Formula (IV),

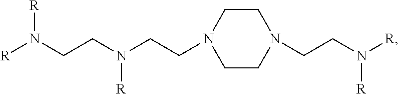

as defined in US2012/01287670, the content of which is incorporated herein by reference in its entirety.

In some embodiments, the ionizable lipid is a compound of Formula (I) or Formula (II), each having the structure:

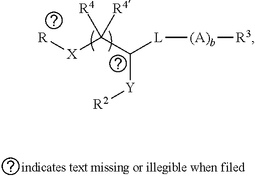

as defined in US2014/0200257, the content of which is incorporated herein by reference in its entirety.

In some embodiments, the ionizable lipid is a compound of Formula (I), Formula (II) of Formula (III), each having the structure:

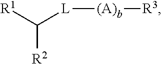

as defined in US2015/0203446, the content of which is incorporated herein by reference in its entirety.

In some embodiments, the ionizable lipid is a compound of Formula (I),

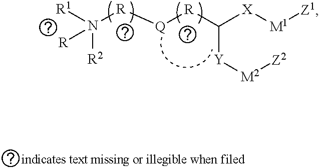

### or Formula (III),

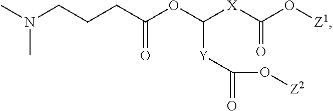

as defined in US2015/0005363, the content of which is incorporated herein by reference in its entirety.

In some embodiments, the ionizable lipid is a compound of Formula (I),

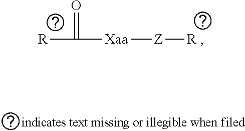

### Formula (IA),

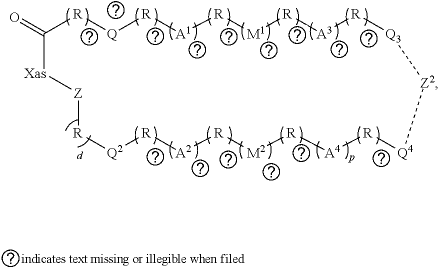

### Formula (IB),

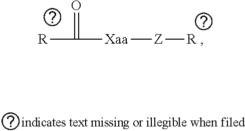

### Formula (IC),

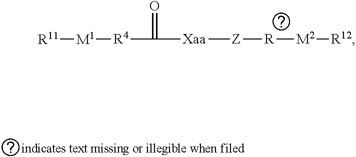

### Formula (ID,

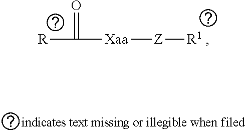

### Formula (II),

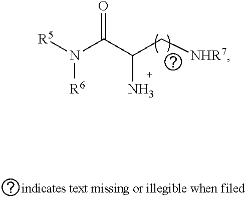

### Formula (IIA),

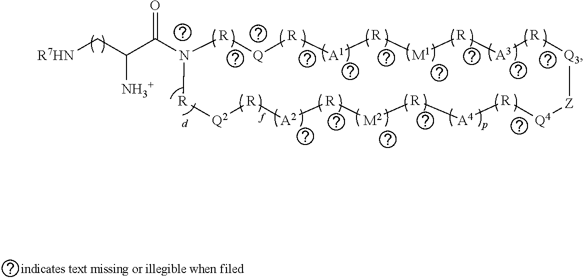

### Formula (IIB),

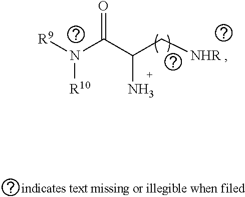

### Formula (IIC),

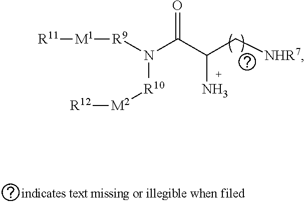

Formula (IID), or Formula (III)-(XXIV), as defined in US2014/0308304, the content of which is incorporated herein by reference in its entirety.

In some embodiments, the ionizable lipid is a compound of formula

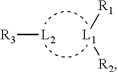

as defined in US2013/0338210, the content of which is incorporated herein by reference in its entirety.

In some embodiments, the ionizable lipid is a compound of Formula ((I),

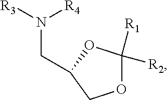

### Formula (II),

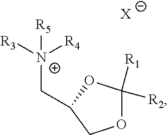

### Formula (III)

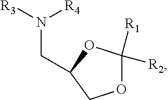

### or Formula (IV),

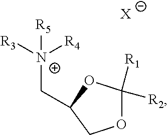

as defined in WO2009/132131, the content of which is incorporated herein by reference in its entirety.

In some embodiments, the ionizable lipid is a compound of Formula A,

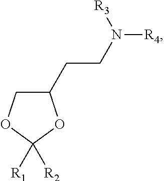

as defined in US2012/01011478, the content of which is incorporated herein by reference in its entirety.

In some embodiments, the ionizable lipid is a compound of Formula (I),

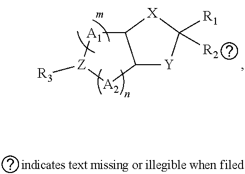

### or Formula (XXXV),

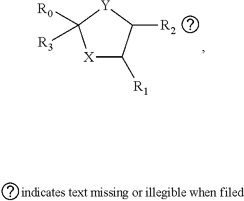

as defined in US2012/0027796, the content of which is incorporated herein by reference in its entirety.

In some embodiments, the ionizable lipid is a compound of Formula (XIV),

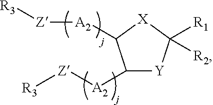

### or Formula (XVII),

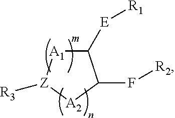

as defined in US2012/0058144, the content of which is incorporated herein by reference in its entirety.

In some embodiments, the ionizable lipid is a compound of formula

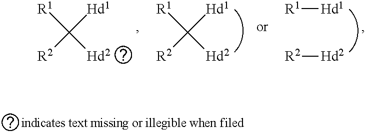

as defined in US2013/0323269, the content of which is incorporated herein by reference in its entirety.

In some embodiments, the ionizable lipid is a compound of Formula (I),

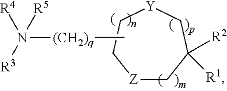

as defined US2011/0117125, the content of which is incorporated herein by reference in its entirety.

In some embodiments, the ionizable lipid is a compound of Formula (I),

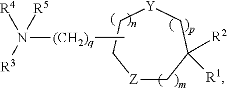

### Formula (II),

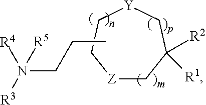

### or Formula (III),

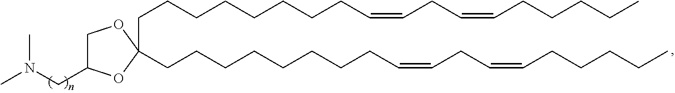

as definedinUS2011/0256175, the content of which is incorporated herein by reference in its entirety.

In some embodiments, the ionizable lipid is a compound of Formula (I),

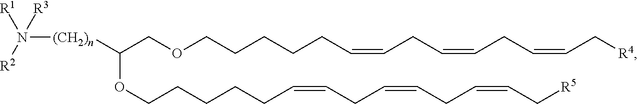

### Formula (II),

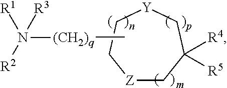

### Formula (III),

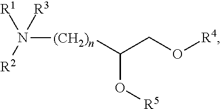

### Formula (IV),

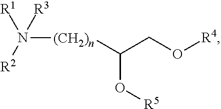

### Formula (V),

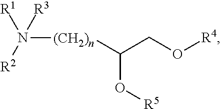

### Formula (VI),

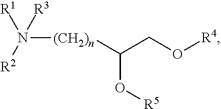

### Formual (VII),

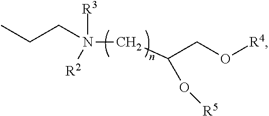

### Formula (VIII),

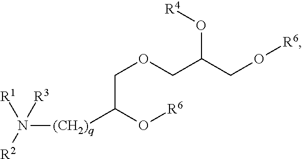

### Formula (IX),

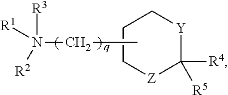

### Formula (X),

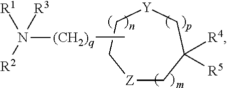

### -Formula (XI),

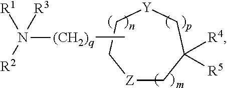

### or Formula (XII),

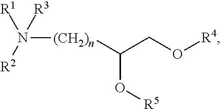

as defined in US2012/0202871, the content of which is incorporated herein by reference in its entirety.

In some embodiments, the ionizable lipid is compound of Formula (I),

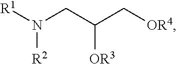

### Formula (II),

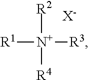

### Formula (III),

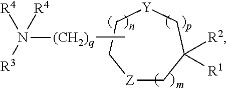

### Formula (IV),

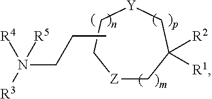

### Formula (V),

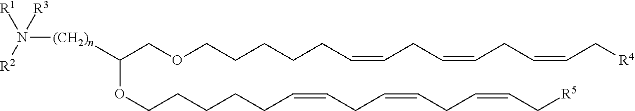

### Formula (VI),

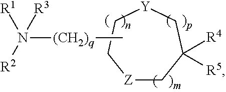

### Formula (VII),

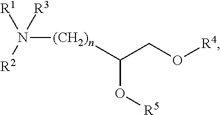

### Formula (VIII),

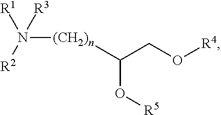

### Formula (IX),

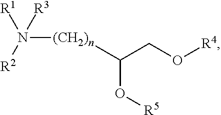

### Formula (X),

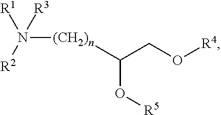

### Formula (XI),

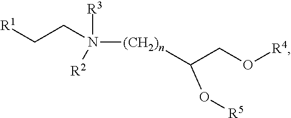

### Formula (XII),

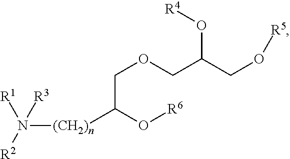

### Formula (XIII),

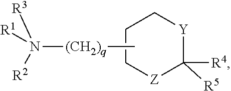

### Formula (XIV),

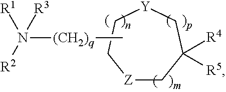

### Formula (XV),

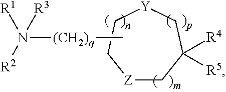

### or Formula (XVI),

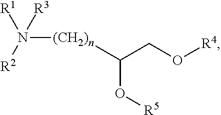

as defined in US2011/0076335, the content of which is incorporated herein by reference in its entirety.

In some embodiments, the ionizable lipid is a compound of Formula (I),

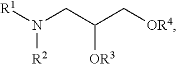

### or Formula (II),

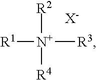

as defined in US2006/008378, the content of which is incorporated herein by reference in its entirety.

In some embodiments, the ionizable lipid is a compound of Formula (I),

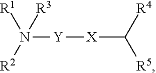

as defined in US2013/0123338, the content of which is incorporated herein by reference in its entirety.

In some embodiments, the ionizable lipid is a compound of Formula (I), X-A-Y-Z, as defined in US2015/0064242, the content of which is incorporated herein by reference in its entirety.

In some embodiments, the ionizable lipid is a compound of Formula (XVIX),

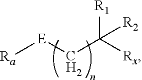

### Formula (XVII),

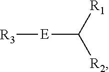

### or Formula (XVIII),

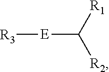

as defined in US2013/0022649, the content of which is incorporated herein by reference in its entirety.

In some embodiments, the ionizable lipid is compound of Formula (I),

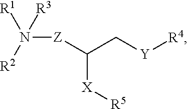

### Formula (II),

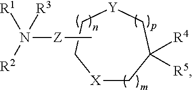

### or Formula (III),

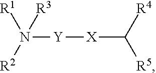

as defined in US2013/0116307, the content of which is incorporated herein by reference in its entirety.

In some embodiments, the ionizable lipid is compound of Formula (I),

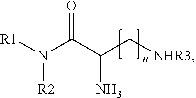

### or Formula (II),

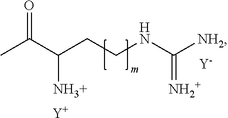

as defined in US2010/0062967, the content of which is incorporated herein by reference in its entirety.

In some embodiments, the ionizable lipid is a compound of Formula (I)-(X), each having the structure:

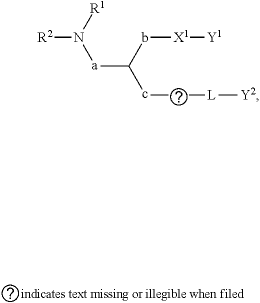

as defined in US2013/0189351, the content of which is incorporated herein by reference in its entirety.

In some embodiments, the ionizable lipid is a compound of Formula (I),

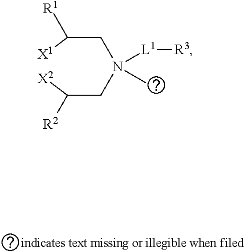

as defined in US2014/0039032, the content of which is incorporated herein by reference in its entirety.

In some embodiments, the ionizable lipid is a compound of Formula (V),

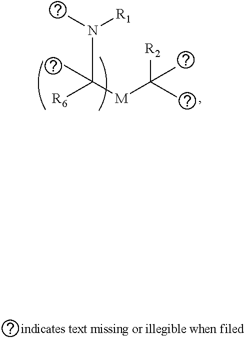

as defined in US2018/0028664, the content of which is incorporated herein by reference in its entirety.

In some embodiments, the ionizable lipid is a compound of Formula (I),

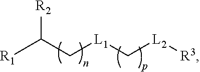

as defined in US2016/0317458, the content of which is incorporated herein by reference in its entirety.

In some embodiments, the ionizable lipid is a compound of Formula (I),

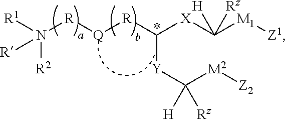

as defined in US2013/0195920, the content of which is incorporated herein by reference in its entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) described in Example 9.

In some embodiments, the ionizable lipid is the lipid ATX-002 described in Example 10.

In some embodiments, the ionizable lipid is (13Z,16Z)-N,N-dimethyl-3-nonyldocosa-13,16-dien-1-amine (Compound 32) described in Example 11.

In some embodiments, the ionizable lipid is Compound 6 or Compound 22 described in Example 12.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids include, but are not limited to, distearoyl-sn-glycero-phosphoethanolamine, di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), di stearoyl-phosphatidyl-ethanolamine (DSPE), monomethyl-phosphatidylethanolamine (such as 16-O-monomethyl PE), dimethyl-phosphatidylethanolamine (such as 16-0-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), di stearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Other examples of non-cationic lipids suitable for use in the lipid nanoparticles include nonphosphorous lipids such as, e.g., stearylamine, cltpdecylamine, hexaclecylarnine, acetyl palmitate, glycerolricinoleate, hexadecyl stereate, isopropyl myri state, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid is a phospholipid. In some embodiments, the non-cationic lipid is selected from DSPC, DPPC, DMPC, DOPC, POPC, DOPE, and SM. In some preferred embodiments, the non-cationic lipid is DPSC.

Exemplary non-cationic lipids are described in PCT Publication WO2017/099823 and US patent publication US2018/0028664, the contents of both of which are incorporated herein by reference in their entirety.

In some examples, the non-cationic lipid is oleic acid or a compound of Formula (I),

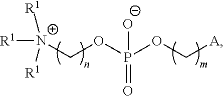

### Formula (II)

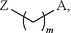

### or Formula (IV),

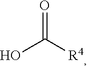

as defined in US2018/0028664, the content of which is incorporated herein by reference in its entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids.

In some aspects, the lipid nanoparticle can further comprise a component, such as a sterol, to provide membrane integrity.

One exemplary sterol that can be used in the lipid nanoparticle is cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, Six-cholestanone, 5β-cholestanone, and cholesteryl decanoate; and mixtures thereof. In some embodiments, the cholesterol derivative is a polar analogue such as cholesteryl-(4′-hydroxy)-butyl ether.

Exemplary cholesterol derivatives are described in PCT publication WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety.

In some embodiments, a PEG-lipid is a compound of Formula (III),

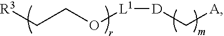

### Formula (III-a-I),

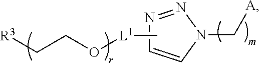

### Formula (III-a-2),

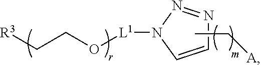

### Formula (III-b-1),

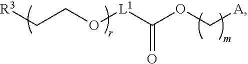

### Formula (III-b-2),

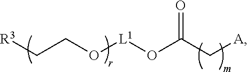

### or Formula (V),

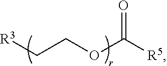

as defined in US2018/0028664, the content of which is incorporated herein by reference in its entirety.

In some embodiments, a PEG-lipid is of Formula (II),

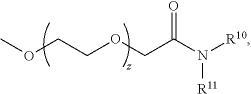

as defined in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-di stearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3 -phosphoethanol amine-N-[methoxy(polyethylene glycol)-2000],

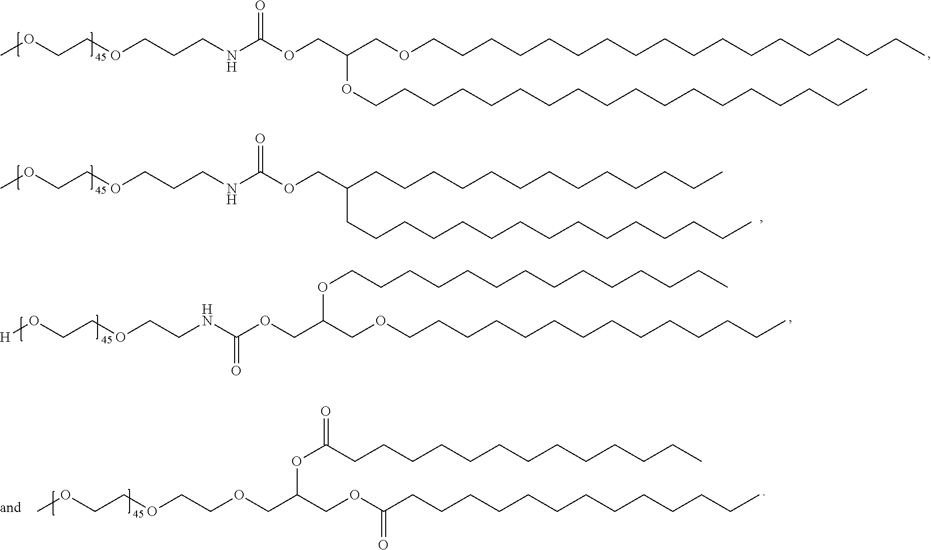

Lipids conjugated with a molecule other than a PEG can also be used in place of PEG-lipid. For example, polyoxazoline (PCZ)-lipid conjugates, polyamide-lipid conjugates (such as ATTA-lipid conjugates), and cationic-polymer lipid (CPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and US patent applications listed in Table 2, the contents of all of which are incorporated herein by reference in their entirety.

TABLE 2
Conjugated lipids

| PCT Publication | US Publication |
|---|---|
| WO1996/010392 | U.S. Pat. No. 5,885,613 |
| WO1998/051278 | U.S. Pat. No. 6,287,591 |
| WO2002/087541 | U.S. 2003/0077829 |
| WO2005/026372 | U.S. 2005/0175682 |
| WO2008/147438 | U.S. 2008/0020058 |
| WO2009/086558 | U.S. 2011/0117125 |
| WO2012/000104 | U.S. 2013/0303587 |
| WO2017/117528 | |
| WO2017/099823 | U.S. 2018/0028664 |
| WO2015/199952 | U.S. 2015/0376115 |
| WO2017/004143 | U.S. 2016/0376224 |
| WO2015/095346 | U.S. 2016/0317458 |
| | U.S. Pat. No. 6,320,017 |
| | U.S. Pat. No. 6,586,559 |
| WO2012/000104 | U.S. 2013/0303587 |
| WO2012/000104 | U.S. 2013/0303587 |
| WO2010/006282 | U.S. 20110123453 |

The PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle.

Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In other aspects, the disclosure provides for a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the ceDNA or at least a second ceDNA, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the ceDNA is useful for treating an infection, the additional compound can be an antimicrobial agent (e.g., an antibiotic or antiviral compound). In yet another example, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a ceDNA encoding a different protein or a different compound, such as a therapeutic may be used in the compositions and methods of the invention.

In some embodiments, the additional compound is an immune modulating agent. For example, the additional compound is an immunosuppressant. In some embodiments, the additional compound is immunostimulatory.

Exemplary immune modulators include, but are not limited to, interleukins (e.g., IL-2, IL-7, IL-12), cytokines (e.g., granulocyte colony-stimulating factor (G-CSF), interferons), chemokines (e.g., CCL3, CCL26, CXCL7), Immunomodulatory imide drugs (IMiDs) (e.g., thalidomide and its analogues (lenalidomide, pomalidomide, and apremilast), other immune modulators, including but not limited to: cytosine phosphate-guanosine, oligodeoxynucleotides, glucans.

In some embodiments, the immune modulator can be an immunosuppresive drug. Exemplary immunosuppressive drugs include, but not limited to, glucocorticoids, cytostatics, antibodies, drugs acting on immunophilins and other drugs. Glucocorticoids include but are not limited to, prednisone, dexamethasone, and hydrocortisone. Examples of cytostatics include alkylating agents such as nitrogen mustards (e.g., cyclophosphamide), nitrosoureas, and platinum compounds. Cytostatics can also include the antimetabolites such as folic acid analogues (e.g., methotrexate), purine analogues (e.g., azathioprine and mercaptopurine), pyrimidine analogues (e.g., fluorouracil) and protein synthesis inhibitors. Other cystostatics include cytotoxic antibiotics such as dactinomycins, anthracyclines, mitomycin C, bleomycin, and mithramycin.

Antibodies for immune suppression include, but are not limited to, Atgam, obtained from horse serum, and Thymoglobuline, antibodies directed to the IL-2 receptor-(CD25-) and/or CD3-directed antibodies, MUROMONAB-CD3™ (Orthoclone OKT3), basiliximab (SIMULECT™), daclizumab (ZENAPAX™) and muromonab.

Drugs acting on immunphillins include, but are not limited to, ciclosporin, tacrolimus, rapamycin (SIROLIMUS™) and Everolimus. Examples of biologics include abatacept, anakinra, certolizumab, golimumab, ixekizumab, natalizumab, rituximab, secukinumab, tocilizumab, ustekinumab and vedolizumab.

Other drugs useful as immune modulators or immune suppressors include, but are not limited to, interferons (e.g., IFN-β), opioids, TNF binding proteins (e.g., TNF-α (tumor necrosis factor-alpha) binding protein, infliximab (REMICADE™), etanercept (ENBREL™) or adalimumab (HUMIRA™)), curcumin (an ingredient in turmeric) and catechins (in green tea), Mycophenolate, Fingolimod, myriocin, antiproliferative agents (e.g., myriocin, tacrolimus, mycophenolate mofetil, mycophenolate sodium, azathioprine), mTOR inhibitors (e.g., sirolimus and everolimus), calcineurin inhibitors (e.g., cyclosporine and tacrolimus), IMDS inhibitors (e.g., azathioprine, leflunomide, and mycophenolate), fingolmod, abatacept, anakinra, certolizumab, golimumab, ixekizumab, natalizumab, rituximab, secukinumab, tocilizumab, ustekinumab and vedolizumab.

In some embodiments, immunosuppressive agents useful in the compositions and methods as disclosed herein can be selected from one of the following compounds: mycophenolic acid, cyclosporin, azathioprine, tacrolimus, cyclosporin A, FK506, rapamycin, leflunomide, deoxyspergualin, prednisone, azathioprine, mycophenolate mofetil, OKT3, ATAG or mizoribine.

In certain embodiments, an immune suppressants is selected from the group consisting of Prednisone, methylprednisolone, Kenalog, Medrol Oral, Medrol (Pak) Oral, Depo-Medrol Inj, prednisolone Oral, Solu-Medrol Inj, hydrocortisone Oral, Cortef Oral, Solu-Medrol IV, cortisone Oral, Celestone Soluspan Inj, Orapred ODT Oral, Orapred Oral, Prelone Oral, methylprednisolone acetate Inj , Prednisone Intensol Oral, betamethasone acet & sod phos Inj, Veripred, Celestone Oral, methylprednisolone sodium succ IV, methylprednisolone sodium succ Inj, Millipred Oral, Solu-Medrol (PF) Inj, Solu-Cortef Inj, Aristospan Intra-Articular Inj, hydrocortisone sod succinate Inj, prednisolone sodium phosphate Oral, methylprednisolone sod suc(PF) IV, Solu-Medrol (PF) IV, triamcinolone hexacetonide Inj, A-Hydrocort Inj, A-Methapred Inj, Millipred DP Oral, Flo-Pred Oral, Aristospan Intralesional Inj, betamethasone Oral, methylprednisolone sod suc(PF) Inj, hydrocortisone sod succ (PF) Inj, Solu-Cortef (PF) Inj, prednisolone acetate Oral, dexamethasone in 0.9% NaCl IV, Rayos, levothyroxine. Of course, immune suppressants are which are known to those of ordinary skill in the art can readily be substituted as this list should not be considered exhaustive or limiting.

An immune suppressant can result in the reduction of immune cells in the subject, e.g., a reduction of immune cells which express at least one or more of: CD11b, CD4, CD8, and/or a reduction in pro-inflammatory cytokines selected from, but not limited to, TNFa or MCP-1. In some embodiments, a pro-inflammatory cytokine is selected from any one or a combination of: cytokines, lymphokines, monokines, stem cell growth factors, lymphotoxins, hematopoietic factors, colony stimulating factors (CSF), interferons (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, transforming growth factor (TGF), TGF-α, TGF-β., insulin-like growth factor (IGF), erythropoietin, thrombopoietin, tumor necrosis factor (TNF), TNF-α., TNF-β, mullerian-inhibiting substance (MIS), mouse gonadotropin-associated peptide, inhibin, activin, vascular endothelial growth factor, integrin, interleukin (IL), granulocyte-colony stimulating factor (G-CSF), granulocyte macrophage-colony stimulating factor (GM-CSF), interferon-.alpha., interferon-.beta., interferon-.gamma., S1 factor, IL-1, IL-1 cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21, IL-23, IL-25, LIF, kit-ligand, FLT-3, angiostatin, thrombospondin and endostatin.

In some embodiments, a pro-inflammatory cytokine can be selected from any or a combination of interleukin-1β (IL-1β), tumor necrosis factor-α (TNF-α), interleukin-6 (IL-6), interleukin-8 (IL-8), interferon-γ (IFN-γ), vascular endothelial growth factor (VEGF), leukemia inhibitory factor (LIF), monocyte chemoattractant protein-1 (MCP-1), RANTES, interleukin-10 (IL-10), interleukin-12 (IL-12), matrix metalloproteinase 2 (MMP2), IP-10, macrophage inflammatory protein 1α (MIP1a) and/or macrophage inflammatory protein 1β (MIP 1(3).

In some embodiments, the immune modulating agent is a NLRP3 agonist. Exemplary NLRP3 agonists include but are not limited to an imadazoquinoline; an imidazonaphthyridine; a pyrazolopyridine; an aryl-substituted imidazoquinoline; a compound having a 1-alkoxy 1H-imidazo ring system; an oxazolo [4,5-c]-quinolin-4-amine; an thiazolo [4,5-c]-quinolin-4-amine; a selenazolo [4,5-c]-quinolin-4-amine; an imidazonaphthyridine, an imidazoquinolinamine; a 1-substituted, 2-substituted 1H-imidazo[4,5-C]quinolin-4-amine; a fused cycloalkylimidazopyridine; a 1H-imidazo[4,5-c]quinolin-4-amine; a 1-substituted 1H-imidazo-[4,5-c]quinolin-4-amine; an imidazo-[4,5-C]quinolin-4-amine; a 2-ethyl 1H-imidazo[4,5-ciquinolin-4-amine; an olfenic 1H-imidazo[4,5-c]quinolin-4-amine; a 6,7-dihydro-8-(imidazol-1 -yl)-5 -methyl-1 -oxo-1H,5H-benzo[ij ]quinolizine-2-carboxylic acid; a pyridoquinoxaline-6-carboxylic acid; a 6,7-dihydro-8-(imidazol-1-yl)-5-methyl-1-oxo-1H,5H-benzo [ij]quinolizine-2-carboxylic acid; a substituted naphtho[ij]quinolizine; a substituted pyridoquinoxaline-6-carboxylic acid; a 7-hydroxy-benzo[ij]quinolizine-2-carboxylic acid derivative; a substituted benzo[ij]quinolizine-2-carboxylic acid; a7-hydroxy-benzo[ij]quinolizine-2-carboxylic acid; a substituted pyrido[1,2,3,-de]-1,4-benzoxazine; and a N-methylene malonate of tetrahydroquinoline. In some embodiments, the NLRP3 agonist is a compound of formula:

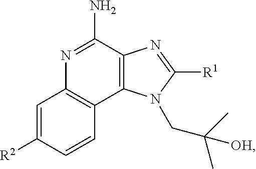

as defined in US20170056448A1, content of which is incorporated herein by reference in its entirety.

In some embodiments, the immune modulating agent is a TLR7 and/or TLR8 ligand. In some embodiments, the immune modulating agent is imiquimod (1-isobutyl-1H-imidazo[4,5-c]quinolin-4-amine) or resiquimod.

In some embodiments, the immune modulating agent is a Imquidazolequinoline-based compound of formula:

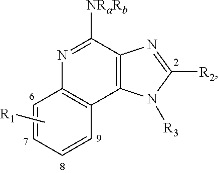

as defined in US9034336B2, content of which is incorporated herein by reference in its entirety.

In some embodiments, the immune modulating agent is a SMAD7 modulator. For example, the SMAD7 modulator can be SMAD7 antisense oligonucleotide (AON) as defined in WO2017059225A1, content of which is incorporated herein by reference in its entirety.

The immune modulating agent can be a TLR modulator. For example, the immune modulating agent can be a TLR3, TLR4, TLR7, TLR8, or TLR9 modulator, such as a TLR3, TLR4, TLR7, TLR8, or TLR9 agonist or a TLR3, TLR4, TLR7, TLR8, or TLR9 antagonist. In some embodiments, the TLR modulator is a TLR3 modulator. In some embodiments, the TLR modulator is a TLR4 modulator. In some embodiments, the TLR modulator is a TLR7 modulator. In some embodiments, the TLR modulator is a TLR8 modulator. In some embodiments, the TLR modulator is a TLR9 modulator. In some embodiments, the TLR modulator is a TLR3 agonist. In some embodiments, the TLR modulator is a TLR4 agonist. In some embodiments, the TLR modulator is a TLR7 agonist. In some embodiments, the TLR modulator is a TLR8 agonist. In some embodiments, the TLR modulator is a TLR9 agonist. In some embodiments, the TLR modulator is a TLR3 antagonist. In some embodiments, the TLR modulator is a TLR4 antagonist. In some embodiments, the TLR modulator is a TLR7 antagonist. In some embodiments, the TLR modulator is a TLR8 antagonist. In some embodiments, the TLR modulator is a TLR9 antagonist. In some embodiments, the TLR modulator described herein can modulate two or more TLRs. In some embodiments, the TLR modulator can activate one or more TLRs and inhibit one or more TLR. In some embodiments, the TLR modulator is a TLR9 modulator, such as KAPPAPROCT® or MONARSEN®. Some exemplary TLR modulators are described, for example, in WO2017059225A1.

In some embodiments, the immune modulating agent is a CpG-A or Cpg-B oligonucleotide as described in WO2017059225A1.

Cytosolic detection of pathogen-derived DNA requires signaling through TANK binding kinase 1 (TBK1) and its downstream transcription factor, IFN-regulatory factor 3 (IRF3). A transmembrane protein called STING (stimulator of IFN genes; also known as MITA, EMS, MPYS and TMEM173) functions as the signaling receptor for these cyclic purine dinucleotides, causing stimulation of the TBK1-IRF3 signalling axis and a STING-dependent type I interferon response. Thus in some embodiments, the immune modulating agent is a STING modulator. STING binds directly to cyclic diguanylate monophosphate, but not to other unrelated nucleotides or nucleic acids. Accordingly, in some embodiments, the STING modulator is a cyclic purine dinucleotide. Exemplary cyclic purine dinucleotides and STING modulators are described, for example, in US9549944B2, content of which is incorporated herein by reference in its entirety.

Additional exemplary immunosuppressants include, but are not limited to abatacept; adalimumab; adenosine receptor agonists; anakinra; aryl hydrocarbon receptor inhibitors; autophagy inhibitors, such as 3-Methyladenine; calcineurin inhibitors; calcineurin inhibitors, such as cyclosporine and tacrolimus; Caspase-1 inhibitors; certolizumab; cGAS inhibitors; corticosteroids; corticosteroids, such as predni sone, budesonide, prednisolone; cytokine inhibitors; cytokine receptor activators; cytokine receptor inhibitors; etanercept; glucocorticoids; golimumab; G-protein coupled receptor agonists; G-protein coupled receptor antagonists; histone deacetylase inhibitors; histone deacetylase inhibitors, such as Trichostatin A; IMDH inhibitors, such as azathioprine, leflunomide, and mycophenolate; infliximab; inhibitors of mitochondrial function, such as rotenone; ixekizumab; kinase inhibitors; Methylprednisolone; monoclonal antibodies, such as basiliximab, daclizumab, and muromonab; mTOR inhibitors, such as rapamycin or a rapamycin analog, sirolimus, and everolimus; natalizumab; NF-κβ inhibitors, such as 6Bio, Dexamethasone, TCPA-1, IKK VII; OTK3; oxidized ATPs, such as P2X receptor blockers; P38 inhibitors; peroxisome proliferator-activated receptor agonists; peroxi some proliferator-activated receptor antagonists; phosphatase inhibitors; phosphodiesterase inhibitors, such as phosphodiesterase 4 inhibitor (PDE4), such as Rolipram; PI3 KB inhibitors, such as TGX-221; prostaglandin E2 agonists (PGE2), such as Misoprostol; proteasome inhibitor I (PSI); proteasome inhibitors; retinoids; rituximab; secukinumab; statins; TGF-β receptor agonists; TGF-β signaling agents; Thymoglobulin; TLR9 antagonists; tocilizumab; ustekinumab; and vedolizumab. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol and triptolide, interleukins (e.g., IL-1, IL-10), cyclosporine A, siRNAs targeting cytokines or cytokine receptors and the like.

In some embodiments, the immune modulating agent is selected form the group consisting of 3-Methyladenine, 6-Bio, 6-mercaptopurine (6-MP, 6-thioguanine (6-TG), FK506, sanglifehrin A, abatacept, adalimumab, anakinra, aryl hydrocarbon receptor inhibitors, aspirin, autophagy inhibitors, azathioprine, basiliximab, budesonide, calcineurin inhibitors, Caspase-1 inhibitors, certolizumab, cGAS inhibitors, COX inhibitors, niflumic acid, cyclosporine, cytokine inhibitors, cytokine receptor activators, cytokine receptor inhibitors, daclizumab, Dexamethasone, estriol, etanercept, everolimus, glucocorticoids, golimumab, G-protein coupled receptor agonists, G-protein coupled receptor antagonists, histone deacetylase inhibitors, IDO, IKK VII, infliximab, interleukine-1, interleukine-10, ixekizumab, kinase inhibitors, leflunomide, Methylprednisolone, Misoprostol, muromonab, mycophenolate, mycophenolate mofetil (MMF), natalizumab, OTK3, oxidized ATPs, P2X receptor blockers, P38 inhibitors, peroxisome proliferator-activated receptor agonists, peroxisome proliferator-activated receptor antagonists, phosphatase inhibitors, PI3 KB inhibitors, prednisolone, prednisone, proteasome inhibitor I (PSI), proteasome inhibitors, rapamycin and rapamycin analogs, resveratrol, retinoids, rituximab, Rolipram, rotenone, salmeterol, secukinumab, sirolimus, statins, tacrolimus, TCPA-1, TGX-221, Thymoglobulin, TLR9 antagonists, tocilizumab, trichostatin A, triptolide, ustekinumab, vedolizumab, vitamin D3, and any combination thereof.

It is noted that any one of the immune modulating agent described herein can be used in with lipid nanoparticles, ceDNA vectors and/or compositions disclosed herein. For example, aimmune modulating agent can be used alone or combined with one or more (e.g., one, two, three, foru, five or more) other immune modulating agents described herein.

In some embodiments, the immune modulating agent is selected from the group consisting of aryl hydrocarbon receptor inhibitors; Caspase-1 inhibitors; cGAS inhibitors; cytokine inhibitors; G-protein coupled receptor agonists; G-protein coupled receptor antagonists; inhibitors of mitochondrial function; mTOR inhibitors; NF-κβ inhibitors; peroxisome proliferator-activated receptor agonists; phosphatase inhibitors; phosphodiesterase inhibitors, TGX-221; prostaglandin E2 agonists (PGE2); TLR9 antagonists; proteasome inhibitors; TGF-β receptor agonists and TGF-β signaling agents. One can use any one or more of the above agents alone or in combination with the LNP containing ceDNA.

Also provided herein is a pharmaceutical composition comprising the lipid nanoparticle and a pharmaceutically acceptable carrier or excipient.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

### Some Exemplary LNP Characteristics

Generally, the lipid nanoparticles of the invention have a mean diameter selected to provide an intended therapeutic effect. Accordingly, in some aspects, the lipid nanoparticle has a mean diameter from about 30 nm to about 150 nm, more typically from about 50 nm to about 150 nm, more typically about 60 nm to about 130 nm, more typically about 70 nm to about 110 nm, most typically about 85 nm to about 105 nm, and preferably about 100 nm. In some aspects, the disclosure provides for lipid particles that are larger in relative size to common nanoparticles and about 150 to 250 nm in size. Lipid nanoparticle particle size can be determined by quasi-elastic light scattering using, for example, a Malvern Zetasizer Nano ZS (Malvern, UK) system.

Depending on the intended use of the lipid particles, the proportions of the components can be varied and the delivery efficiency of a particular formulation can be measured using, for example, an endosomal release parameter (ERP) assay.

The ceDNA can be complexed with the lipid portion of the particle or encapsulated in the lipid position of the lipid nanoparticle. In some embodiments, the ceDNA can be fully encapsulated in the lipid position of the lipid nanoparticle, thereby protecting it from degradation by a nuclease, e.g., in an aqueous solution. In some embodiments, the ceDNA in the lipid nanoparticle is not substantially degraded after exposure of the lipid nanoparticle to a nuclease at 37° C. for at least about 20, 30, 45, or 60 minutes. In some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human.

In some aspects, the lipid nanoparticle formulation is a lyophilized powder.

In some embodiments, lipid nanoparticles are solid core particles that possess at least one lipid bilayer. In other embodiments, the lipid nanoparticles have a tion-bilayer structure, i.e., a non-lamellar (i.e., non-bilayer) morphology. Without limitations, the non-bilayer morphology can include, for example, three dimensional tubes, rods, cubic symmetries, etc. The non-Lamellar morphology (i.e., non-bilayer structure) of the lipid particles can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid nanoparticies (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense.

In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (20 1 0), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

Encapsulation of ceDNA in lipid particles can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated ceDNA, allowing it to interact with the membrane-impermeable dye. Encapsulation of ceDNA can be calculated as E=(I₀-I)/I₀, where I and Io refers to the fluorescence intensities before and after the addition of detergent.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and/or reduce therapeutically effective dosage frequency. In other aspects, the liposome formulation may simply include polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver a ceDNA with extended release or controlled release profile over a period of hours to weeks. In some related aspects, the liposome formulation may comprise aqueous chambers that are bound by lipid bilayers. In other related aspects, the liposome formulation encapsulates a ceDNA with additional components that undergo a physical transition at elevated temperature which releases the ceDNA over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (di stearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (di stearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and/or sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that is larger in relative size to common nanoparticles and about 150 to 250 nm in size. In some aspects, the liposome formulation is a lyophilized powder.

In some aspects, the disclosure provides for a liposome formulation that is made and loaded with ceDNA vectors obtained by the process of Example 1 or otherwise disclosed herein, by adding a weak base to a mixture having the isolated ceDNA outside the liposome. This addition increases the pH outside the liposomes to approximately 7.3 and drives the ceDNA into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In other aspects, the disclosure provides for a liposome formulation comprising phospholipids, lecithin, phosphatidylcholine and phosphatidylethanolamine.

The ceDNA disclosed herein can be incorporated into pharmaceutical compositions suitable for administration to a subject for in vivo delivery to cells, tissues, or organs of the subject. Typically, the pharmaceutical composition comprises the ceDNA disclosed herein and a pharmaceutically acceptable carrier. For example, ceDNA vectors of the invention can be incorporated into a pharmaceutical composition suitable for a desired route of therapeutic administration (e.g., parenteral administration). Passive tissue transduction via high pressure intravenous or intraarterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable for high ceDNA vector concentration. Sterile injectable solutions can be prepared by incorporating the ceDNA vector compound in the required amount in an appropriate buffer with one or a combination of ingredients enumerated above, as required, followed by filtered sterilization.

A ceDNA vector as disclosed herein can be incorporated into a pharmaceutical composition suitable for topical, systemic, intra-amniotic, intrathecal, intracranial, intraarterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutically active compositions comprising a ceDNA vector can be formulated to deliver a transgene in the nucleic acid to the cells of a recipient, resulting in the therapeutic expression of the transgene therein. The composition can also include a pharmaceutically acceptable carrier.

The compositions and vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment, amelioration, prevention of disease states in a mammalian subject. The transgene can be transferred to (e.g., expressed in) a subject in a clinical setting in a sufficient amount to treat a disease associated with reduced expression, lack of expression or dysfunction of the gene. In some embodiments the transgene can be transferred to (e.g., expressed in) a subject in a sufficient amount to treat a disease associated with increased expression, activity of the gene product, or inappropriate upregulation of a gene that the transgene suppresses or otherwise causes the expression of which to be reduced.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

A ceDNA vector described herein can be administered to an organism for transduction of cells in vivo.

Generally, administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Exemplary modes of administration of the ceDNA vector disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the ceDNA vector can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the ceDNA vector can also be to a tumor (e.g., in or near a tumor or a lymph node). The most suitable route in any given case will depend on the nature and severity of the condition being treated, ameliorated, and/or prevented and on the nature of the particular ceDNA vector that is being used. Additionally, ceDNA permits one to administer more than one transgene in a single vector, or multiple ceDNA vectors (e.g. a ceDNA cocktail).

Administration of the ceDNA vector disclosed herein to skeletal muscle includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA as disclosed herein vector can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In particular embodiments, the ceDNA vector as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In embodiments, the ceDNA vector as disclosed herein can be administered without employing “hydrodynamic” techniques.

Administration of the ceDNA vector as disclosed herein to cardiac muscle includes administration to the left atrium, right atrium, left ventricle, right ventricle and/or septum. The ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In some embodiments, a ceDNA vector according to the present invention is administered to skeletal muscle, diaphragm muscle and/or cardiac muscle (e.g., to treat, ameliorate, and/or prevent muscular dystrophy or heart disease (e.g., PAD or congestive heart failure).

A ceDNA vector according to the present invention can be administered to the CNS (e.g., to the brain or to the eye). The ceDNA vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The ceDNA vector may also be administered to different regions of the eye such as the retina, cornea and/or optic nerve. The ceDNA vector may be delivered into the cerebrospinal fluid (e.g., by lumbar puncture). The ceDNA vector may further be administered intravascularly to the CNS in situations in which the blood-brain barrier has been perturbed (e.g., brain tumor or cerebral infarct).

Thhe ceDNA vector can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the ceDNA vector is administered in a liquid formulation by direct injection (e.g., stereotactic injection) to the desired region or compartment in the CNS. In other embodiments, the ceDNA vector can be provided by topical application to the desired region or by intra-nasal administration of an aerosol formulation. Administration to the eye may be by topical application of liquid droplets. As a further alternative, the ceDNA vector can be administered as a solid, slow-release formulation (see, e.g., U.S. Pat. No. 7,201,898). In yet additional embodiments, the ceDNA vector can used for retrograde transport to treat, ameliorate, and/or prevent diseases and disorders involving motor neurons (e.g., amyotrophic lateral sclerosis (ALS); spinal muscular atrophy (SMA), etc.). For example, the ceDNA vector can be delivered to muscle tissue from which it can migrate into neurons.

### Ex Vivo Treatment

In some embodiments, cells are removed from a subject, a ceDNA vector is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, the ceDNA vector can encode a transgene (sometimes called a heterologous nucleotide sequence) that is any polypeptide that is desirably produced in a cell in vitro, ex vivo, or in vivo. For example, in contrast to the use of the ceDNA vectors in a method of treatment, in some embodiments the ceDNA vectors may be introduced into cultured cells and the expressed gene product isolated therefrom, e.g., for the production of antigens or vaccines.

The ceDNA vectors can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

In some embodiments, a transgene present in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

In some embodiments, the ceDNA vector expresses the transgene in a subject host cell. In some embodiments, the subject host cell is a human host cell, including, for example blood cells, stem cells, hematopoietic cells, CD34⁺cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell

One aspect of the technology described herein relates to a method of delivering a transgene to a cell. Typically, for in vitro methods, the ceDNA vector may be introduced into the cell using the methods as disclosed herein, as well as other methods known in the art. ceDNA vectors disclosed herein are preferably administered to the cell in a biologically-effective amount. If the ceDNA vector is administered to a cell in vivo (e.g., to a subject), a biologically-effective amount of the ceDNA vector is an amount that is sufficient to result in transduction and expression of the transgene in a target cell.

### Dose Ranges

In vivo and/or in vitro assays can optionally be employed to help identify optimal dosage ranges for use. The precise dose to be employed in the formulation will also depend on the route of administration, and the seriousness of the condition, and should be decided according to the judgment of the person of oridinary skill in the art and each subject's circumstances. Effective doses can be extrapolated from dose-response curves derived from in vitro or animal model test systems.

A ceDNA vector is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vector required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a ceDNA vector dose range to treat a subject having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1μg to 100 g of the ceDNA vector. If exosomes or microparticles are used to deliver the ceDNA vector, then a therapeutically effective dose can be determined experimentally, but is expected to deliver from 1 μg to about 100 g of vector.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of a ceDNA vector to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

Treatment can involve administration of a single dose or multiple doses. In particular embodiments, more than one administration (e.g., two, three, four or more administrations) may be employed to achieve the desired level of gene expression over a period of various intervals, e.g., daily, weekly, monthly, yearly, etc. In some embodiments, more than one dose can be administered to a subject; in fact multiple doses can be administered as needed, because the ceDNA vector does not elicit an anti-capsid host immune response due to the absence of a viral capsid. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a ceDNA vector as described by the disclosure (i.e., the absence of capsid components) allows the ceDNA vector to be administered to a host on multiple occasions. In some embodiments, the number of occasions in which a heterologous nucleic acid is delivered to a subject is in a range of 2 to 10 times (e.g., 2, 3, 4, 5, 6, 7, 8, 9, or 10 times). In some embodiments, a ceDNA vector is delivered to a subject more than 10 times.

In some embodiments, a dose of a ceDNA vector is administered to a subject no more than once per calendar day (e.g., a 24-hour period). In some embodiments, a dose of a ceDNA vector is administered to a subject no more than once per 2, 3, 4, 5, 6, or 7 calendar days. In some embodiments, a dose of a ceDNA vector is administered to a subject no more than once per calendar week (e.g., 7 calendar days). In some embodiments, a dose of a ceDNA vector is administered to a subject no more than bi-weekly (e.g., once in a two calendar week period). In some embodiments, a dose of a ceDNA vector is administered to a subject no more than once per calendar month (e.g., once in 30 calendar days). In some embodiments, a dose of a ceDNA vector is administered to a subject no more than once per six calendar months. In some embodiments, a dose of a ceDNA vector is administered to a subject no more than once per calendar year (e.g., 365 days or 366 days in a leap year).

### Unit Dosage Forms

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect

### Preparation of Lipid Nanoparticles

Lipid nanoparticles can form spontaneously upon mixing of ceDNA and the lipid(s). Depending on the desired particle size distribution, the resultant nanoparticle mixture can be extruded through a membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration.

Generally, lipid nanoparticles can be formed by any method known in the art including, For example, the lipid nanoparticles can be prepared by the methods described, for example, in US2013/0037977, MO10/0015218, US2013/0156845, US2013/0164400, US2012/0225129, and US2010/0130588, content of each of which is incorporated herein by reference in its entirety. In some embodiments, lipid nanoparticles can be prepared using a continuous mixing method, a direct dilution process, or an in-line dilution process. The processes and apparatuses for apparatuses for preparing lipid nanoparticles using direct dilution and in-line dilution processes are described in US2007/0042031, the content of which is incorporated herein by reference in its entirety. The processes and apparatuses for preparing lipid nanoparticles using step-wise dilution processes are described in US2004/0142025, the content of which is incorporated herein by reference in its entirety.

In one non-limiting example, the lipid nanoparticles can be prepared by an impinging jet process. Generally, the particles are formed by mixing lipids dissolved in alcohol (e.g., ethanol) with ceDNA dissolved in a buffer, e.g., a citrate buffer, a sodium acetate buffer, a sodium acetate and magnesium chloride buffer, a malic acid buffer, a malic acid and sodium chloride buffer, or a sodium citrate and sodium chloride buffer. The mixing ratio of lipids to ceDNA can be about 45-55% lipid and about 65-45% ceDNA.

The lipid solution can contain an ionizable lipid, a non-cationic lipid (e.g., a phospholipid, such as DPSC), PEG or PEG conjugated molecule (e.g., PEG-lipid), and a sterol (e.g., cholesterol) at a total lipid concentration of 5-30 mg/mL, more likely 5-15 mg/mL, most likely 9-12 mg/mL in an alcohol, e.g., in ethanol.

In the lipid solution, mol ratio of the lipids can range from about 25-98% for the cationic lipid, preferably about 35-65%; about 0-15% for the non-ionic lipid, preferably about 0-12%; about 0-15% for the PEG or PEG conjugated molecule, preferably about 1-6%; and about 0-75% for the sterol, preferably about 30-50%.

The ceDNA solution can comprise the ceDNA at a concentration range from 0.3 to 1.0 mg/mL, preferably 0.3-0.9 mg/mL in buffered solution, with pH in the range of 3.5-5.

For forming the LNPs, in one exemplary but nonlimiting embodiment, the two liquids are heated to a temperature in the range of about 15-40° C., preferably about 30-40° C., and then mixed, for example, in an impinging jet mixer, instantly forming the LNP. The mixing flow rate can range from 10-600 ml/min. The tube ID can have a range from 0.25 to 1.0 mm and a total flow rate from 10-600 mL/min. The combination of flow rate and tubing ID can have the effect of controlling the particle size of the LNPs between 30 and 200 nm. The solution can then be mixed with a buffered solution at a higher pH with a mixing ratio in the range of 1:1 to 1:3 vol:vol, preferably about 1:2 vol:vol. If needed this buffered solution can be at a temperature in the range of 15-40° C. or 30-40° C. The mixed LNPs can then undergo an anion exchange filtration step. Prior to the anion exchange, the mixed LNPs can be incubated for a period of time, for example 30mins to 2 hours. The temperature during incubating can be in the range of 15-40° C. or 30-40° C. After incubating the solution is filtered through a filter, such as a 0.8 μm filter, containing an anion exchange separation step. This process can use tubing IDs ranging from 1 mm ID to 5 mm ID and a flow rate from 10 to 2000 mL/min.

After formation, the LNPs can be concentrated and diafiltered via an ultrafiltration process where the alcohol is removed and the buffer is exchanged for the final buffer solution, for example, phosphate buffered saline (PBS) at about pH 7, e.g., about pH 6.9, about p1-I 7.0, about pH 7.1, about pH 7.2, about pH 7.3, or about pH 7.4.

The ultrafiltration process can use a tangential flow filtration format (TFF) using a membrane nominal molecular weight cutoff range from 30-500 KD. The membrane format is hollow fiber or flat sheet cassette. The TFF processes with the proper molecular weight cutoff can retain the LNP in the retentate and the filtrate or permeate contains the alcohol; citrate buffer and final buffer wastes. The TFF process is a multiple step process with an initial concentration to a ceDNA concentration of 1 -3 mg/mL. Following concentration, the LNPs solution is diafiltered against the final buffer for 10 -20 volumes to remove the alcohol and perform buffer exchange. The material can then be concentrated an additional 1-3 fold. The concentrated LNP solution can be sterile filtered. ceDNA

In various embodiments, the ceDNA vectors are capsid-free, linear duplex DNA molecules formed from a continuous strand of complementary DNA with covalently-closed ends (linear, continuous and non-encapsidated structure), which comprise a 5′ inverted terminal repeat (ITR) sequence and a 3′ ITR sequence that are different, or asymmetrical with respect to each other. At least one of the ITRs comprises a functional terminal resolution site and a replication protein binding site (RPS) (sometimes referred to as a replicative protein binding site), e.g. a Rep binding site. Generally, the ceDNA vector contains at least one modified AAV inverted terminal repeat sequence (ITR), i.e., a deletion, insertion, and/or substitution with respect to the other ITR, and an expressible transgene.

In one embodiment, at least one of the ITRs is an AAV ITR, e.g. a wild type AAV ITR. In one embodiment, at least one of the ITRs is a modified ITR relative to the other ITR—that is, the ceDNA comprises ITRs that are asymmetric relative to each other. In one embodiment, at least one of the ITRs is a non-functional ITR.

In some embodiments, the ceDNA vector comprises: (1) an expression cassette comprising a cis-regulatory element, a promoter and at least one transgene; or (2) a promoter operably linked to at least one transgene, and (3) two self-complementary sequences, e.g., ITRs, flanking said expression cassette, wherein the ceDNA vector is not associated with a capsid protein. In some embodiments, the ceDNA vector comprises two self-complementary sequences found in an AAV genome, where at least one comprises an operative Rep-binding element (RBE) and a terminal resolution site (trs) of AAV or a functional variant of the RBE, and one or more cis-regulatory elements operatively linked to a transgene. In some embodiments, the ceDNA vector comprises additional components to regulate expression of the transgene, for example, regulatory switches for controlling and regulating the expression of the transgene, and can include a regulatory switch which is a kill switch to enable controlled cell death of a cell comprising a ceDNA vector.

In some embodiments, the two self-complementary sequences can be ITR sequences from any known parvovirus, for example a dependovirus such as AAV (e.g., AAV1-AAV12). Any AAV serotype can be used, including but not limited to a modifed AAV2 ITR sequence, that retains a Rep-binding site (RBS) such as 5′-GCGCGCTCGCTCGCTC-3′ (SEQ ID NO: 531) and a terminal resolution site (trs) in addition to a variable palindromic sequence allowing for hairpin secondary structure formation. In some embodiments, an ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects a synthetic ITR may interact preferentially with a wildtype Rep or a Rep of a specific serotype, or in some instances will not be recognized by a wild-type Rep and be recognized only by a mutated Rep. In some embodiments, the ITR is a synthetic ITR sequence that retains a functional Rep-binding site (RBS) such as 5′-GCGCGCTCGCTCGCTC-3′ (SEQ ID NO: 531) and a terminal resolution site (TRS) in addition to a variable palindromic sequence allowing for hairpin secondary structure formation. In some examples, a modified ITR sequence retains the sequence of the RBS, trs and the structure and position of a Rep binding element forming the terminal loop portion of one of the ITR hairpin secondary structure from the corresponding sequence of the wild-type AAV2 ITR. Exemplary ITR sequences for use in the ceDNA vectors are disclosed in Tables 2-9, 10A and 10B, SEQ ID NO: 2, 52, 101-449 and 545-547, and the partial ITR sequences shown in FIGS. 26A-26B of PCT application No. PCT/US18/49996, filed Sep. 7, 2018. In some embodiments, a ceDNA vector can comprise an ITR with a modification in the ITR corresponding to any of the modifications in ITR sequences or ITR partial sequences shown in any one or more of Tables 2, 3, 4, 5, 6, 7, 8, 9, 10A and 10B PCT application No. PCT/US18/49996, filed Sep. 7, 2018.

In some embodiments, the closed-ended DNA vector comprises a promoter operably linked to a transgene, where the ceDNA is devoid of capsid proteins and is: (a) produced from a ceDNA-plasmid that encodes a mutated right side AAV2 ITR having the same number of intramolecularly duplexed base pairs as SEQ ID NO:2 or a mutated left side AAV2 ITR having the same number of intramolecularly duplexed base pairs as SEQ ID NO:51 in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions in Example 1.

The ceDNA vectors are obtainable by a number of means that would be known to the ordinarily skilled artisan after reading this disclosure. For example, the capsid free non-viral DNA vector can be obtained from a plasmid (referred to herein as a “ceDNA-plasmid”) comprising a polynucleotide expression construct template comprising in this order: a first 5′ inverted terminal repeat (e.g. AAV ITR); an expression cassette; and a 3′ ITR (e.g. AAV ITR), where at least one of the 5′ and 3′ ITR is a modified ITR, or where when both the 5′ and 3′ ITRs are modified, they have different modifications from one another and are not the same sequence. Without limitations, a polynucleotide expression construct template used for generating the ceDNA vectors can be a ceDNA-plasmid, a ceDNA-bacmid, and/or a ceDNA-baculovirus. In some embodiments, the ceDNA-plasmid comprises a restriction cloning site (e.g. SEQ ID NO: 7) operably positioned between the ITRs where an expression cassette comprising e.g., a promoter operatively linked to a transgene, e.g., a reporter gene and/or a therapeutic gene) can be inserted.

For example, the non-viral capsid free DNA vectors can be produced in permissive host cells from an expression construct (e.g., a plasmid, a bacmid, a baculovirus, or an integrated cell-line) containing a heterologous gene positioned between two different inverted terminal repeat (ITR) sequences. At least one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g., self-complementary, over at least a portion of the molecule, such as the expression cassette. e.g. ceDNA is not a double stranded circular molecular. In some embodiments, the ceDNA vector has covalently closed ends, and thus is resistant to exonuclease digestion (e.g. exonuclease I or exonuclease III), e.g. for over an hour at 37° C.

In some embodiments, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR and the second ITR are asymmetric with respect to each other—that is, they are different from one another. As an exemplary embodiment, the first ITR can be a wild-type ITR and the second ITR can be a mutated or modified ITR. In some embodiments, the first ITR can be a mutated or modified ITR and the second ITR a wild-type ITR. In another embodiment, the first ITR and the second ITR are both modified but are different sequences, or have different modifications, or are not identical modified ITRs. Stated differently, the ITRs are asymmetric in that any changes in one ITR are not reflected in the other ITR; or alternatively, where the ITRs are different with respect to each other.

In some embodiments, an expression cassette is located between two ITRs comprised in the following order with one or more of: a promoter operably linked to a transgene, a posttranscriptional regulatory element, and a polyadenylation and termination signal. In one embodiment, the promoter is regulatable-inducible or repressible. The promoter can be any sequence that facilitates the transcription of the transgene. In one embodiment the promoter is a CAG promoter (e.g. SEQ ID NO: 03), or variation thereof. The posttranscriptional regulatory element is a sequence that modulates expression of the transgene, as a non-limiting example, any sequence that creates a tertiary structure that enhances expression of the transgene.

Generally, the ceDNA vectors have no packaging constraints imposed by the limiting space within the viral capsid. ceDNA vectors as opposed to encapsulated AAV genomes represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

In some embodiments, the first ITR can be a mutated or modified ITR and the second ITR a wild-type ITR. In another embodiment, the first ITR and the second ITR are both modified but are different sequences, or have different modifications, or are not identical modified ITRs. Stated differently, the ITRs are asymmetric in that any changes in one ITR are not reflected in the other ITR; or alternatively, where the ITRs are different with respect to each other. Exemplary ITRs in the ceDNA vector and for use to generate a ceDNA-plasmid are disclosed in PCT application No. PCT/US18/49996, filed Sep. 7, 2018.

While the ITRs exemplified in the specification and Examples herein are AAV2 ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvoviris (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148).

The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

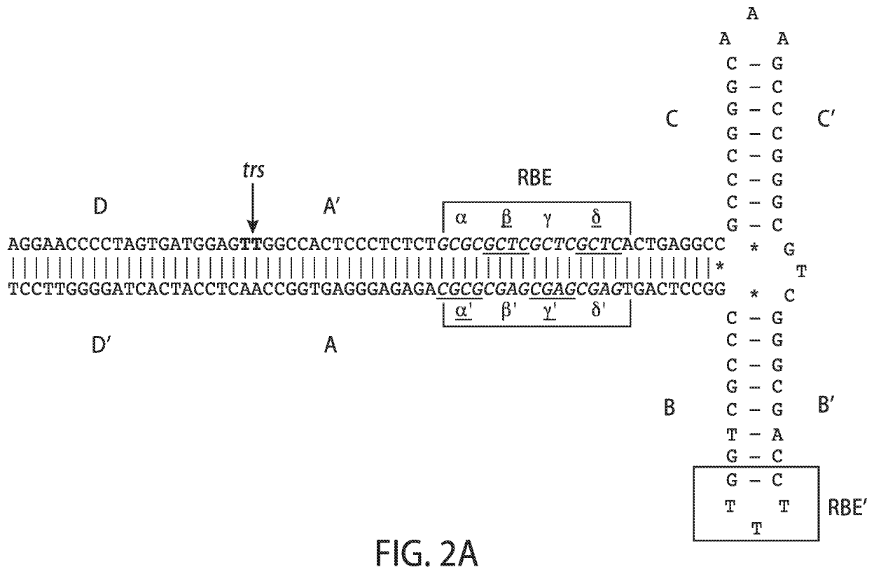

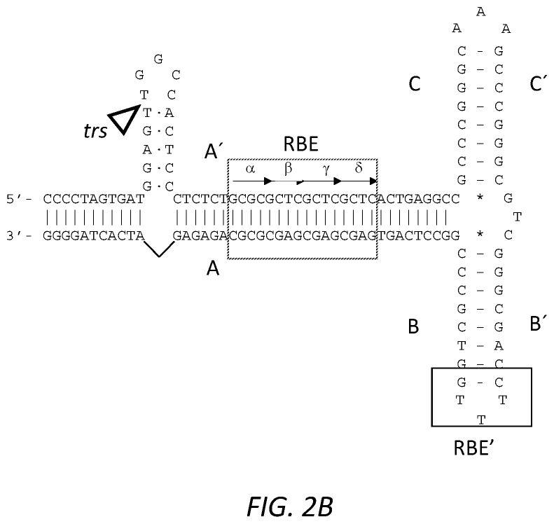

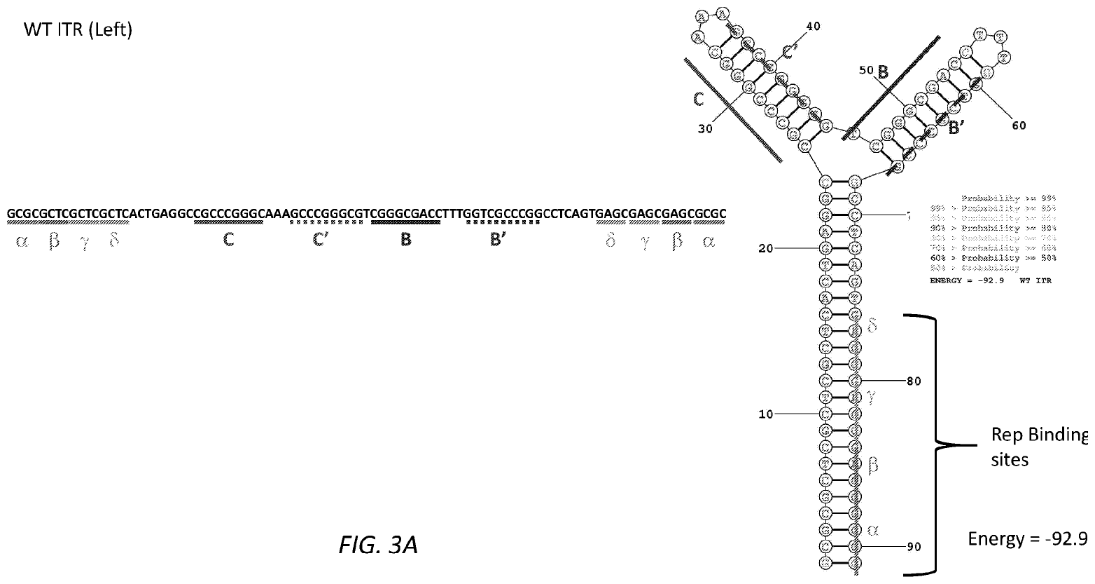

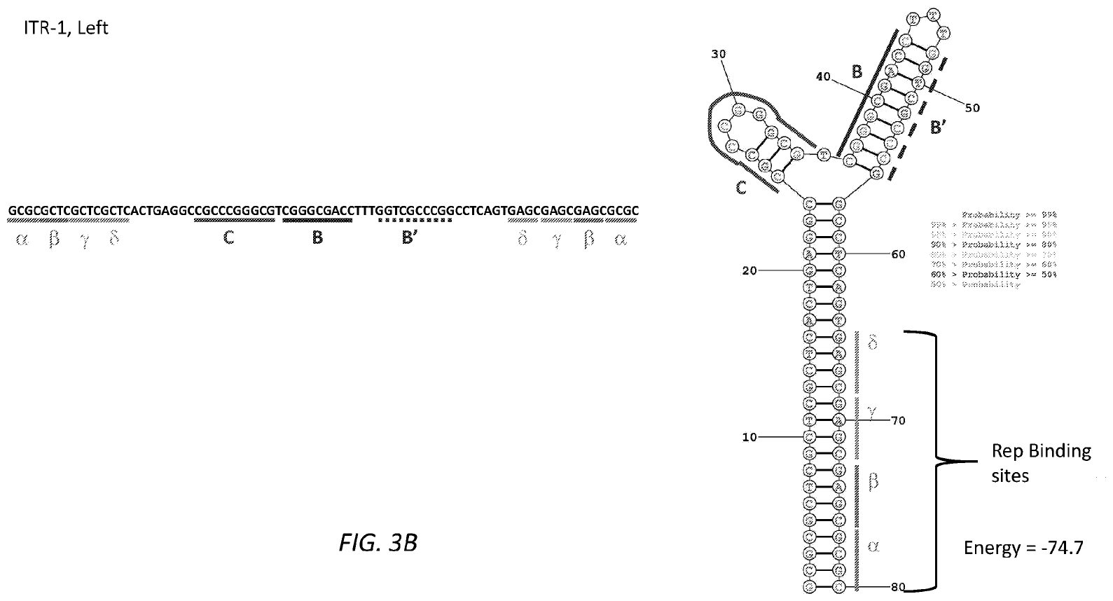

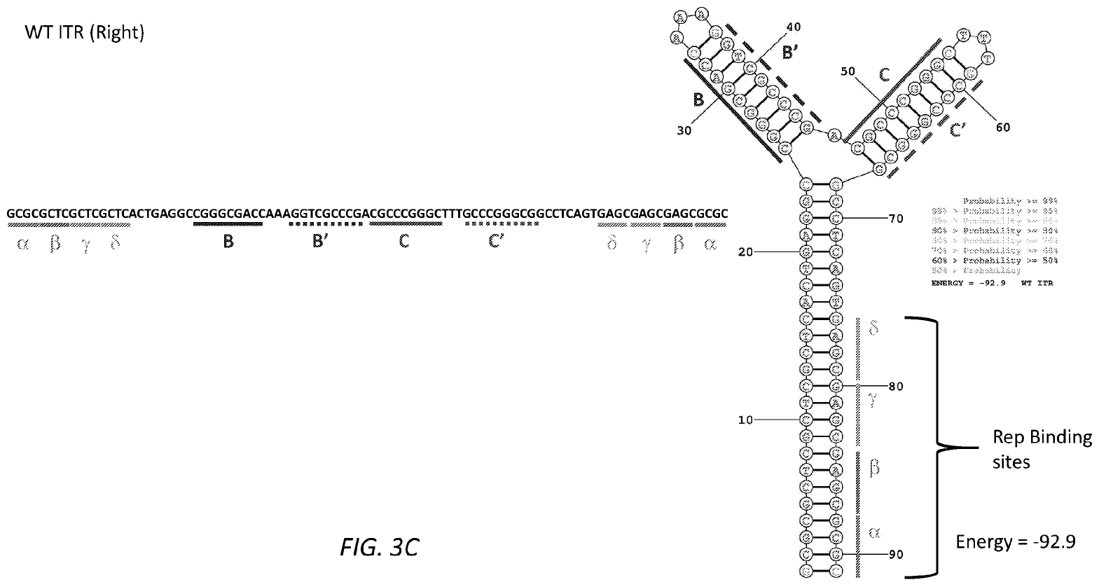

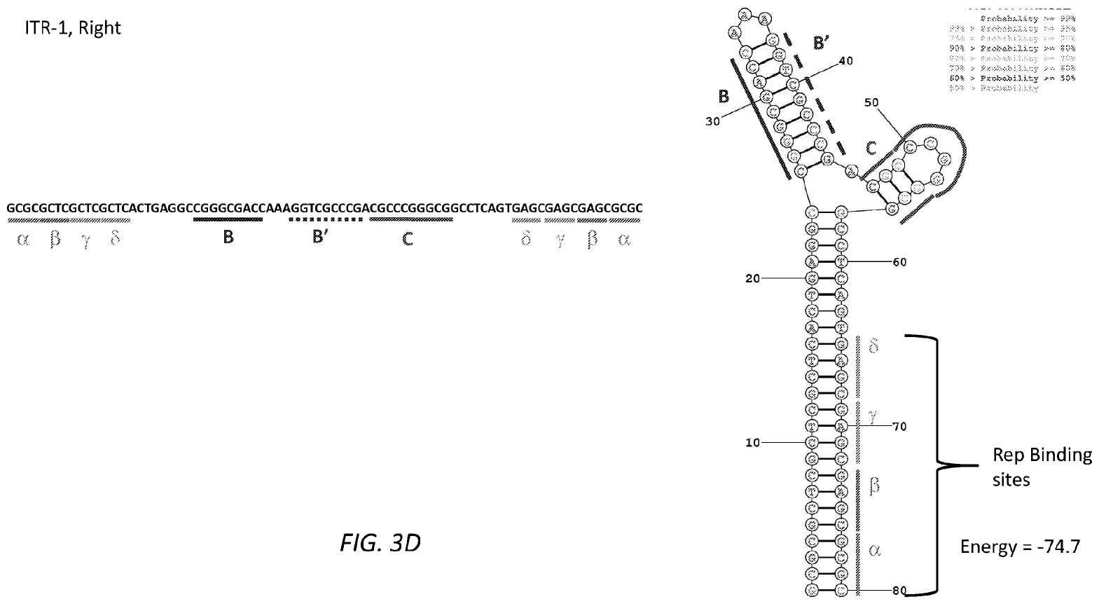

An ordinarily skilled artisan is aware that ITR sequences have a common structure of a double-stranded Holliday junction, which typically is a T-shaped or Y-shaped hairpin structure (see e.g., FIGS. 2A and 3A), where each ITR is formed by two palindromic arms or loops (B-B′ and C-C′) embedded in a larger palindromic arm (A-A′), and a single stranded D sequence, (where the order of these palindromic sequences defines the flip or flop orientation of the ITR), one can readily determine corresponding modified ITR sequences from any AAV serotype for use in a ceDNA vector or ceDNA-plasmid based on the exemplary AAV2 ITR sequences provided herein. See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. Exemplary specific alterations and mutations in the ITRs are described in detail in PCT application No. PCT/US18/49996, filed Sep. 7, 2018. For clarity, in the context of ITRs, “altered” or “mutated” indicates that nucleotides have been inserted, deleted, and/or substituted relative to the wild-type, reference, or original ITR sequence, and can be altered relative to the other flanking ITR in a ceDNA vector having two flanking ITRs. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAVS preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR. For example, it is selected from the group consisting of: SEQ ID NO:2 and SEQ ID NO:52. In one embodiment of each of these aspects, the vector polynucleotide comprises a pair of ITRs, selected from the group consisting of: SEQ ID NO:1 and SEQ ID NO:52; and SEQ ID NO:2 and SEQ ID NO:51. In one embodiment of each of these aspects, the vector polynucleotide or the non-viral, capsid-free DNA vectors with covalently-closed ends comprises a pair of different ITRs selected from the group consisting of: SEQ ID NO:101 and SEQ ID NO:102; SEQ ID NO:103, and SEQ ID NO:104, SEQ ID NO:105, and SEQ ID NO:106; SEQ ID NO:107, and SEQ ID NO:108; SEQ ID NO:109, and SEQ ID NO:110; SEQ ID NO:111, and SEQ ID NO:112; SEQ ID NO:113 and SEQ ID NO:114; and SEQ ID NO:115 and SEQ ID NO:116. In some embodiments, a modified ITR is selected from any of SEQ ID NOS: 2, 52, 63, 64, 101-499, and disclosed in PCT application No. PCT/US18/49996, filed September 7 2018. In some embodiments, a ceDNA vector does not not have a modified ITR selected from any sequence consisting of, or consisting essentially of: SEQ ID NOs:500-529, as provided herein. In some embodiments, a ceDNA vector does not have an ITR that is selected from any sequence selected from SEQ ID NOs:500-529.

In some embodiments, ceDNA can form an intramolecular duplex secondary structure. The secondary structure of the first ITR and the asymmetric second ITR are exemplified in the context of wild-type ITRs (see, e.g., FIGS. 2A, 3A and 3C) and modified ITR structures (see e.g., FIGS. 2B, 3B and 3D). Secondary structures are inferred or predicted based on the ITR sequences of the plasmid used to produce the ceDNA vector.

In some embodiments, the left ITR of the ceDNA vector is modified or mutated with respect to a wild type (wt) AAV ITR structure, and the right ITR is a wild type ITR. In one embodiment, the right ITR of the ceDNA vector is modified with respect to a wild type AAV ITR structure, and the left ITR is a wild type AAV ITR. In such embodiments, a modification of the ITR (e.g., the left or right ITR) can be generated by a deletion, an insertion, or substitution of one or more nucleotides from the wild type ITR derived from the AAV genome.

The ITRs used herein can be resolvable and non-resolvable, and selected for use in the ceDNA vectors are preferably AAV sequences, with serotypes 1, 2, 3, 4, 5, 6, 7, 8 and 9 being preferred. Resolvable AAV ITRs do not require a wild-type ITR sequence (e.g., the endogenous or wild-type AAV ITR sequence may be altered by insertion, deletion, truncation and/or missense mutations), as long as the terminal repeat mediates the desired functions, e.g., replication, virus packaging, integration, and/or provirus rescue, and the like. Typically, but not necessarily, the TRs are from the same AAV serotype, e.g., both ITR sequences of the ceDNA vector are from AAV2. The ITRs may be synthetic sequences that function as AAV inverted terminal repeats While not necessary, the ITRs can be from the same parvovirus, e.g., both ITR sequences are from AAV2.

In some embodiments, at least one of the ITRs is a defective ITR with respect to Rep binding and/or Rep nicking. In one embodiment, the defect is at least 30% relative to a wild type reduction ITR, in other embodiments it is at least 35% . . . , 50% . . . , 65% . . . , 75% . . . , 85% . . . , 90% . . . , 95% . . . , 98% . . . , or completely lacking in function or any point in-between. The host cells do not express viral capsid proteins and the polynucleotide vector template is devoid of any viral capsid coding sequences. In one embodiment, the polynucleotide vector templates and host cells that are devoid of AAV capsid genes and the resultant protein also do not encode or express capsid genes of other viruses. In addition, in a particular embodiment, the nucleic acid molecule is also devoid of AAV Rep protein coding sequences

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE', and a terminal resolution sire (trs).

More specifically, the ability of a structural element to functionally interact with a particular large Rep protein can be altered by modifying the structural element. For example, the nucleotide sequence of the structural element can be modified as compared to the wild-type sequence of the ITR. In one embodiment, the structural element (e.g., A arm, A′ arm, B arm, B′ arm, C arm, C′ arm, D arm, RBE, RBE', and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

In some embodiments, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element.

In some embodiments, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In some embodiments, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In some embodiments, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In some embodiments, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

FIGS. 2A and 2B show one possible mechanism for the operation of a trs site within a wild type ITR structure portion of a ceDNA vector. In some embodiments, the ceDNA vector contains one or more functional ITR polynucleotide sequences that comprise a Rep-binding site (RBS; 5′-GCGCGCTCGCTCGCTC-3′ for AAV2, SEQ ID NO: 531) and a terminal resolution site (TRS; 5′-AGTT, SEQ ID NO: 46). In some embodiments, at least one ITR (wt or modified ITR) is functional. In alternative embodiments, where a ceDNA vector comprises two modified ITRs that are different or asymmetrical to each other, at least one modified ITR is functional and at least one modified ITR is non-functional.

In some embodiments, the modified ITR (e.g., the left or right ITR) of the ceDNA vector described herein has modifications within the loop arm, the truncated arm, or the spacer. Exemplary sequences of ITRs having modifications within the loop arm, the truncated arm, or the spacer are listed in Tables 2-9, 10A and 10B of PCT application No. PCT/US18/49996, filed Sep. 7, 2018.

The ceDNA vectors can be produced from expression constructs that further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the ceDNA vector comprises additional components to regulate expression of the transgene, for example, regulatory switches as described in PCT application No. PCT/US18/49996, filed Sep. 7, 2018, to regulate the expression of the transgene or a kill switch, which can kill a cell comprising the ceDNA vector.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 8) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 25 and SEQ ID NO: 26. The expression cassettes can include a poly-adenylation sequence known in the art or a variation thereof, such as a naturally occurring sequence isolated from bovine BGHpA (e.g., SEQ ID NO: 74) or a virus SV40pA (e.g., SEQ ID NO: 10), or a synthetic sequence (e.g., SEQ ID NO: 27). Some expression cassettes can also include SV40 late polyA signal upstream enhancer (USE) sequence. The, USE can be used in combination with SV40pA or heterologous poly-A signal.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene or nucleic acid in the range of 500 to 50,000 nucleotides in length. In some embodiments, the expression cassette can comprise a transgene or nucleic acid in the range of 500 to 75,000 nucleotides in length. In some embodiments, the expression cassette can comprise a transgene or nucleic acid is in the range of 500 to 10,000 nucleotides in length. In some embodiments, the expression cassette can comprise a transgene or nucleic acid is in the range of 1000 to 10,000 nucleotides in length. In some embodiments, the expression cassette can comprise a transgene or nucleic acid is in the range of 500 to 5,000 nucleotides in length. The ceDNA vectors do not have the size limitations of encapsidated AAV vectors, thus enable delivery of a large-size expression cassette to provide efficient expression of transgenes. In some embodiments, the ceDNA vector is devoid of prokaryote-specific methylation. In some embodiments, the expression cassette length in the 5′ to 3′ direction is greater than the maximum length known to be encapsidated in an AAV virion. In some embodiments, length is greater than 4.6 kb, or greater than 5 kb, or greater than 6 kb, or greater than 7 kb.

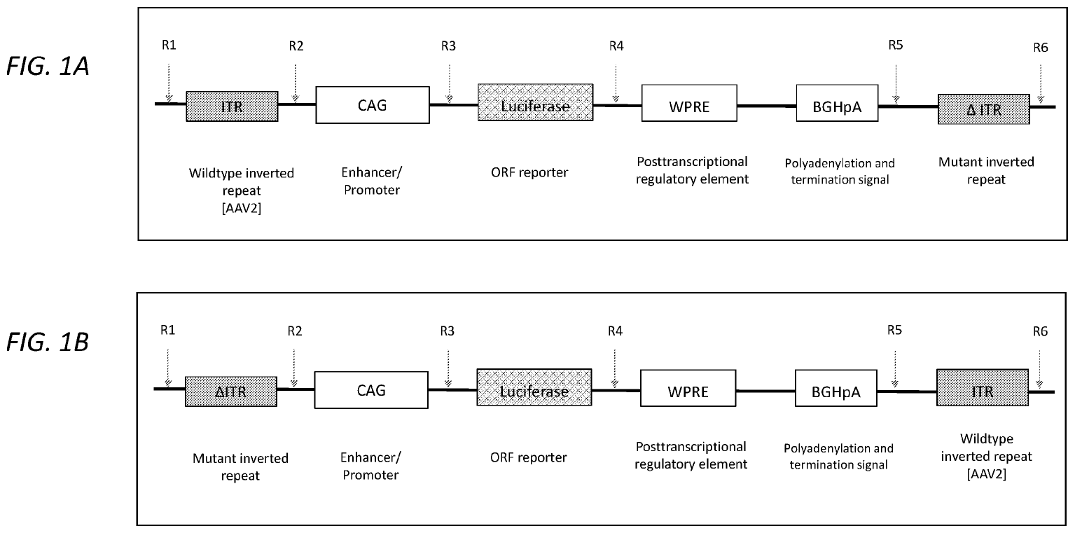

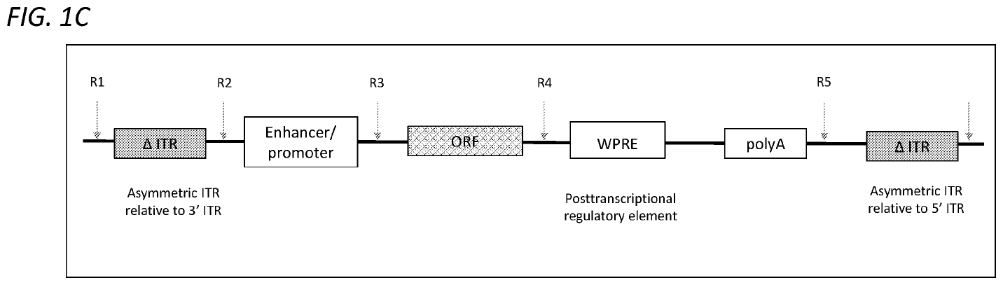

FIGS. 1A-1C show schematics of nonlimiting, exemplary ceDNA vectors, or the corresponding sequence of ceDNA plasmids. ceDNA vectors are capsid-free and can be obtained from a plasmid encoding in this order: a first ITR, expressible transgene cassette and a second ITR, where at least one of the first and/or second ITR sequence is mutated with respect to the corresponding wild type AAV2 ITR sequence. The expressible transgene cassette preferably includes one or more of, in this order: an enhancer/promoter, an ORF reporter (transgene), a post-transcription regulatory element (e.g., WPRE), and a polyadenylation and termination signal (e.g., BGH polyA).

The expression cassette can comprise any transgene of interest. Transgenes of interest include but are not limited to, nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the transgenes in the expression cassette encodes one or more polypeptides, peptides, ribozymes, peptide nucleic acids, siRNAs, RNAis, antisense oligonucleotides, antisense polynucleotides, antibodies, antigen binding fragments, or any combination thereof. In some embodiments, the transgene is a therapeutic gene, or a marker protein. In some embodiments, the transgene is an agonist or antagonist. In some embodiments, the antagonist is a mimetic or antibody, or antibody fragment, or antigen-binding fragment thereof, e.g., a neutralizing antibody or antibody fragment and the like. In some embodiments, the transgene encodes an antibody, including a full-length antibody or antibody fragment, as defined herein. In some embodiments, the antibody is an antigen-binding domain or an immunoglobulin variable domain sequence, as that is defined herein.

In particular, the transgene can encode one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder.

There are many structural features of ceDNA vectors that differ from plasmid-based expression vectors. ceDNA vectors may possess one or more of the following features the lack of original (i.e. not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, of the eukaryotic origin (i.e., they are produced in eukaryotic cells), and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a nonlimiting example in a promoter or enhancer region. Another important feature distinguishing ceDNA vectors from plasmid expression vectors is that ceDNA vectors are single-strand linear DNA having closed ends, while plasmids are always double-stranded DNA.

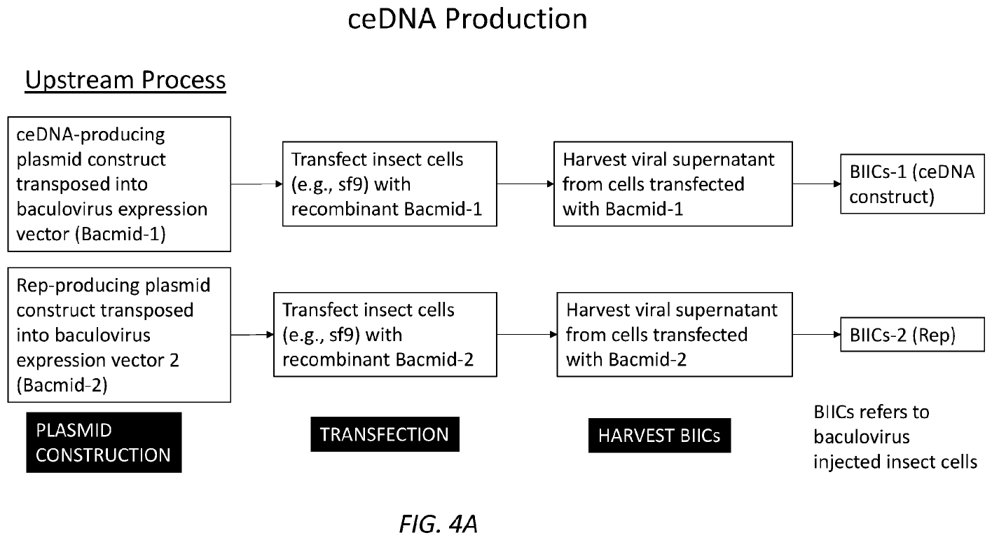

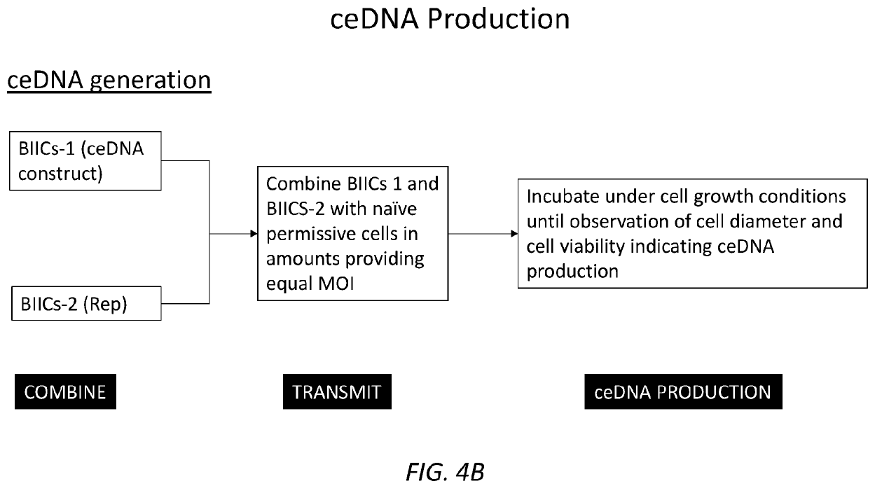

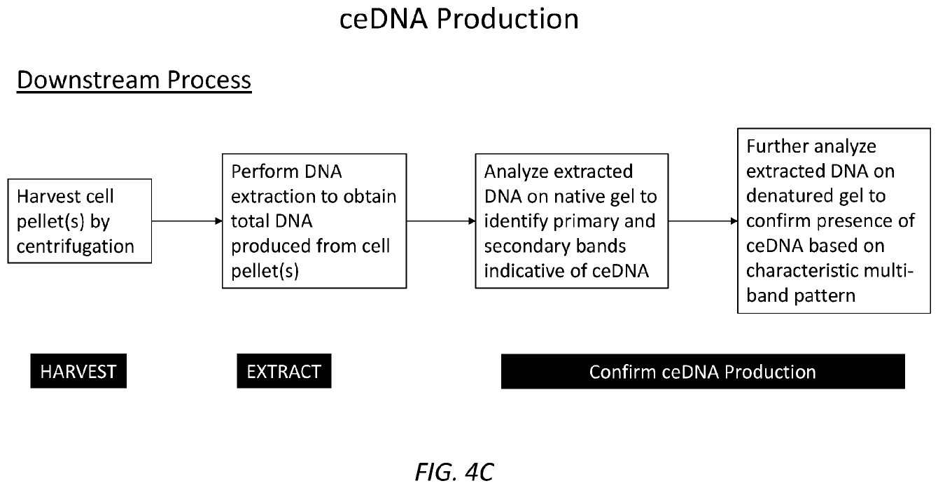

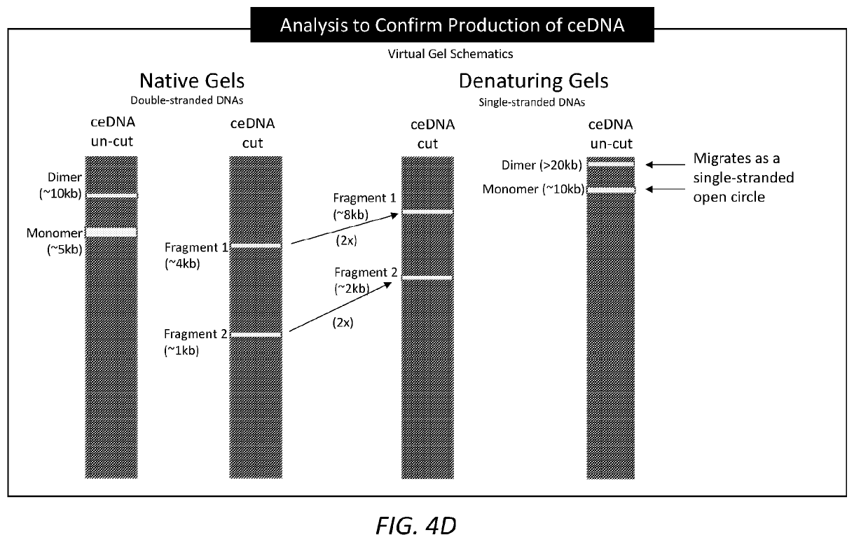

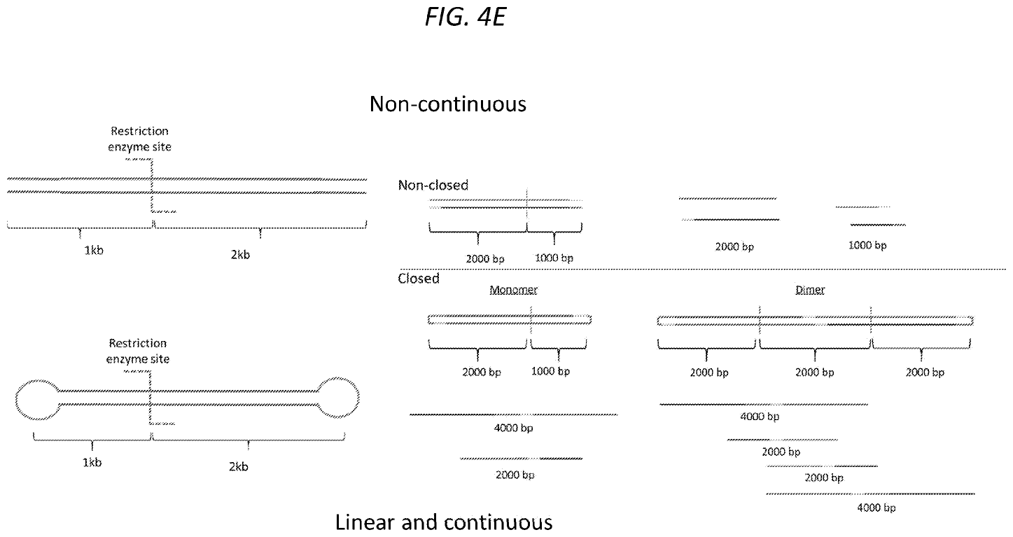

ceDNA vectors preferably have a linear and continuous structure rather than a non-continuous structure, as determined by restriction enzyme digestion assay (FIG. 4D). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. In some embodiments, ceDNA vectors can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

The time for harvesting and collecting DNA vectors described herein from the cells can be selected and optimized to achieve a high-yield production of the DNA vectors. For example, the harvest time can be selected in view of cell viability, cell morphology, cell growth, and the like. Usually, cells can be harvested after sufficient time after baculoviral infection to produce DNA-vectors but before a majority of the cells start to die because of the viral toxicity. The DNA-vectors can be isolated, for example, using plasmid purification kits such as Qiagen Endo-Free™ Plasmid kits. Other methods developed for plasmid isolation can also be adapted for DNA-vectors. Generally, any nucleic acid purification method known in the art can be adopted.

Promoters: Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 18 (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 19), a CAG promoter, a human alpha 1-antitypsin (HAAT) promoter (e.g., SEQ ID NO: 21), and the like. In embodiments, these promoters are altered at their downstream intron containing end to include one or more nuclease cleavage sites. In embodiments, the DNA containing the nuclease cleavage site(s) is foreign to the promoter DNA.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to the cell, tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., the gene editing molecules, donor sequence, therapeutic proteins etc.). For example, the vector may comprise a promoter that is operably linked to the nucleic acid sequence encoding a therapeutic protein. The promoter operably linked to the therapeutic protein coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metallothionein. The promoter may also be a tissue specific promoter, such as a liver specific promoter, such as human alpha 1-antitypsin (HAAT), natural or synthetic. In one embodiment, delivery to the liver can be achieved using endogenous ApoE specific targeting of the composition comprising a ceDNA vector to hepatocytes via the low density lipoprotein (LDL) receptor present on the surface of the hepatocyte.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers, (e.g. SEQ ID NO: 22 and SEQ ID NO: 23).

Non-limiting examples of suitable promoters for use in accordance with the present invention include the CAG promoter of, for example (SEQ ID NO: 3), the HAAT promoter (SEQ ID NO: 21), the human EF1-a promoter (SEQ ID NO: 6) or a fragment of the EF la promoter (SEQ ID NO: 15) and the rat EF1-a promoter (SEQ ID NO: 24).

Polyadenylation Sequences: A sequence encoding a polyadenylation sequence can be included in the ceDNA vector to stabilize the mRNA expressed from the ceDNA vector, and to aid in nuclear export and translation. In one embodiment, the ceDNA vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

In some embodiments, the ceDNA can be obtained from a vector polynucleotide that encodes a heterologous nucleic acid operatively positioned between two different inverted terminal repeat sequences (ITRs) (e.g. AAV ITRs), wherein at least one of the ITRs comprises a terminal resolution site and a replicative protein binding site (RPS), e.g. a Rep binding site (e.g. wt AAV ITR SEQ ID NO: 1 or SEQ ID NO: 2 for AAV2), and one of the ITRs comprises a deletion, insertion, and/or substitution with respect to the other ITR, e.g. functional ITR.

The host cells do not express viral capsid proteins and the polynucleotide vector template is devoid of any viral capsid coding sequences. In one embodiment, the polynucleotide vector template is devoid of AAV capsid genes but also of capsid genes of other viruses). In addition, in a particular embodiment, the nucleic acid molecule is also devoid of AAV Rep protein coding sequences. Accordingly, in a preferred embodiment, the nucleic acid molecule of the invention is devoid of both functional AAV cap and AAV rep genes.

In certain embodiments of the present invention, the ceDNA vector does not have a modified ITRs comprising the nucleotide sequence selected from the group consisting of SEQ ID NOs: 548, 549, 551, 552, 553, 553, 554, 555, 556 and 557.

In some embodiments, the ceDNA vector comprises a regulatory switch as disclosed herein (or in PCT application No. PCT/US18/49996, filed Sep. 7, 2018) and a modified ITR having the nucleotide sequence selected from the group consisting of: SEQ ID NOs: 548, 549, 551, 552, 553, 553, 554, 555, 556 and 557.

Without limitation, we state that the above reservation of a right of disclaimer applies at least to claims 1-33 of this application and paragraphs as set forth at [00293] and [00294].

Some embodiments of the various aspects disclosed herein can be defined by any one of the following paragraphs:
1. A liposomal ceDNA vector, comprising a liposome encapsulating a ceDNA vector, the ceDNA vector comprising:
   an expression cassette comprising a cis-regulatory element, wherein the cis-regulatory element is selected from the group consisting of a posttranscriptional regulatory element and a BGH poly-A signal;
   a wild-type ITR on the upstream (5′-end) of the expression cassette, wherein the wild-type ITR comprises a polynucleotide of SEQ ID NO: 51; and
   a modified ITR on the downstream (3′-end) of the expression cassette, wherein the modified ITR comprises a polynucleotide of SEQ ID NO:2,
      wherein said DNA vector has is devoid of a prokaryote-specific methylation, and is not encapsidated in an AAV capsid protein.
2. The DNA vector of paragraph 1, wherein the DNA vector has a linear and continuous structure.
3. The DNA vector of any of paragraphs 1-2, wherein the posttranscriptional regulatory element comprises a WHP posttranscriptional regulatory element (WPRE).
4. The DNA vector of any of paragraphs 1-3, wherein the expression cassette further comprises a cloning site.
5. The DNA vector of any of paragraphs 1-4, wherein the expression cassette comprises a promoter selected from the group consisting of CAG promoter, AAT promoter, LP1 promoter, and EF 1 a promoter.
6. The DNA vector of paragraph 1, wherein the expression cassette comprises polynucleotides of SEQ ID NO: 3, SEQ ID NO: 7, SEQ ID NO: 8 and SEQ ID NO: 9.
7. The DNA vector of any of paragraphs 1-6, wherein the expression cassette further comprises a cloning site and an exogenous sequence inserted into the cloning site.
8. The DNA vector of paragraph 7, wherein the exogenous sequence comprises at least 2000 nucleotides.
9. The DNA vector of paragraph 7, wherein the exogenous sequence encodes a protein.
10. The DNA vector of paragraph 7, wherein the exogenous sequence encodes a reporter protein.

Some embodiments of the various aspects disclosed herein can be defined by any one of the following paragraphs:
1. A lipid particle comprising an ionizable lipid and a non-viral capsid-free DNA vector with covalently-closed ends (ceDNA vector), wherein the ceDNA vector comprises at least one heterologous nucleotide sequence operably positioned between asymmetric inverted terminal repeat sequences (asymmetric ITRs), wherein at least one of the asymmetric ITRs comprises a functional terminal resolution site and a Rep binding site.
2. The lipid nanoparticle of paragraph 1, wherein the ceDNA vector when digested with a restriction enzyme having a single recognition site on the ceDNA vector and analyzed by both native and denaturing gel electrophoresis displays characteristic bands of linear and continuous DNA as compared to linear and non-continuous DNA controls.
3. The lipid nanoparticle of paragraph 1 or 2, wherein one or more of the asymmetric ITR sequences are from a virus selected from a parvovirus, a dependovirus, and an adeno-associated virus (AAV).
4. The lipid nanoparticle of paragraph 3, wherein the asymmetric ITRs are from different viral serotypes.
5. The lipid nanoparticle of of paragraph 4, wherein the one or more asymmetric ITRs are from an AAV serotype selected from AAV1, AAV2, AAV3, AAV4, AAV5, AAV6, AAV7, AAV8, AAV9, AAV10, AAV11, and AAV12.
6. The lipid nanoparticle of any one of paragraphs 1-3, wherein one or more of the asymmetric ITR sequences are synthetic.
7. The lipid nanoparticle of any one of paragraphs 1-6, wherein one or more of the ITRs is not a wild type ITR.
8. The lipid nanoparticle of any one of paragraphs 1-7, wherein one or more both of the asymmetric ITRs is modified by a deletion, insertion, and/or substitution in at least one of the ITR regions selected from A, A′, B, B′, C, C′, D, and D′.
9. The lipid nanoparticle of any one of paragraphs 1-8, wherein the ceDNA vector comprises at least two asymmetric ITRs selected from:
a. SEQ ID NO: 1 and SEQ ID NO:52; and
b. SEQ ID NO: 2 and SEQ ID NO: 51.
10. The lipid nanoparticle of any one of paragraphs 1-9, wherein the ceDNA vector is obtained from a process comprising the steps of:
a. incubating a population of insect cells harboring a ceDNA expression construct in the presence of at least one Rep protein, wherein the ceDNA expression construct encodes the ceDNA vector, under conditions effective and for a time sufficient to induce production of the ceDNA vector within the insect cells; and
b. isolating the ceDNA vector from the insect cells.
11. The lipid nanoparticle of paragraph 10, wherein the ceDNA expression construct is selected from a ceDNA plasmid, a ceDNA bacmid, and a ceDNA baculovirus.
12. The lipid nanoparticle of paragraph 10 or paragraph 11, wherein the insect cell expresses at least one Rep protein.
13. The lipid nanoparticle of paragraph 10, wherein at least one Rep protein is from a virus selected from a parvovirus, a dependovirus, and an adeno-associated virus (AAV)
14. The lipid nanoparticle of paragraph 13, wherein at least one Rep protein is from an AAV serotype selected from AAV1, AAV2, AAV3, AAV4, AAV5, AAV6, AAV7, AAV8, AAV9, AAV10, AAV11, and AAV12.
15. The lipid particle of any one of paragraphs 1-15, wherein the DNA vector is obtained from a vector polynucleotide, wherein the vector polynucleotide encodes a heterologous nucleic acid operatively positioned between two inverted terminal repeat sequences (ITRs), wherein the two ITS are different from each other (asymmetric), and at least one of the ITRs is a functional ITR comprising a functional terminal resolution site and a Rep binding site, and one of the ITRs comprises a deletion, insertion, and/or substitution relative to the functional ITR; the presence of Rep protein inducing replication of the vector polynucleotide and production of the DNA vector in an insect cell, the DNA vector being obtainable from a process comprising the steps of:
a. incubating a population of insect cells harboring the vector polynucleotide, which is devoid of viral capsid coding sequences, in the presence of Rep protein under conditions effective and for time sufficient to induce production of the capsid-free, non-viral DNA vector within the insect cells, wherein the insect cells do not comprise production of capsid-free, non-viral DNA within the insect cells in the absence of the vector; and
b. harvesting and isolating the capsid-free, non-viral DNA from the insect cells.
16. The lipid particle of any one of paragraphs 10-15, wherein the presence of the capsid-free, non-viral DNA isolated from the insect cells can be confirmed.
17. The lipid particle of paragraph 16, wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.
18. The lipid particle of any one of paragraphs 1-17, wherein the DNA vector is obtained from a vector polynucleotide, wherein the vector polynucleotide encodes a heterologous nucleic acid operatively positioned between a first and a second AAV2 inverted terminal repeat DNA polynucleotide sequence (ITRs), with at least one of the ITRs having at least one polynucleotide deletion, insertion, and/or substitution with respect to the corresponding AAV2 wild type ITR of SEQ ID NO:1 or SEQ ID NO:51 to induce replication of the DNA vector in an insect cell in the presence of Rep protein, the DNA vector being obtainable from a process comprising the steps of: a. incubating a population of insect cells harboring the vector polynucleotide, which is devoid of viral capsid coding sequences, in the presence of Rep protein, under conditions effective and for a time sufficient to induce production of the capsid-free, non-viral DNA within the insect cells, wherein the insect cells do not comprise viral capsid coding sequences; and b. harvesting and isolating the capsid-free, non-viral DNA from the insect cells.
19. The lipid particle of paragraph 18, wherein the presence of the capsid-free, non-viral DNA isolated from the insect cells can be confirmed.
20. The lipid particle of paragraph 19, wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.
21. The lipid particle of any one of paragraphs 1-20, wherein the lipid particle further comprises one or more of a non-cationic lipid; a PEG conjugated lipid; and a sterol.
22. The lipid particle of any one of paragraphs 1-21, wherein the ionizable lipid is a lipid described in Table 1.
23. The lipid particle of any one of paragraphs 1-22, wherein the lipid particle further comprises a non-cationic lipid, wherein the non-ionic lipid is selected from the group consisting of di stearoyl-sn-glycero-phosphoethanol amine, di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-m al eimi d omethyl)-cy cl ohexane-l-carb oxyl ate (DOPE-mal), dipalmitoyl phosphatidyl ethanolamine (DPPE), dimyristoylphosphoethanolamine (DMPE), distearoyl-phosphatidyl-ethanolamine (D SPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-ol eoyl-pho sphati dy ethanol amine (S OPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), di stearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, and dilinoleoylphosphatidylcholine.
24. The lipid particle of any one of paragraphs 1-23, wherein the lipid particle further comprises a conjugated lipid, wherein the conjugated lipid, wherein the conjugated-lipid is selected from the group consisting of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di (tetrade canoyl oxy)propyl-1-O-(w-methoxy (p oly ethoxy)ethyl) butanedioate (PEG-S-DMG)), PEG dialkoxypropylcarbam, and N-(carbonyl-methoxypolyethylene glycol 2000)-1,2-distearoyl-sn-glycero-3 -phosphoethanolamine sodium salt.
25. The lipid particle of any one of paragraphs 1-24, wherein the lipid particle further comprises cholesterol or a cholesterol derivative.
26. The lipid particle of any one of paragraphs 1-25, wherein the lipid particle comprises:
(i) an ionizable lipid;
(ii) a non-cationic lipid;
(iii) a conjugated lipid that inhibits aggregation of particles; and
(iv) a sterol.
27. The lipid particle of any one of paragraphs 1-26, wherein the lipid particle comprises:
(a) an ionizable lipid in an amount from about 20 mol % to about 90 mol % of the total lipid present in the particle;
(b) a non-cationic lipid in an amount from about 5 mol % to about 30 mol % of the total lipid present in the particle;
(c) a conjugated lipid that inhibits aggregation of particles in an amount from about 0.5 mol % to about 20 mol % of the total lipid present in the particle; and
(d) a sterol in an amount from about 20 mol % to about 50 mol % of the total lipid present in the particle.
28. The lipid particle of any one of paragraphs 1-27, wherein total lipid to DNA vector (mass or weight) ratio is from about 10:1 to about 30:1.
29. A composition comprising a first lipid nanoparticle and an additional compound, wherein the first lipid nanoparticle comprises a first capsid free, non-viral vector, and is a lipid nanoparticle of any one of paragraphs 1-28.
30. The composition of paragraph 29, wherein said additional compound is encompassed in a second lipid nanoparticle, and wherein the first and second lipid nanoparticles are different.
31. The composition of paragraph 28 or 29, wherein said additional compound is encompassed in the first lipid nanoparticle.
32. The composition of any one of paragraphs 28-30, wherein said additional compound is a therapeutic agent.
33. The composition of paragraph 28, where said additional compound is a second capsid free, non-viral vector, wherein the first and second capsid free, non-viral vectors are different.

The following examples are provided by way of illustration not limitation.

### EXAMPLES

### Example 1

### Exemplary Method for Producing ceDNA Vectors

Construction of ceDNA Plasmids

Production of the ceDNA vectors using a polynucleotide construct template is described. For example, a polynucleotide construct template used for generating the ceDNA vectors of the present invention can be a ceDNA-plasmid, a ceDNA-bacmid, and/or a ceDNA-baculovirus. Without being limited to theory, in a permissive host cell, in the presence of e.g., Rep, the polynucleotide construct template having two ITRs and an expression construct, where at least one of the ITRs is modified, replicates to produce ceDNA vectors. ceDNA vector production undergoes two steps: first, excision (“rescue”) of template from the template backbone (e.g. ceDNA-plasmid, ceDNA-bacmid, ceDNA-baculovirus genome etc.) via Rep proteins, and second, Rep mediated replication of the excised ceDNA vector.

An exemplary method to produce ceDNA vectors is from a ceDNA-plasmid as described herein. Referring to FIGS. 1A and 1B, the polynucleotide construct template of each of the ceDNA-plasmids includes both a left ITR and a right mutated ITR with the following between the ITR sequences: (i) an enhancer/promoter; (ii) a cloning site for a transgene; (iii) a posttranscriptional response element (e.g. the woodchuck hepatitis virus posttranscriptional regulatory element (WPRE)); and (iv) a poly-adenylation signal (e.g. from bovine growth hormone gene (BGHpA). Unique restriction endonuclease recognition sites (R1-R6) (shown in FIGS. 1A and 1B) were also introduced between each component to facilitate the introduction of new genetic components into the specific sites in the construct. R3 (Pmel) GTTTAAAC (SEQ ID NO: 7) and R4 (PacI) TTAATTAA (SEQ ID NO: 542) enzyme sites are engineered into the cloning site to introduce an open reading frame of a transgene. These sequences were cloned into a pFastBac HT B plasmid obtained from ThermoFisher Scientific.

In brief, a series of ceDNA vectors were obtained from the ceDNA-plasmid constructs shown in Table 3, using the process shown in FIGS. 4A-4C. Table 5 indicates the number of the corresponding polynucleotide sequence for each component, including sequences active as replication protein site (RPS) (e.g. Rep binding site) on either end of a promoter operatively linked to a transgene. The numbers in Table 3 refer to SEQ ID NOs in this document, corresponding to the sequences of each component.

TABLE 3
Exemplary ceDNA constructs

| | ITR-L | Promoter | | ITR-R |
|---|---|---|---|---|
| Plasmid | (SEQ ID NO) | (SEQ ID NO) | Transgene | (SEQ ID NO) |
| Constuct-1 | 51 | 3 | Luciferase | 2 |
| Constuct-2 | 52 | 3 | Luciferase | 1 |
| Constuct-3 | 51 | 4 w/SV40 intr | Luciferase | 2 |
| Constuct-4 | 52 | 4 w/SV40 intr | Luciferase | 1 |
| Constuct-5 | 51 | 5 w/SV40 intr | Luciferase | 2 |
| Constuct-6 | 52 | 5 w/SV40 intr | Luciferase | 1 |
| Constuct-7 | 51 | 6 | Luciferase | 2 |
| Constuct-8 | 52 | 6 | Luciferase | 1 |

In some embodiments, a construct to make ceDNA vectors comprises a promoter which is a regulatory switch, e.g., an inducible promoter. Other constructs were used to make ceDNA vectors which comprise a MND or HLCR promoter operatively linked to a luciferase transgene.

Production of ceDNA-Bacmids:

With reference to FIG. 4A, DH10Bac competent cells (MAX EFFICIENCY® DH10Bac™ Competent Cells, Thermo Fisher) were transformed with either test or control plasmids following a protocol according to the manufacturers instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the □-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the PO virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25 ° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 p.m filter.

The ceDNA-baculovirus comprising the test contructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

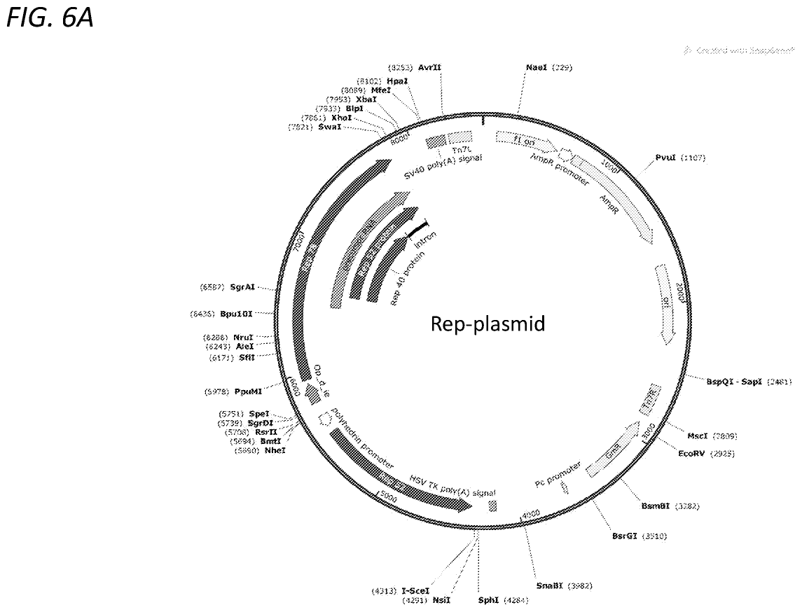

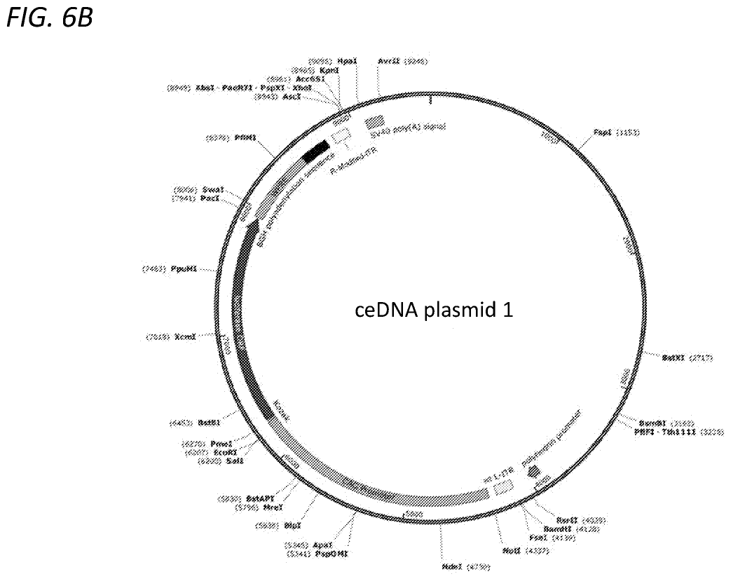

With referece to FIG. 4A, a “Rep-plasmid” according to FIG. 6A was produced in a pFASTBAC™-Dual expression vector (ThermoFisher) comprising both the Rep78 (SEQ ID NO: 13) or Rep68(SEQ ID NO: 12) and Rep52 (SEQ ID NO: 14).

The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZAM15 marker provides a-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into 519 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naive Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm. Yields of various ceDNA vectors determined based on UV absorbance are provided below in Table 4.

TABLE 4
Yield of ceDNA vectors from exemplary constructs.

| | | | | Estimated |
|---|---|---|---|---|
| | Culture | Culture Parameters | Yield | Yield |
| Construct | Volume | (Diameter in micrometers) | (mg/L) | (pg/cell) |
| construct-1 | 2x1L | Total: 6.02 × 10e6 | 15.8 | 5.23 |
| | | Viability: 53.3% | | |
| | | Diameter: 18.4 | | |

The ceDNA vectors can be assessed by identified by agarose gel electrophoresis under native or denaturing conditions as illustrated in FIG. 4D, where ceDNA vectors generate multiple bands on native gels, e.g. see FIG. 4D. Each band can represent vectors having a different conformation, e.g., monomeric, dimeric, etc. The presence of a single band under denaturing conditions and dual bands (corresponding to monomeric and dimeric forms) under nondenaturing conditions is characteristic of the presence of the ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIG. 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

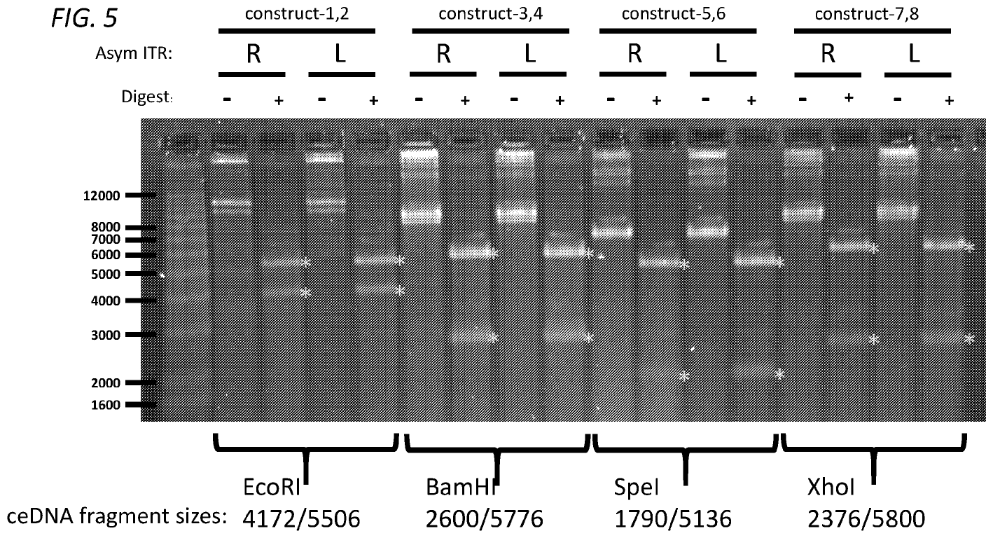

FIG. 5 provides an exemplary picture of a denaturing gel with ceDNA vectors as follows: construct-1, construct -2, construct-3, construct-4, construct-5, construct-6, construct-7 and construct-8 (all described in Table 3 above), with (+) or without (−) digestion by the endonuclease. Each ceDNA vector from constructs-1 to construct-8 produced two bands (*) after the endonuclease reaction. Their two band sizes determined based on the size marker are provided on the bottom of the picture. The band sizes confirm that each of the ceDNA vectors produced from plasmids comprising construct-1 to construct-8 has a continuous structure.

As used herein, the phrase “Assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately 1/3× and 2/3× of the DNA vector length. This resolves the bands on both native and denaturing gels. Before denaturation, it is important to remove the buffer from the sample. The Qiagen PCR clean-up kit or desalting “spin columns,” e.g. GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200mM NaOH to ensure that the NaOH concentration is uniform in the gel and gel box, and running the gel in the presence of lx denaturing solution (50 mM NaOH, 1 mM EDTA). One of ordinary skill in the art will appreciate what voltage to use to run the electrophoresis based on size and desired timing of results. After electrophoresis, the gels are drained and neutralized in lx TBE or TAE and transferred to distilled water or 1× TBE/TAE with 1× SYBR Gold. Bands can then be visualized with e.g. Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and nonlimiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 2

### ceDNA Vectors Express Luciferase Transgene In Vitro

Constructs were generated by introducing an open reading frame encoding the Luciferase reporter gene into the cloning site of ceDNA-plasmid constructs: construct-1, construct -3, construct -5, and construct -7. The ceDNA-plasmids (see above in Table 3) including the Luciferase coding sequence are named plasmid construct 1-Luc, c plasmid construct-3-Luc, plasmid construct -5-Luc, and plasmid construct 7-Luc, respectively.

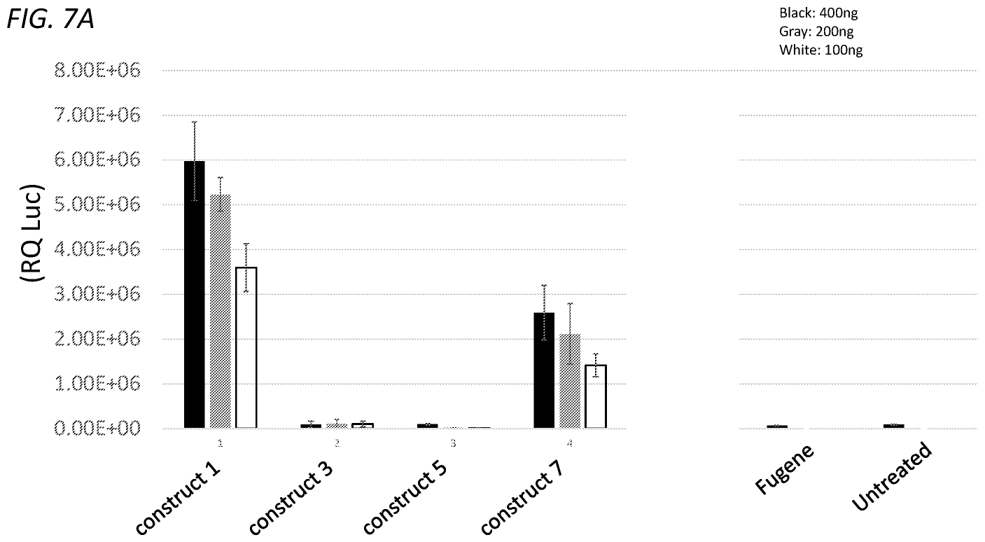

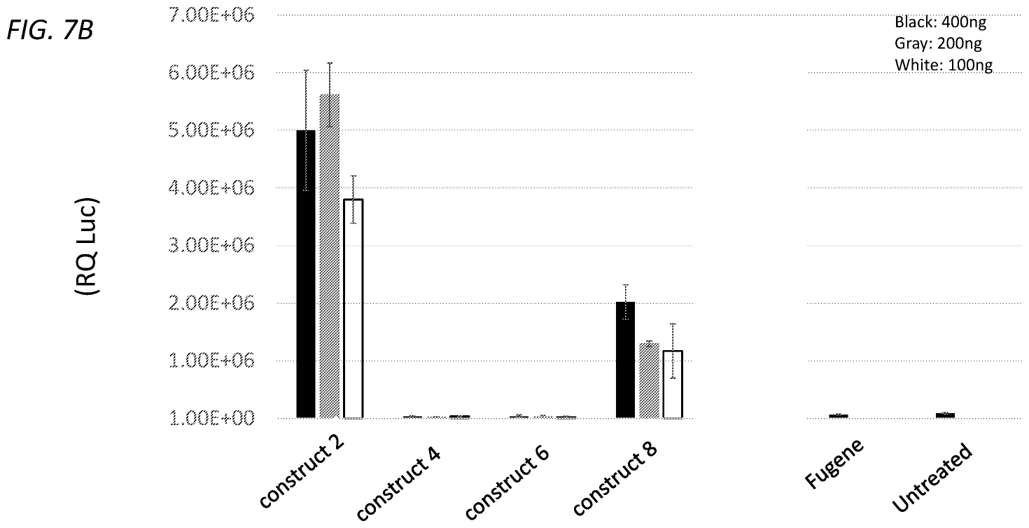

HEK293 cells were cultured and transfected with 100 ng, 200 ng, or 400 ng of plasmid constructs 1, 3, 5 and 7, using EUGENE® (Promega Corp.) as a transfection agent. Expression of Luciferase from each of the plasmids was determined based on Luciferase activity in each cell culture and the results are provided in FIG. 7A. Luciferase activity was not detected from the untreated control cells (“Untreated”) or cells treated with Fugene alone (“Fugene”), confirming that the Luciferase activity resulted from gene expression from the plasmids. As illustrated in FIGS. 7A and 7BB, robust expression of Luciferase was detected from constructs 1 and 7. The expression from construct-7 expressed Luciferase with a dose-dependent increase of Luciferase activity being detected.

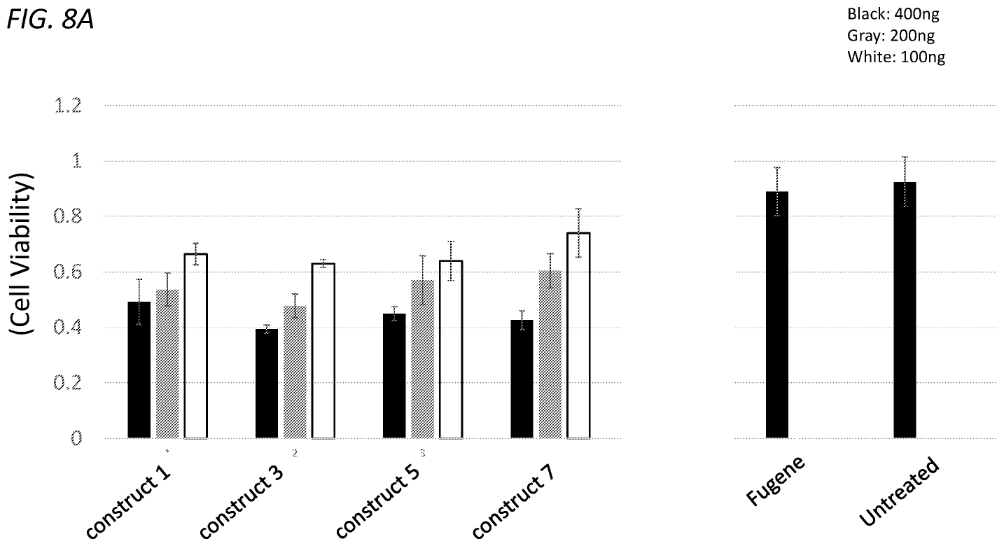

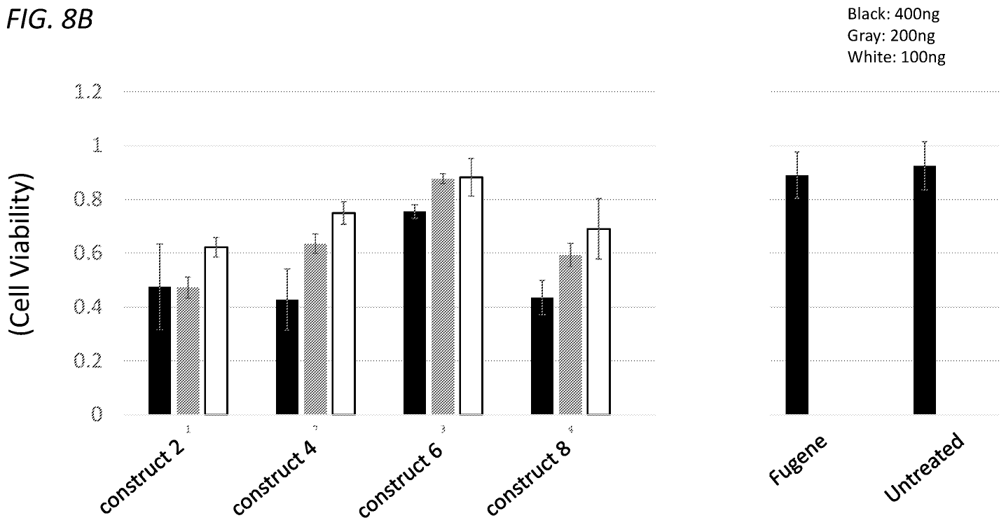

Growth and viability of cells transfected with each of the plasmids were also determined and presented in FIGS. 8A and 8B. Cell growth and viability of transfected cells were not significantly different between different groups of cells treated with different constructs.

Accordingly, Luciferase activity measured in each group and normalized based on cell growth and viability was not different from Luciferase activity without the normalization. ceDNA-plasmid with construct 1-Luc showed the most robust expression of Luciferase with or without normalization.

Thus, the data presented in FIGS. 7A, 7B, 8A and 8B demonstrate that construct 1, comprising from 5′ to 3′-WT-ITR (SEQ ID NO: 51), CAG promoter (SEQ ID NO:3), R3/R4 cloning site (SEQ ID NO:7), WPRE (SEQ ID NO: 8), BGHpA (SEQ ID NO:9) and a modified ITR (SEQ ID NO:2), is effective in producing a ceDNA vector that can express a protein of a transgene within the ceDNA vector.

### Example 3

### The lipid (6Z,9Z,28Z,31Z)-heptatriaconta-6,9,28,31-tetraen-19-yl-4-(dimethylamino) butanoate (DLin-MC3-DMA)

Lipid particles comprising ceDNA can be prepared or formulated in combination with the lipid (6Z,9Z,28Z,31Z)-heptatriaconta-6,9,28,31-tetraen-19-yl-4-(dimethylamino)-butanoate (DLin-MC3-DMA), also referred to as “MC3” herein, having the structure:

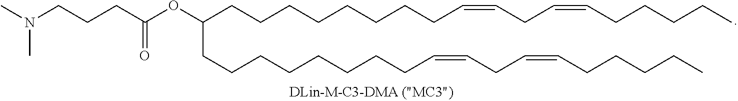 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety. It is synthesized as follows:

Synthesis of methanesulfonic acid octadeca-9,12-dienyl ester: To a solution of the alcohol 1 (26.6 g, 100 mmol) in dichloromethane (100 mL), triethylamine (13.13 g, 130 mmol) is added and this solution is cooled in an ice-bath. To this cold solution, a solution of mesyl chloride (12.6 g, 110 mmol) in dichloromethane (60 mL) is added dropwise and after the completion of the addition, the reaction mixture is allowed to warm to ambient temperature and stirred overnight. The TLC of the reaction mixture shows the completion of the reaction. The reaction mixture is diluted with dichloromethane (200 mL), washed with water (200 mL), satd. NaHCO₃ (200 mL), brine (100 mL) and dried (NaSO₄). The organic layer is concentrated to get the crude product which was purified by column chromatography (silica gel) using 0-10% Et₂O in hexanes. The pure product fractions are combined and concentrated to obtain the pure product methanesulfonic acid octadeca-9,12-dienyl ester as colorless oil. ¹H NMR (CDCl₃, 400 MHz) δ 5.42-5.21 (m, 4H), 4.20 (t, 2H), 3.06 (s, 3H), 2.79 (t, 2H), 2.19-2.00 (m, 4H), 1.90-1.70 (m, 2H), 1.06-1.18 (m, 18H), 0.88 (t, 3H). ¹³C NMR (CDCl₃) δ 130.76, 130.54, 128.6, 128.4, 70.67, 37.9, 32.05, 30.12, 29.87, 29.85, 29.68, 29.65, 29.53, 27.72, 27.71, 26.15, 25.94, 23.09, 14.60. MS. Molecular weight calculated for C₁₉H₃₆O₃S, 344.53.

Synthesis of 18-Bromo-octadeca-6,9-diene: The mesylate (methanesulfonic acid octadeca-9,12-dienyl ester, 13.44 g, 39 mmol) is dissolved in anhydrous ether (500 mL) and to it the MgBr.Et₂O complex (30.7 g, 118 mmol) is added under argon and the mixture is refluxed under argon for 26 h after which the TLC shows the completion of the reaction. The reaction mixture is diluted with ether (200 mL) and ice-cold water (200 mL) is added to this mixture and the layers are separated. The organic layer is washed with 1% aqueous K₂CO₃ (100 mL), brine (100 mL) and dried (anhyd. Na₂SO₄). Concentration of the organic layer provides the crude product which is further purified by column chromatography (silica gel) using 0-1% Et₂O in hexanes to isolate the product 18-Bromo-octadeca-6,9-diene as a colorless oil. ¹H NMR (CDCl₃, 400 MHz) δ 5.41-5.29 (m, 4H), 4.20 (d, 2H), 3.40 (t, J =7 Hz, 2H), 2.77 (t, J =6.6 Hz, 2H), 2.09-2.02 (m, 4H), 1.88-1.00 (m, 2H), 1.46-1.27 (m, 18H), 0.88 (t, J=3.9 Hz, 3H). ¹³C NMR (CDCl₃) δ 130.41, 130.25, 128.26, 128.12, 34.17, 33.05, 31.75, 29.82, 29.57, 29.54, 29.39, 28.95, 28.38, 27.42, 27.40, 25.84, 22.79, 14.28.

Synthesis of (6Z,9Z,28Z,31Z)-heptatriaconta-6,9,28,31-tetraen-19-ol (DLin-MeO H): To a flame dried 500 mL RB flask, freshly activated Mg turnings (2.4 g, 100 mmol) are added and the flask is equipped with a magnetic stir bar, an addition funnel and a reflux condenser. This set-up is degassed and flushed with argon and 10 mL of anhydrous ether is added to the flask via syringe. 18-Bromo-octadeca-6,9-diene (26.5 g, 80.47 mmol) is dissolved in anhydrous ether (50 mL) and added to the addition funnel. About 5 mL of this ether solution is added to the Mg turnings while stirring vigorously. An exothermic reaction is noticed (to confirm/accelerate the Grignard reagent formation, 5 mg of iodine is added and immediate decolorization is observed confirming the formation of the Grignard reagent) and the ether starts refluxing. The rest of the solution of the bromide is added dropwise while keeping the reaction under gentle reflux by cooling the flask in water. After the completion of the addition the reaction mixture is kept at 35 ° C. for 1 h and then cooled in ice bath. Ethyl formate (2.68 g, 36.2 mmol) is dissolved in anhydrous ether (40 mL) and transferred to the addition funnel and added dropwise to the reaction mixture with stirring. An exothermic reaction is observed and the reaction mixture started refluxing. After the initiation of the reaction the rest of the ethereal solution of formate is quickly added as a stream and the reaction mixture is stirred for a further period of 1 h at ambient temperature. The reaction is quenched by adding 10 mL of acetone dropwise followed by ice cold water (60 mL). The reaction mixture is treated with aq. H₂SO₄ (10% by volume, 300 mL) until the solution became homogeneous and the layers are separated. The aq. phase is extracted with ether (2×100 mL). The combined ether layers are dried (Na₂SO₄) and concentrated to get the crude product which is treated with 1 g of sodium in methanol (200 mL) at room temperature overnight. Upon completion of the reaction, most of the solvent is evaporated. The resulting mixture is poured into 150 mL of 5% hydrochloric acid solution. The aqueous phase is extracted with ether (2×150 mL). The combined ether extract is washed with water (2×100 mL), brine (100 mL), and dried over anhydrous sodium sulfate. Evaporation of the solvent gave the crude product which is purified by column (silica gel, 0-10% ether in hexanes) chromatography and the pure product fractions are evaporated to provide the product DLin-MeOH as a colorless oil. ¹H NMR (400 MHz,CDCl₃) δ 5.47-5.24 (m, 8H), 3.56 (dd, J=6.8, 4.2, 1H), 2.85-2.66 (m, 4H), 2.12-1.91 (m, 9H), 1.50-1.17 (m, 46H), 0.98-0.76 (m, 6H). ¹³C NMR (101 MHz, CDCl₃) δ 130.41, 130.37, 128.18, 128.15, 77.54, 77.22, 76.91, 72.25, 37.73, 31.75, 29.94, 29.89, 29.83, 29.73, 29.58, 29.53, 27.46, 27.43, 25.89, 25.86, 22.80, 14.30.

Synthesis of 6Z, 9Z, 28Z, 31Z)-heptatriaconta-6,9 , 28, 31-tetraen-19-yl-4-(dimethylamino) butanoate (MC3) : The DLin-MeOH (144 g, 272 mmol) is dissolved in 1 L of dichloromethane and to it the hydrochloride salt of dimethylaminobutyric acid 7 (55 g, 328 mmol) is added followed by diisopropylethylamine (70 mL) and DMAP (4 g). After stirring for 5 min. at ambient temperature, EDCI (80 g, 417 mmol) is added and the reaction mixture is stirred at room temperature overnight after which the TLC (silica gel, 5% MeOH in CH₂Cl₂) analysis shows complete disappearance of the starting alcohol. The reaction mixture is diluted with CH₂Cl₂ (500 mL) and washed with saturated NaHCO₃ (400 mL), water (400 mL) and brine (500 mL). The combined organic layers are dried over anhyd. Na2SO4 and solvents are removed in vacuo. The crude product thus obtained is purified by Flash column chromatography [2.5 Kg silica gel, Using the following eluents i) column packed with 6L of 0.1% NEt₃ in DCM; after loading ii) 4 L of 0.1% NEt₃ in DCM; iii) 16 L of 2% MeOH—98% of 0.1% NEt₃ in DCM; iv) 4 L of 2.5% MeOH—97.5% of 0.1% Neta in DCM; v) 12 L of 3% MeOH—97% of 0.1% NEt₃ in DCM] to isolate the pure product MC3 as a colorless oil. ¹H NMR (400 MHz, CDCl₃): δ 5.46-5.23 (m, 8H), 4.93-4.77 (m, 1H), 2.83-2.66 (m, 4H), 2.37-2.22 (m, 4H), 2.20 (s, 6H), 2.10-1.96 (m, 9H), 1.85-1.69 (m, 2H), 1.49 (d, J=5.4, 4H), 1.39-1.15 (m, 39H), 0.95-0.75 (m, 6H). ¹³C NMR (101 MHz, CDCl₃): δ 173.56, 130.38, 130.33, 128.17, 128.14, 77.54, 77.22, 76.90, 74.44, 59.17, 45.64, 34.36, 32.69, 31.73, 29.87, 29.76, 29.74, 29.70, 29.56, 29.50, 27.44, 27.41, 25.84, 25.55, 23.38, 22.78, 14.27. EI-MS (+ve): MW calc. for C₄₃H₇₉NO₂ (M+H)⁺: 642.6.

### Example 4

### The Lipid ATX-002

Lipid particles comprising ceDNA can be prepared or formulated in combination with the lipid ATX-002 having the structure:

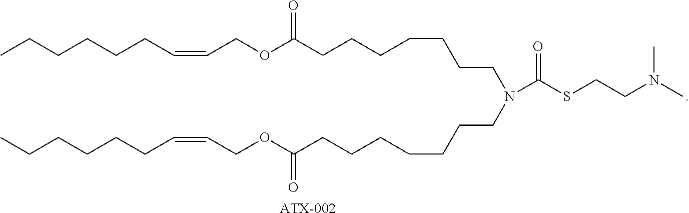

The lipid ATX-002 is described in WO2015/074085, content of which is incorporated herein by reference in its entirety. It is synthesized as follows:

Synthesis ofmethyl 8-bromooctanoate: Under N2 atmosphere, 8-bromooctanoic acid (60 gm, 1 eq.) is dissolved in 400 ml of dry methanol. Ten drops of concentrated H₂SO₄ is added dropwise and the reaction mixture was stirred under reflux for three hours.

The reaction is monitored by thin layer chromatography (TLC) until completed. Solvent is completely removed under vacuum. The reaction mixture is diluted with ethyl acetate and washed with water. The water layer is re-extracted with ethyl acetate. The total organic layer iswashed with a saturated NaHCO₃ solution. The organic layer is washed again with water and finally washed with brine. The product is dried over anhydrous Na₂SO₄ and concentrated.

Synthesis of dimethyl 8,8 ′-(benzanediyOdioctanoate: Dry K₂CO₃ (104.7 gm, 6 eq.) is taken and added to dry dimethylformamide under N₂. Benzyl amine (13.54 gm, 1 eq.) in dimethylformamide is slowly added. Methyl 8-bromooctanoate (60 gm, 2 eq.) dissolved in dimethylformamide is then added at room temperature. The reaction mixture is heated to 80° C. and the reaction is maintained for 36 hours with stirring.

The reaction is monitored by thin layer chromatography until completed. The reaction product is cooled to room temperature and water is added. The compound is extracted with ethyl acetate. The water layer is re-extracted with ethyl acetate. The total organic layer is washed with water and finally with brine solution. The product is dried over anhydrous Na₂SO₄ and concentrated.

The reaction product is purified by silica gel column chromatography in 3% methanol in chloroform. Using TLC system of 10% methanol in chloroform, the product migrates with a Rf: 0.8, visualizing by charring in ninhydrine. The compound is a light brown liquid. The structure is confirmed by ¹H-NMR.

Synthesis of dimethyl 8,8 ′-azanediyldioctanoate : Dimethyl 8,8′-(benzanediyl)dioctanoate (3.5 gm, 1 eq.) is transferred to hydrogenation glass vessel, and 90 ml of ethanol is added followed by 10% Pd/C (700mg). The reaction mixture is shaken in a Parr-shaker apparatus under 50 psi H₂ atmosphere pressure for two hours at room temperature. The reaction product is filtered through celite and washed with hot ethyl acetate. The filtrate is concentrated under vacuum.

Synthesis of dimethyl 8,8 ‘-((tertbutoxycarbonyl)azanedil) dioctanoate: Dimethyl 8,8’-azanediyl-dioctanoate (32 gm, 1 eq.) is transferred to dry DCM (700 ml) and dry Et₃N (9 gm, 4 eq.) to the reaction mass and cooled to 0° C. Boc anhydride (31.3 gm, 1.5 eq.) diluted in DCM is added drop wise to the above reaction. After the addition is completed, the reaction mixture is stirred at room temperature for three hours.

The reaction is quenched with water and the DCM layer is separated. The water phase is re-extracted with DCM and the combined DCM layers are washed with brine solution and dried with Na₂SO₄. After concentration, crude compound is collected. Crude reaction product is purified by column chromatography using 0-12% ethyl acetate in hexane. A single product migrates by thin layer chromatography in 20% ethyl acetate in hexane with an Rf of 0.5, charring with ninhydrine.

Synthesis of 8,8 4(tertbutoxycarbonyl)azanediyl)dioctanoic acid: Dimethyl 8,8′-((tertbutoxy-carbonyl)azanedil) dioctanoate (21 gm, 1 eq.) is transferred to dry THF (200 ml). A 6N aq. sodium hydroxide solution (175 ml) is added at room temperature. The reaction is maintained with stirring overnight at room temperature.

Reaction mass is evaporated under vacuum at 25° C. to remove THF. The reaction product is acidified with 5N HCl. Ethyl acetate is added to the aqueous layer. The separated organic layer is washed with water and the water layer was re-extracted with ethyl acetate. The combined organic layers are washed with brine solution and dried over anhydrous Na₂SO₄. Concentration of the solution gives the crude product.

Synthesis of di((Z)-non-2-en-1-yl) 8,8 ′((tertbutoxycarbonypazanediyl): 8,8′-((tertbutoxycarbonyl) azanediyl)dioctanoic acid (18 gm, 1 eq.) is dissolved in dry DCM (150 ml). HATU (26.15 gm, 2.1 eq.) is added to this solution. D-isopropyl ethyl amine (14.81 gm, 3.5eq.) is added slowly to the reaction mixture at room temperature. The internal temp rises to 40° C. and a pale yellow color solution is formed. DMAP (400 mg, 0.1 eq.) is added to the reaction mixture followed by cis-2-nonene-l-ol solution (9.31 gm, 2 eq.) in dry DCM. The reaction changes to brown color. The reaction is stirred for five hours at room temperature.

The reaction is checked by thin layer chromatography under completion. Water is added to the reaction product, which is extracted with DCM. The DCM layer is washed with water followed by brine solution. The organic layer is dried over anhydrous Na₂SO₄ and concentrated to obtain the crude compound.

Synthesis of ATX-002: Di((Z)-non-2-en-1-yl) 8,8′((tertbutoxycarbonyl) azanediyl) dioctanoate (13.85 mmol, 9 grams) is dissolved in dry DCM (150 ml). TFA is added at 0° C. to initiate a reaction. The reaction temperature is slowly allowed to warm to room temperature over for 30 minutes with stirring. Thin layer chromatography shows that the reaction is complete. The reaction product is concentrated under vacuum at 40° C. and the crude residue is diluted with DCM, and washed with a 10% NaHCO₃ solution. The aqueous layer is re-extracted with DCM, and the combined organic layers are washed with brine solution, dried over Na₂SO₄ and concentrated. The collected crude product is dissolved in dry DCM (85 ml) under nitrogen gas. Triphosgene are added and the reaction mixture is cooled to 0° C., and Et₃N is added drop wise. The reaction mixture is stirred overnight at room temperature. This layer chromatography shows that the reaction is complete. DCM solvent is removed from the reaction mass by distillation under N2. The reaction product is cooled to 0° C., diluted with DCM (50 ml), and 2-(dimethylamino)ethanethiol HCl (0.063 mol, 8.3 g) is added, followed by Et₃N (dry). The reaction mixture is then stirred overnight at room temperature. Thin layer chromatography shows that the reaction is complete. The reaction product is diluted with 0.3M HCl solution (75 ml), and the organic layer is separated. The aqueous layer is re-extracted with DCM, and the combined organic layers are washed with 10% K₂CO₃ aqueous solution (75 ml) and dried over anhydrous Na₂SO₄. Concentration of the solvent gives the crude product. The crude compound is purified by silica gel column (100-200 mesh) using 3% MeOH/DCM.

### Example 5

### (13Z,16Z)-N,N-dimethyl-3-nonyldocosa-13,16-dien-1-amine (Compound 32)

Lipid particles comprising ceDNA can be prepared or formulated in combination with the (13Z,16Z)-N,N-dimethyl-3-nonyldocosa-13,16-dien-1-amine (Compound 32) having the structure:

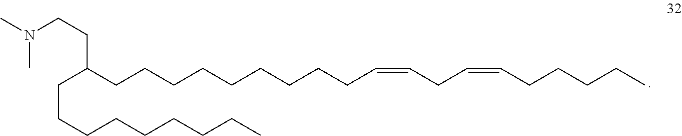 [SMILES: CCCCC/C=C\C/C=C\CCCCCCCCCC(CCCCCCCCC)CCN(C)C]

Compound 32 is described in WO2012/040184, content of which is incorporated herein by reference in its entirety. It is synthesized as follows:

### Synthesis of α, β-unsaturated amide (vii):

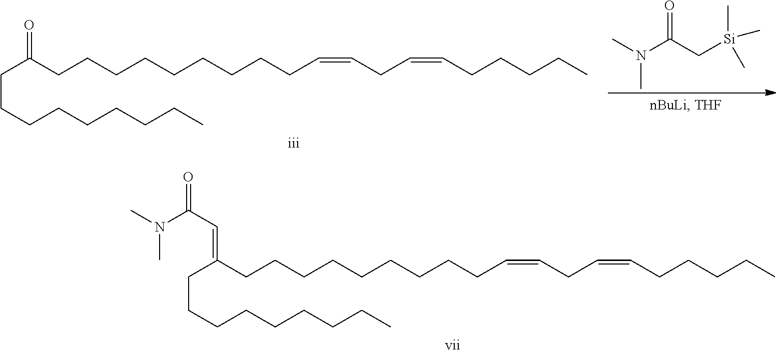

The silyl amide Peterson reagent (3.1 g, 16.7 mmol) is dissolved in THF(35 mL) and cooled to −63° C. To this solution is added nBuLi (16.7 mmo1, 6.7 mL of a 2.5M solution). The reaction is warmed to ambient temperature for 30 minutes. The ketone (iii) (5.0 g, 11.9 mmol) is dissolved in THF (25 mL) in a second flask. The ketone solution is transferred to the Peterson reagent over 30 minutes while maintaining the temperature between −60° C. and −40° C. The reaction is warmed to −40° C. for 1 hour, then warmed to 0° C. for 30 minutes. The reaction is quenched with sodium bicarbonate, diluted with additional water and partitioned between water/hexanes. The organics are washed with brine, dried over sodium sulfate, filtered and evaporated in vacuo. Purification by flash chromatography (0-40% MTBE/hexanes) gives the α,β-unsaturated amide (vii). ¹H NMR (400 MHz, CDCl₃) δ 5.75 (s, 1H), 5.36 (m, 4H), 3.01 (s, 3H), 2.99 (s, 3H), 2.78 (t, 2H), 2.28 (t, 2H), 2.05 (m, 6H), 1.35 (m, 34H), 0.89 (m, 6H).

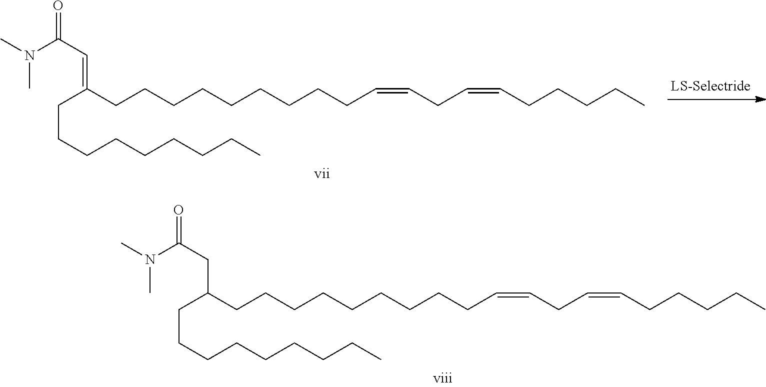

α,β-unsaturated amide (vii) (1 g, 2.1 mmol) and LS-Selectride (4.1 mmol, 4.1 mL of a 1M solution) are combined in a sealed tube and heated to 60° C. for 24 hours. The reaction is cooled to ambient temperature and partitioned between ammonium chloride solution and heptane. The organics are dried over sodium sulfate, filtered and evaporated in vacuo to give the amide (viii). This intermediate is carried directly into next reaction crude.

An alternate conjugate reduction of α,β-unsaturated amide (vii) involves the use of a copper hydride reduction.

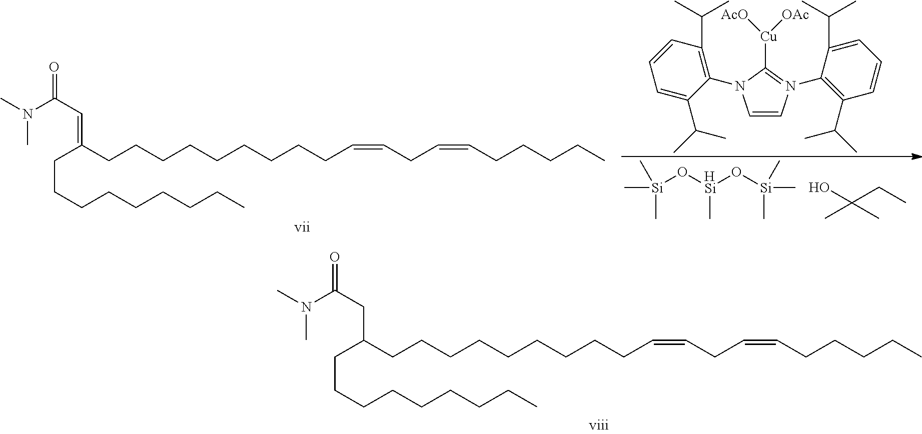

[In a 5 L RB, the Copper catalyst (9.77 g, 17.13 mmol) is dissolved in toluene (1713 ml) under nitrogen. To this is added the PMHS, from Aldrich (304 ml, 1371 mmol) in a single portion. The reaction is aged for 5 minutes. To the solutions is added the α,β-unsaturated amide (vii) (167.16 g, 343 mmol). To this mixture is then added the t-amyl alcohol (113 ml, 1028 mmol) over 3 h via syringe pump. After addition is complete, to the solution is added 1700 mL 20% NH4OH to reaction in small portions. Caution: there is a vigorous effervescence and foaming in the beginning of the quench and it must be closely monitored and the ammonium hydroxide added slowly in small portions. The reaction is partitioned between water and hexanes. The organics are filtered through celite and vaporated in vacuo. The resulting rubber solid material is pulverized using a mechanical stirrer in hexanes to give small particulates which are then filtered and washed with hexanes. The organics re then evaporated in vacuo and purified by flash chromatography (silica, 0-15% ethyl acetate/hexanes) to give desired amide (viii). LC/MS (M+H)=490.7.

Synthesis of Compound 32: To the solution of amide (viii) (2.85 g, 5.8 mmol) is added lithium aluminum hydride (8.7 mmol, 8.7 mL of a 1M solution). The reaction is stirred at ambient temperature for 10 minutes then quenched by slow addition of sodium sulfate decahydrate solution. The solids are filtered and washed with THF and the filtrate evaporated in vacuo. The crude mixture is purified by reverse phase preparative chromatography (C8 column) to provide (13Z,16Z)-N,N-dimethyl-3-nonyldocosa-13,16-dien-1-amine (Compound 32) as an oil. HRMS (M+H) calculated 476.5190. ¹H NMR (400 MHz, CDCl₃) δ 5.37 (m, 4H), 2.78 (t, 2H), 2.42 (m, 8H), 2.05 (q, 4H), 1.28 (m, 41H), 0.89 (m, 6H).

### Example 6

### Compounds 6 and 22

Lipid particles comprising ceDNA can be prepared or formulated in combination with the Compounds 6 or 22 having the structures:

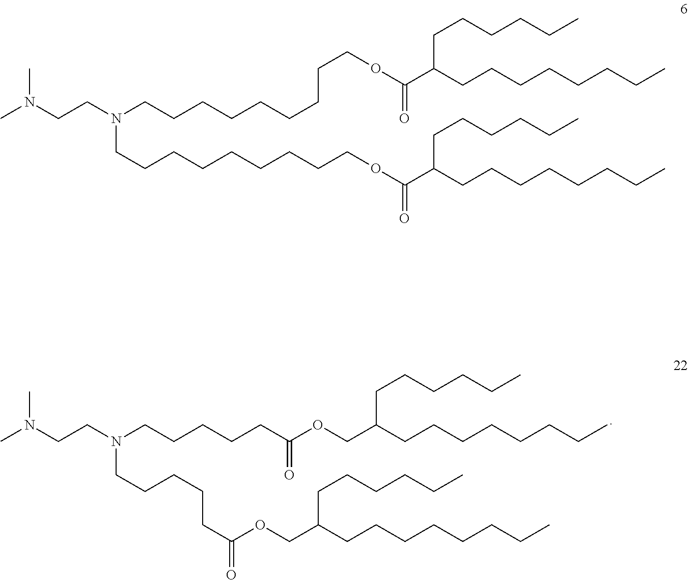

Compounds 6 and 22 are described in WO2015/199952, content of which is incorporated herein by reference in its entirety. Compounds 6 and 22 were synthesized as follows:

Synthesis of Compound 6: A solution of nonan-1,9-diol (12.6 g) in methylene chloride (80 mL) is treated with 2-hexyldecanoic acid (10.0 g), DCC (8.7 g), and DMAP (5.7 g). The solution is stirred for two hours. The reaction mixture is filtered and the solvent removed. The residue is dissolved in warmed hexane (250 mL) and allowed to crystallize. The solution is filtered and the solvent removed. The residue is dissolved in methylene chloride and washed with dilute hydrochloric acid. The organic fraction is dried over anhydrous magnesium sulfate, filtered and the solvent removed. The residue is passed down a silica gel column (75 g) using 0-12% ethyl acetate/hexane as the eluent, yielding 9-(2′-hexyldecanoyloxy)nonan-1-ol as an oil.

The product is dissolved in methylene chloride (60 mL) and treated with pyridinum chlorochromate (6.4 g) for four hours. Diethyl ether (200 mL) is added and the supernatant filtered through a silica gel bed. The solvent is removed from the filtrate and resultant oil passed down a silica gel (75 g) column using ethyl acetate/hexane (0-12%) gradient, yielding 9-(2′-ethylhexanoyloxy)nonanal as an oil.

A solution of the crude product (6.1 g), acetic acid (0.34 g) and 2-N,N-dimethylaminoethylamine (0.46 g) in methylene chloride (20 mL) is treated with sodium triacetoxyborohydride (2.9 g) for two hours. The solution is diluted with methylene chloride washed with aqueous sodium hydroxide, followed by water. The organic phase is dried over anhydrous magnesium sulfate, filtered and the solvent removed. The residue is passed down a silica gel (75 g) column using a methanol/methylene chloride (0-8%) gradient, followed by a second column (20 g) using a methylene chloride/acetic acid/methanol gradient. The purified fractions are dissolved in methylene chloride, washed with dilute aqueous sodium hydroxide solution, dried over anhydrous magnesium sulfate, filtered and the solvent removed, to yield the desired product as a colorless oil.

Synthesis of Compound 22: In step one, to a solution of 6-bromohexanoic acid (20 mmol, 3.901 g), 2-hexyl-1-decanol (1.8 eq, 36 mmol, 8.72 g) and 4-dimethylaminopyridine (DMAP 0.5 eq, 10 mmol, 1.22 g) in DCM (80 mL) is added DCC (1.1 eq, 22 mmol, 4.54 g). The resulting mixture is stirred at room temperature for 16 hours. The precipitate is discarded by filtration. The filtrate is concentrated. The residue is purified by column chromatography on silica gel eluted with gradient mixture of ethyl acetate in hexanes (0-2%). This gives the desired product as a colorless oil.

In step two, a mixture of the bromide from step one (1.34 eq, 7.88 g, 18.8 mmol), N,N-diisopropylethylamine (1.96 eq, 27.48 mmol, 4.78 mL) and N,N-dimethylethylenediamine (1 eq, 14.02 mmol, 1.236 g, 1.531 mL) in acetonitrile (70 mL) in 250 mL flask equipped with a condenser is heated at 79° C. (oil bath) for 16 hours. The reaction mixture is cooled to room temperature and concentrated. The residue is taken in a mixture of ethyl acetate and hexanes (1:9) and water. The phases are separated, washed with water (100 mL) and brine. It is then dried over sodium sulfate and concentrated (8.7 g oil). The crude (8.7 g oil) is purified by column chromatography on silica gel (0-3% MeOH in chloroform). The fractions containing the desired product are combined and concentrated. The residue is dissolved in 1 mL of hexane and filtered through a layer of silica gel (3-4 mm, washed with 8 mL of hexane). The filtrate is blown dry with a stream of Ar and dried well in vacuo overnight. The desired product is obtained as colorless oil. ¹H NMR (400 MHz, CDCl₃) δ 3.96 (d, 5.8 Hz, 4H), 2.55-2.50 (m, 2H), 2.43-2.39 (m, 4H), 3.37-3.32 (m, 2H), 2.30 (t, 7.5 Hz, 4H), 2.23 (s, 6H), 1.63 (quintet-like, 7.6 Hz, 6H), 1.48-1.40 (m, 4H), 1.34-1.20 (52H), 0.88 (t-like, 6.8 Hz, 12H).

### Example 7

### Preparation of Lipid Formulations

Lipid nanoparticles (LNP) can be prepared at a total lipid to ceDNA weight ratio of approximately 10:1 to 30:1. Briefly, an ionizable lipid (e.g., MC3, ATX-002, Compound 6, Compound 22, a compound of Formula (A′) or (A″), or a compound of Formula (B′), (B′) or (B″)), a non-cationic-lipid (e.g., distearoylphosphatidylcholine (DSPC)), a component to provide membrane integrity (such as a sterol, e.g., cholesterol) and a conjugated lipid molecule (such as a PEG-lipid, e.g., 1-(monomethoxy-polyethyleneglycol)-2,3-dimyristoylglycerol, with an average PEG molecular weight of 2000 (“PEG-DMG”)), are solubilized in alcohol (e.g., ethanol) at a molar ratio of 50:10:38.5:1.5. The ceDNA is diluted to a desired concentration in buffer solution. For example, the ceDNA can be diluted to a concentration of 0.1 mg/ml to 0.25 mg/ml in a buffer solution comprising sodium acetate, socium acetate and magnesium chloride, citrate, malic acid, or malic acid and sodium chloride. In one example, the ceDNA is diluted to 0.2 mg/mL in 10 to 50 mM citrate buffer, pH 4. The alcoholic lipid solution is mixed with ceDNA aqueous solution using, for example, syringe pumps or an impinging jet mixer, at a ratio of about 1:5 to 1:3 (vol/vol) with total flow rates above 10 ml/min. In one example, the alcoholic lipid solution is mixed with ceDNA aqueous at a ratio of about 1:3 (vol/vol) with a flow rate of 12 ml/min. The alcohol is removed and the buffer is replaced with PBS by dialysis. Alternatively, the buffer can be replaced with PBS using centrifugal tubes. Alcohol removal and simultaneous buffer exchange can be accomplished by, for example, dialysis or tangential flow filtration. The obtained lipid nanoparticles are filtered through a 0.2 pm pore sterile filter prior to further use.

### Example 8

### Analysis of Lipid Particle Formulations

Lipid nanoparticle particle size can be determined by quasi-elastic light scattering using a Malvern Zetasizer Nano ZS (Malvern, UK) and is approximately 55-95 nm diameter or approximately 70-90 nm diameter.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (20 1 0), both of which are incorporated by reference in their entirety). The preferred range of pKa is ˜5 to ˜7. The pKa of each cationic lipid is determined in lipid nanoparticles using an assay based on fluorescence of 2-(p-toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 μM stock solution in distilled water. Vesicles can be diluted to 24 μM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 μM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

Encapsulation of ceDNA in lipid particles can be determined by an Oligreen® assay. Oligreen® is an ultra-sensitive fluorescent nucleic acid stain for quantitating oligonucleotides and single-stranded DNA or RNA in solution (available from Invitrogen Corporation; Carlsbad, Calif.). As an alternative, PicoGreen° can be used. Briefly, encapsulation can be determined by performing a membrane-impermeable fluorescent dye exclusion assay, which uses a dye that has enhanced fluorescence when associated with nucleic acid. Encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated ceDNA, allowing it to interact with the membrane-impermeable dye. Encapsulation of ceDNA can be calculated as E=(I₀−I)/I₀, where/and Io refers to the fluorescence intensities before and after the addition of detergent.

Relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

### Example 9

### Evaluation of Exemplary ceDNA-LNPs

Lipid nanoparticles comprising exemplary ceDNAs were prepared at various N/P ratios (e.g., 3, 4, 5, and 6) using a lipid solution comprising MC3, DSPC, Cholesterol and DMG-PEG2000 (mol ratio 50:10:38.5:1.5). Aqueous solutions of a ceDNA vector in buffered solutions comprising salts such as sodium acetate, sodium acetate and magnesium chloride, citrate, malic acid, or malic acid and sodium chloride were prepared. The lipid solution and the ceDNA solution were mixed using an in-house procedure on a NanoAssembler at a total flow rate of 12 ml/min at a lipid to eDNA ratio of 1:3 (v/v).

### Characterization

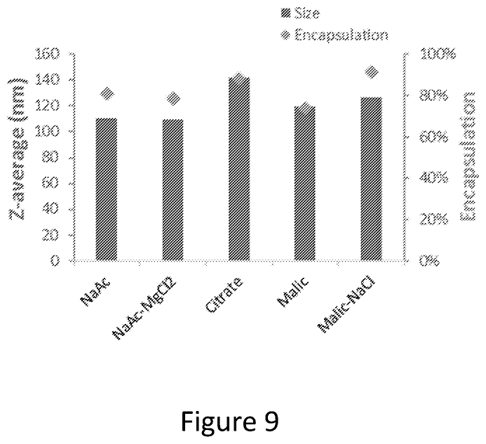

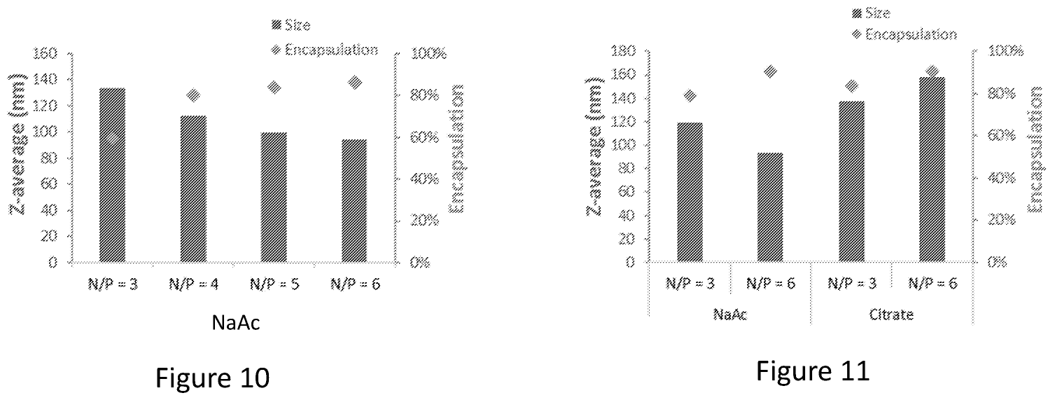

Lipid nanoparticle size and encapsulation of ceDNA into the lipid nanoparticles were determined. Particle size was determined by dynamic light scattering (ZEN3600, Malvern Instruments). Encapsulation efficiency was calculated by determining unencapsulated ceDNA content by measuring the fluorescence upon the addition of PicoGreen (Thermo Scientific) to free, the LNP slurry (C free) and comparing this value to the total ceDNA content that is obtained upon lysis of the LNPs by 1% Triton X-100 (Cₜₒₜₐₗ), where % encapsulation=(Cₜₒₜₐₗ−C_{free})/Cₜₒₜₐₗ×100. Results are shown in FIGS. 9-11.

### Endosomal Escape Assay

Effect of serum and BSA on encapsulation and release of ceDNA from LNPs were determined. Endosome mimicking anionic liposome was prepared by mixing DOPS:DOPC:DOPE (mol ratio 1:1:2) in chloroform, followed by solvent evaporation at vacuum. The dried lipid film was resuspended in DPBS with brief sonication, followed by filtration through 0.45 μm syringe filer to form anionic liposome.

LNP containing ceDNA and BSA or serum was mixed together at equal volumes. The mixture was incubated at 37° C. for 20 min. Subsequently, anionic liposome was added to LNP-BSA or serum mixture at desired anionic/cationic lipid mole ratio in DPBS at either pH 7.5 or 6.0. The resulting combination was then incubated at 37° C. for another 15 min. As a control, an equal volume of DPBS, in place of the anionic liposome, was added to the LNP-BSA or serum mixture. Free ceDNA with different treatments at pH 7.5 or pH 6.0 was calculated by determining unencapsulated ceDNA content by measuring the fluorescence upon free, the addition of PicoGreen (Thermo Scientific) to the LNP slurry (C_{free}) and comparing this value to the total ceDNA content that is obtained upon lysis of the LNPs by 1% Triton X-100 (Cₜₒₜₐₗ), where % free=C_{free}/Cₜₒₜₐₗ×100. The % ceDNA released after incubation with anionic liposome was calculated based on the equation below:

% ceDNA released=% free ceDNA_{mixed with anionic liposome}−% free ceDNA_{mixed with DPBS}

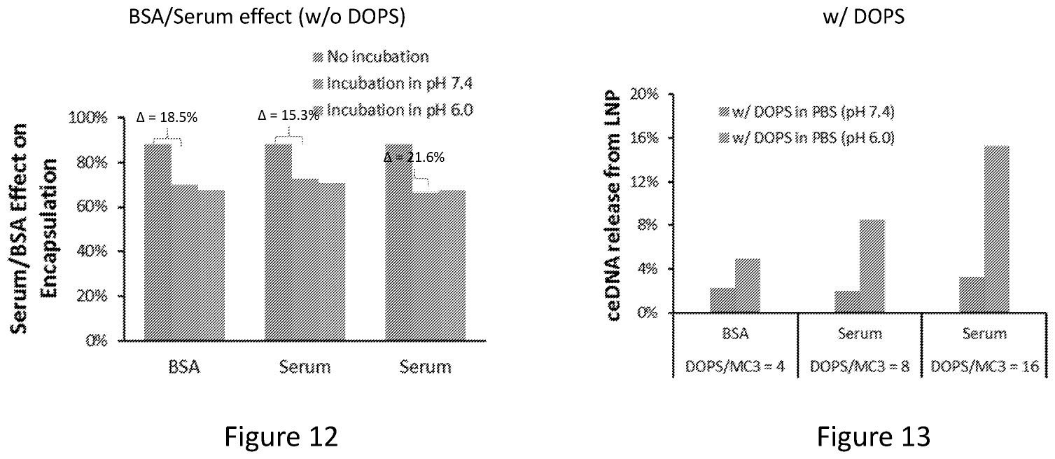

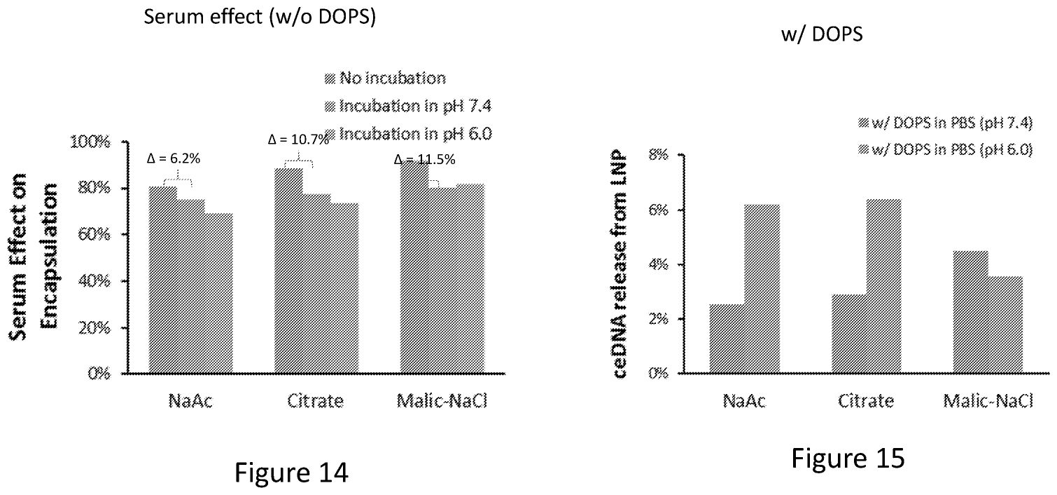

Results are shown in FIGS. 12-15 and summarized in Table 5.

TABLE 5
Release of ceDNA from exemplary LNPs are incubation with
DOPS liposome

| | MC3-ceDNA | | MC3-ceDNA | |
|---|---|---|---|---|
| DOPS⁻/ionizable lipid⁺ | 4 | | 16 | |
| Pre-incubation (1v:1v) | pH 7.4 | pH 6.0 | pH 7.4 | pH 6.0 |
| 50% CD1 serum | 1.9% | 8.5% | | |
| 100% CD1 serum | 2.6%* | 6.2%* | 3.3%* | 15.3%* |
| BSA | 2.3% | 4.9% | | |

*Serum containing 6 mM MgCl₂ and 200 nM actin

### ApoE Binding

Binding of the lipid nanoparticles to ApoE was determined. LNP (10 μg/mL) was incubated at 37° C. for 20 min with equal volume of recombinant ApoE3 (500 μg/mL) in 1× DPBS. After incubation, LNP samples were diluted 10-fold using lx DPBS and analyzed by heparin sepharose chromatography on AKTA pure 150 (GE Healthcare) according to the conditions below:

HiTrap chromatographic conditions

| Column | HiTrap Heparin Sepharose HP 1 mL | | |
|---|---|---|---|
| Equilibration buffer | 1x DPBS | | |
| Wash buffer | 1x DPBS | | |
| Elution buffer | 1M NaCl in 10 mM sodium phosphate buffer, pH 7.0 | | |
| Flow rate | 1 mL/min | | |
| Injection volume | 500 μL | | |
| Detection | 260 nm | | |
| | CV | A (%) | B (%) |
| Equilibration | 1 | 100 | 0 |
| Column wash | 4 | 100 | 0 |
| Elution (linear) | 10 | 0 | 100 |
| Equilibration | 3 | 100 | 0 |

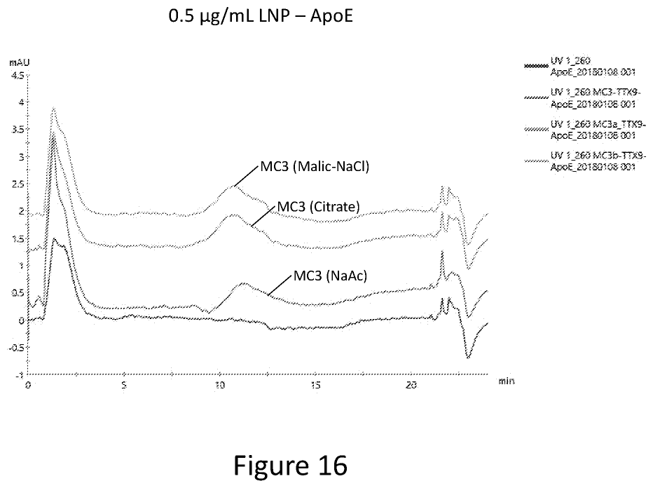

Results are shown in FIG. 16.

### HEK293 Expression

Expression of ceDNA encapsulated into the lipid nanoparticles was assayed as followa.

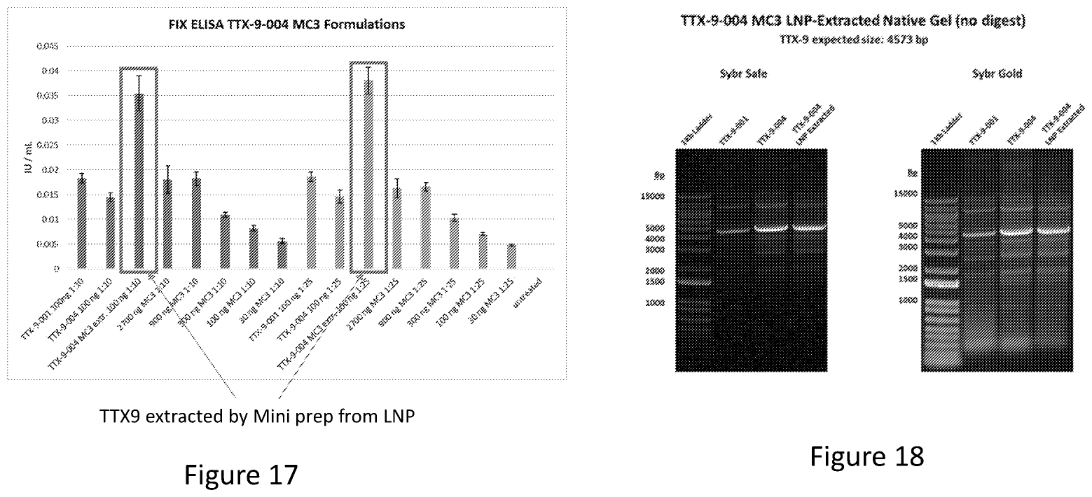

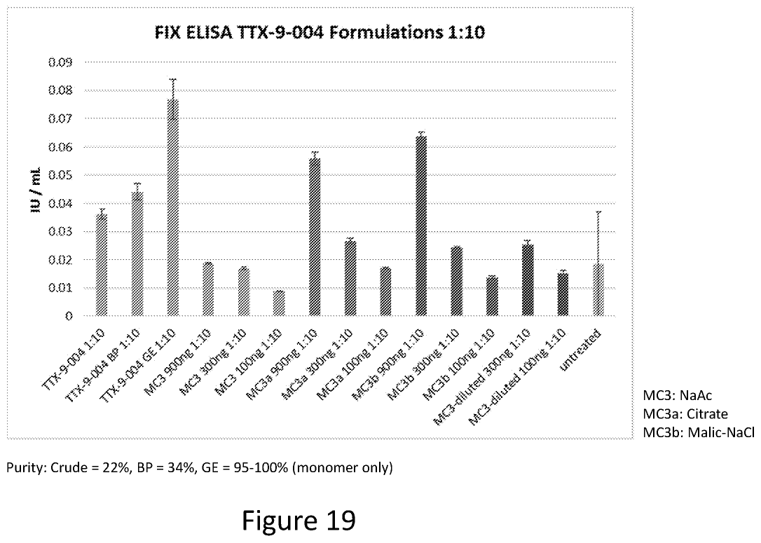

HEK293 cells were maintained at 37° C. with 5% CO₂ in DMEM +GlutaMAX™ culture medium (Thermo Scientific) supplemented with 10% Fetal Bovine Serum and 1% Penicillin-Streptomycin. Cells were plated in 96-well plates at a density of 30,000 cells/well the day before transfection. Lipofectamine™ 3000 (Thermo Scientific) transfection reagent was used for transfecting 100 ng/well of control ceDNA according to the manufacturer's protocol. The control ceDNA was diluted in Opti-MEM™ (Thermo Scientific) and P3000™ Reagent was added. Subsequently, Lipofectamine™ 3000 was diluted to a final concentration of 3% in Opti-MEM™. Diluted Lipofectamine™ 3000 was added to diluted ceDNA at a 1:1 ratio and incubated for 15 minutes at room temperature. Desired amount of ceDNA-lipid complex or LNP was then directly added to each well containing cells. The cells were incubated at 37° C. with 5% CO₂ for 72 hours. The expression levels of secreted Factor IX in HEK293-conditioned media were determined by VisuLize FIX Antigen ELISA Kit (Affinity Biologics) according to manufacturer's instructions. Results are shown in FIGS. 17-19.

### Example 10

### Compounds for Formulation A

Lipid nanoparticles (LNPs) comprising ceDNA can be prepared or formulated in combination with one or more compounds of Formula (I) or (II), preferably forming a liposome or lipid nanoparticle suitable for storage and therapeutic delivery of the ceDNA vector.

Compounds of Formula (I), useful in the preparation of lipid particle formulations, have the structure:

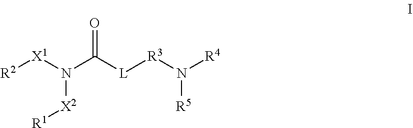

wherein
each of R¹, R², R⁴ and R⁵ is independently selected from the group consisting of hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, and cholesteryl;
R³ is selected from the group consisting of C₁-C₂₀ alkyl, substituted C₁-C₂₀ alkyl, C₂-C₂₀ alkenyl, substituted C₂-C₂₀ alkenyl, and C₂-C₂₀ alkynyl;
L is selected from the group consisting of S, O, C₁-C₂₀ alkenyl, substituted C₁-C₂₀ alkenyl;
X¹is absent,

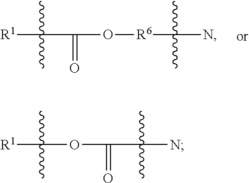

X² is absent,

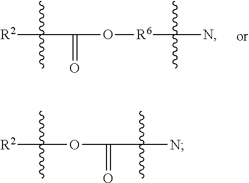

and each R⁶ is independently selected from the group consisting of C₁-C₂₀ alkyl, substituted
C₁-C₂₀ alkyl, C₂-C₂₀ alkenyl, substituted C₂-C₂₀ alkenyl, and C₂-C₂₀ alkynyl; or a salt thereof.

Compound of Formula (I), useful in the preparation of lipid particle formulations, have the structure:

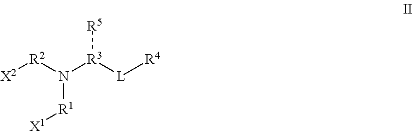

wherein
each of R¹, R², and R⁴ is independently selected from the group consisting of hydrogen, C₁-Cao alkyl, substituted C₁-C₂₀ alkyl, C₂-C₂₀ alkenyl, substituted C₂-C₂₀ alkenyl, C₂-C₂₀ alkynyl, and cholesteryl;
R³ is selected from the group consisting of C₁-C₂₀ alkyl, substituted C₁-C₂₀ alkyl, C₂-C₂₀ alkenyl, substituted C₂-C₂₀ alkenyl, and C₂-C₂₀ alkynyl;
R⁵ is absent or is selected from the group consisting of C₁-C₂₀ alkyl, substituted C₁-C₂₀ alkyl, C₂-C₂₀ alkenyl, substituted C₂-C₂₀ alkenyl, and C₂-C₂₀ alkynyl;
L is selected from the group consisting of S, O, C₁-C₂₀ alkenyl, substituted C₁-C₂₀ alkenyl;
X¹ is absent,

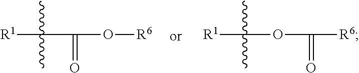

X² is absent,

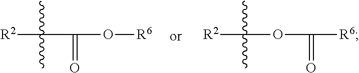

each R⁶ is independently selected from the group consisting of hydrogen, C₁-C₂₀ alkyl, substituted C₁-C₂₀ alkyl, C₂-C₂₀ alkenyl, substituted C₂-C₂₀ alkenyl, and C₂-C₂₀ alkynyl; and
[symbol] is a covalent bond to R⁵ when R⁵ is present or [symbol] is absent when R⁵ is absent; or a salt thereof.

In some exemplary compounds of Formula (I) or Formula (II), R¹ is a branched C₁₂-C₂₀ alkyl; R² is a linear C₅-C₁₀ alkyl or a branched C₁₂-C₂₀ alkyl; R⁴ and R³ are each independently a linear C₁-C₅ alkyl; each R⁶ is independently a linear or branched C₁-C₅ alkyl; and L is a linear C₁-C₃ alkyl.

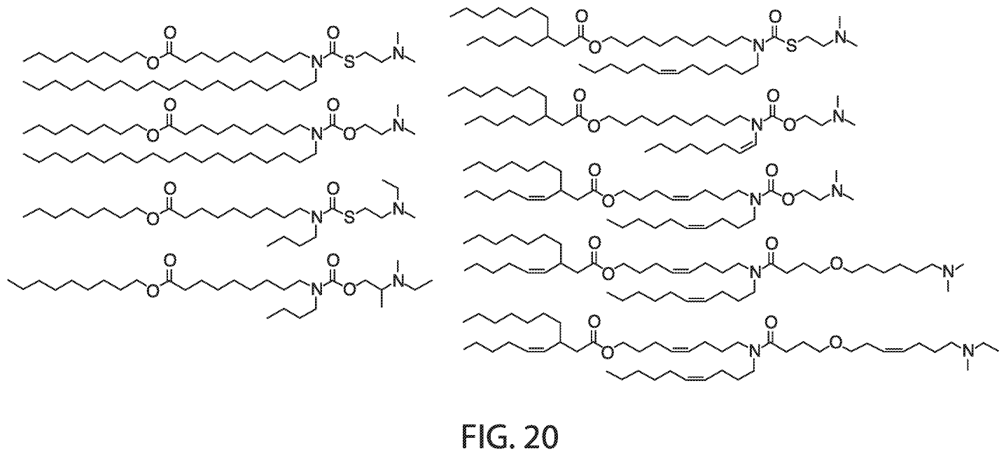

Exemplary compounds of Formula (I) or (II) can be one or more compounds selected from the compounds shown in FIG. 20.

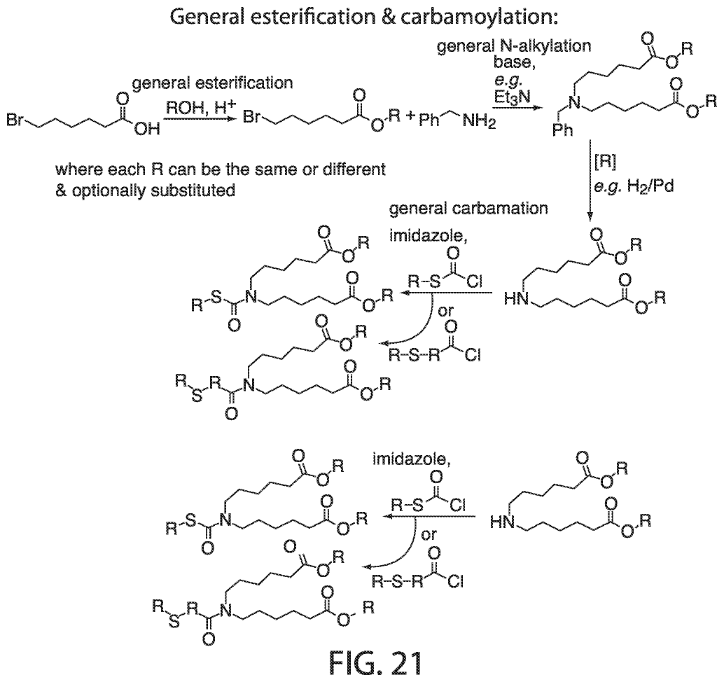

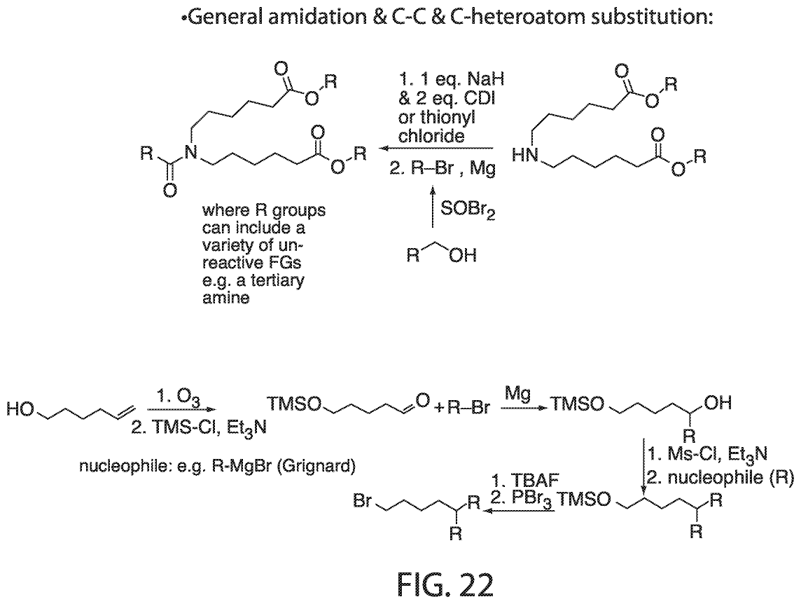

Generic syntheses of compounds of Formula (I) and (II) are shown in FIGS. 21 and 22, where each R value is independently selected.

In general, starting compounds disclosed above and other compounds can be purchased from commercial sources or prepared according to methods familiar to one of ordinary skill in the art. The skilled artisan will be able to construct even the most substituted scaffolds in the compounds described herein with conventional synthetic methods and through reference books and databases directed to chemical compounds and chemical reactions, as known to one of ordinary skill in the art. Suitable reference books and treatise that detail the synthesis of reactants useful in the preparation of compounds disclosed herein, or provide references to articles that describe the preparation of compounds disclosed herein, include for example, “Synthetic Organic Chemistry”, John Wiley and Sons, Inc. New York; S. R. Sandler et al, “Organic Functional Group Preparations,” rd. Ed., Academic Press, New York, 1983; H. 0. House, “Modem Synthetic Reactions,” 2nd Ed., W. A. Benjamin, Inc. Menlo Park, Calif., 1972; T. L. Glichrist, “Heterocyclic Chemistry,” 2nd Ed. John Wiley and Sons, New York, 1992; J. March, “Advanced Organic Chemistry: reactions, Mechanisms and Structure,” 5th Ed., Wiley Interscience, New York, 2001; Specific and analogous reactants may also be identified through the indices of known chemicals prepared by the Chemical Abstract Service of the American Chemical Society, which are available in most public and university libraries, as well as through online databases (the American Chemical Society, Washington, D.C. may be contacted for more details). Chemicals that are known but not commercially available in catalogs may be prepared by custom chemical synthesis companies, where many of the standard chemical supply companies further provide custom synthesis services.

### Example 11

### Compounds for Formulation B

Other lipid particles comprising ceDNA can be prepared or formulated in combination with one or more compounds of Formula (III), (IV) or (V) described in this example, preferably forming a liposome or lipid nanoparticle suitable for storage and therapeutic delivery of the ceDNA vector.

Compounds of Formula (III), useful in the preparation of lipid particle formulations, have the structure:

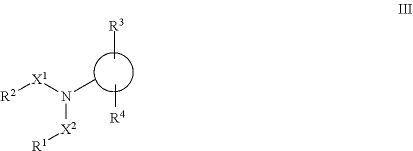

wherein
[symbol] represents a cyclic ring selected from the group consisting of C₃-C₁₀ cycloalkyl, C₃-C₁₀ cycloalkenyl, C₃-C₁₀ heterocycloalkyl, C₃-C₁₀ heterocycloalkenyl, C₆-C₁₀ aryl, and C₅-C₁₀ heteroaryl;
each of R¹, R², R³ and R⁴ is independently selected from the group consisting of hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, and cholesteryl; and

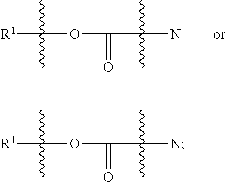

X¹ is absent, C₁-C₂₀ alkyl, substituted C₁-C₂₀ alkyl,

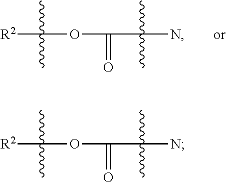

X² is absent, C₁-C₂₀ alkyl, substituted C₁-C₂₀ alkyl, or a salt thereof.

In some exemplary compounds of Formula (III), at least one of R¹, R², R³ and R⁴ is:

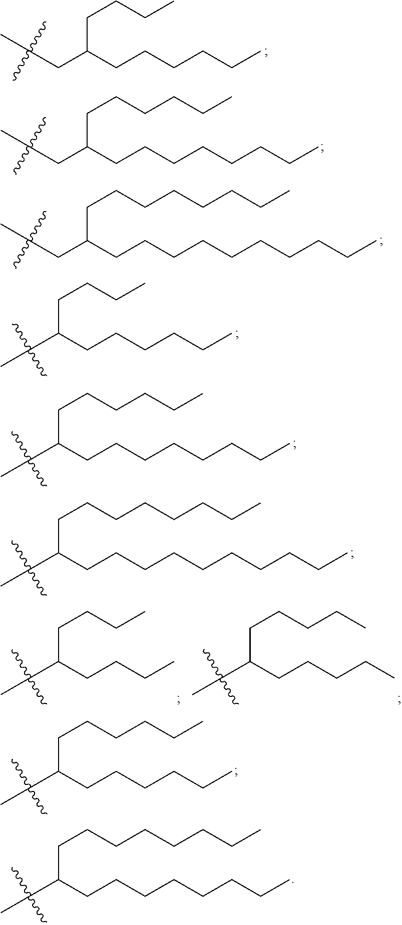

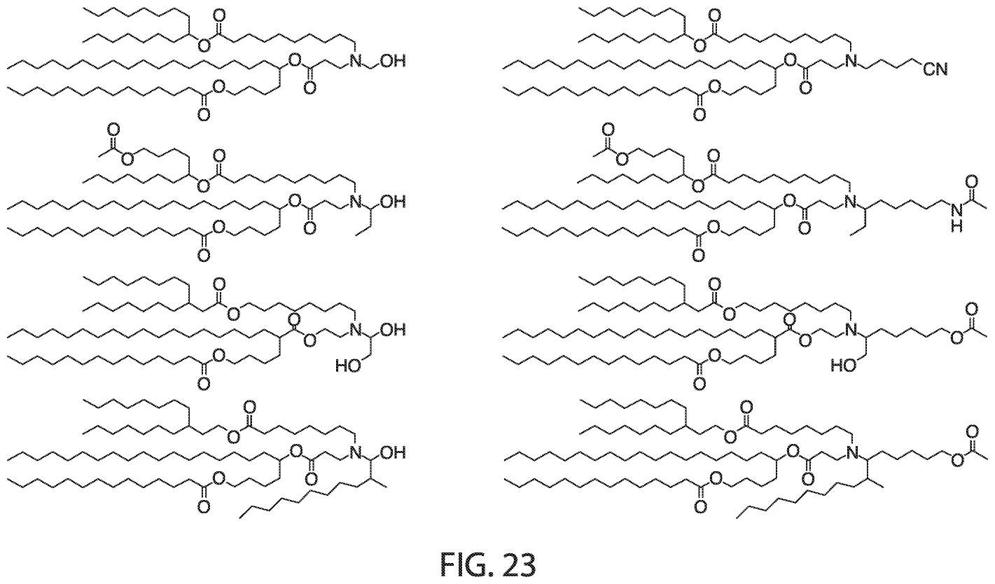

A compound of Formula (III) can be a compound selected from the compounds shown in FIG. 23.

Compounds of Formula (IV), useful in the preparation of lipid particle formulations, have the structure:

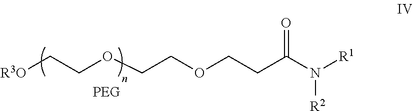

wherein:
n is from 0-150; and
each of R¹, R², and R³ is independently selected from the group consisting of hydrogen,

C₁-C₂₀ alkyl, substituted C₁-C₂₀ alkyl, C₂-C₂₀ alkenyl, substituted C₂-C₂₀ alkenyl, C₂-C₂₀ alkynyl, and cholesteryl;
or a salt thereof.

Compounds of Formula (V), useful in the preparation of lipid particle formulations, have the structure:

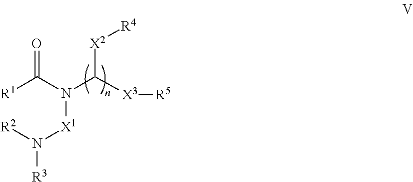

wherein
each of R¹, R², R³, R⁴ and R⁵ is independently selected from the group consisting of:

hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, and cholesteryl;
X¹ is absent, C₁-C₂₀ alkyl, substituted C₁-C₂₀ alkyl, or

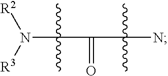

X² is absent, C₁-C₂₀ alkyl, substituted C₁-C₂₀ alkyl,

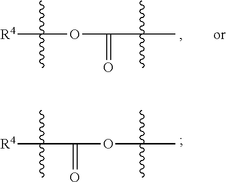

X³ is absent, C₁-C₂₀ alkyl, substituted C₁-C₂₀ alkyl,

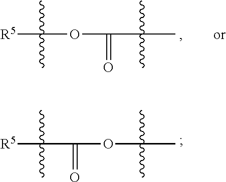

or a salt thereof.

Exemplary compounds of Formula (V) include, but are not limited to, the following:

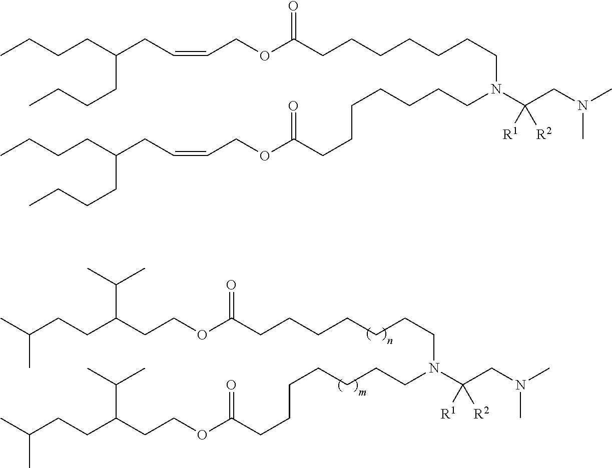

where n and m are each independently from 0-20; and
each R^{l} and R² is independently selected from the group consisting of: hydrogen, C₁-C₂₀ alkyl, substituted C₁-C₂₀ alkyl, C₂-C₂₀ alkenyl, substituted C₂-C₂₀ alkenyl, C₂-C₂₀ alkynyl, and cholesteryl;
or a salt thereof.

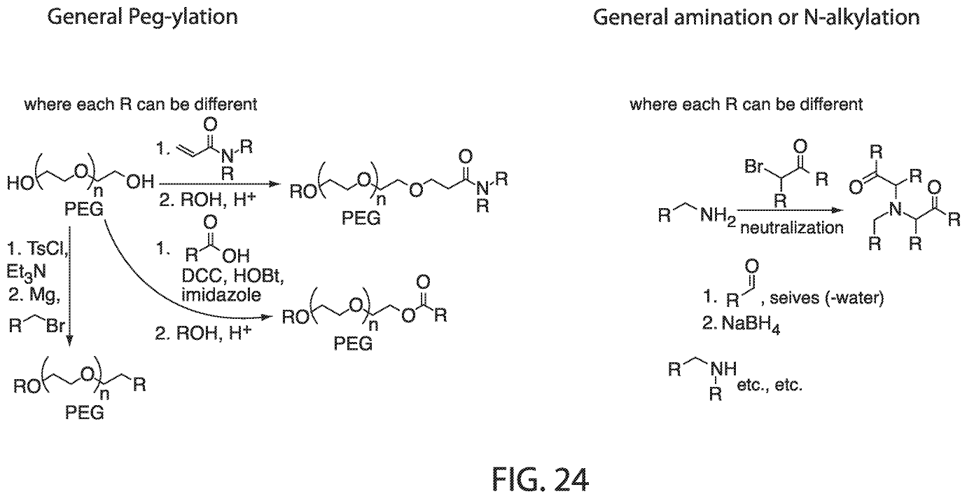

Generic syntheses of compounds of Formula (III), Formula (IV) and (V) is shown in FIG. 24, where each R value is independently selected.

It will be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley (herein incorporated by reference in its entirety). As one of skill in the art would appreciate, the protecting group may also be a polymer resin such as a Wang resin, Rink resin or a 2-chlorotrityl-chloride resin. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000), herein incorporated by reference in its entirety).

### Example 12

### Preparation of Lipid Formulations

Liposome Nanoparticles (LNP) can be prepared. Cationic lipid, DSPC, cholesterol and PEG-lipid can be solubilized in ethanol at a molar ratio of 50:10:38.5:1.5. Lipid nanoparticles (LNP) can be prepared at a total lipid to ceDNA weight ratio of approximately 10:1 to 30:1. Briefly, the ce DNA is diluted to 0.2 mg/mL in 10 to 50 mM citrate buffer, pH 4. Syringe pumps can be used to mix the ethanolic lipid solution with the ce DNA aqueous solution at a ratio of about 1:5 to 1:3 (vol/vol) with total flow rates above 15 ml/min. The ethanol can then be removed and the external buffer is replaced with PBS by dialysis. Finally, the lipid nanoparticles can be filtered through a 0.2 μm pore sterile filter. Lipid nanoparticle particle size is approximately 55-95 nm diameter, and in some instances is approximately 70-90 nm diameter as can be determined by quasi-elastic light scattering using a Malvern Zetasizer Nano ZS (Malvern, UK).

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (20 1 0), both of which are incorporated by reference in their entirety). The preferred range of pKa is ˜5 to ˜7. The pKa of each cationic lipid is determined in lipid nanoparticles using an assay based on fluorescence of 2-(p-toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 μM stock solution in distilled water. Vesicles can be diluted to 24 μM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 μM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

Lipid nanoparticles can also be formulated using the following molar 25 ratio: 50% Cationic lipid/10% distearoylphosphatidylcholine (DSPC)/38.5% Cholesterol/1.5% PEG lipid (“PEG-DMG”, i.e., (1-(monomethoxy-polyethyleneglycol)-2,3-dimyristoylglycerol, with an average PEG molecular weight of 2000). Relative activity can be determined by measuring luciferase expression in the liver 4 hours following administration via tail vein injection as described herein. The activity is compared at a dose of 0.3 and 1.0 mg ceDNA/kg and expressed as ng luciferase/g liver measured 4 hours after administration.

Therapeutic ceDNA construct compounds can be formulated using the following molar ratio: 50% cationic lipid/10% distearoylphosphatidylcholine (DSPC)/38.5% Cholesterol/1.5% PEG lipid (“PEG-DMA” 2-[2-(w-methoxy(polyethyleneglycol2000)ethoxy]-N ,N-ditetradecylacetamide). Relative activity can be determined by measuring luciferase expression in the liver 4 hours following administration via tail vein injection as described above. The activity can be compared at a dose of 0.3 and 1.0 mg mRNA or DNA/kg and expressed as ng luciferase/g liver measured 4 hours after administration, as described above.

### Example 13

### In Vivo Protein Expression of Luciferase Transgene from ceDNA Vectors

In vivo protein expression of a transgene from ceDNA vectors produced from the constructs 1-8 described above is assessed in mice. The ceDNA vector obtained from ceDNA-plasmid construct 1 (as described in Table 5 in Example 1) was tested and demonstrated sustained and durable luciferase transgene expression in a mouse model following hydrodynamic injection of a composition comprising the ceDNA vector comprised in lipid nanoparticles into the tail vein. Luciferase transgene expression was measured by IVIS imaging following intravenous administration into CD-1® IGS mice (Charles River Laboratories; WT mice).

The study assesses the biodistribution of hydrodynamic luciferase-expressing non-viral gene therapy vector by IVIS, following intravenous administration in CD-1® IGS mice. Vehicle is sterile PBS. In this study, two groups of five (5) CD-1 mice were administered either PBS or 0.35 mg/kg ceDNA encoding luciferase via hydrodynamic injection of 1.2 mL in the tail vein. Luciferase expression was assessed by IVIS imaging on Day 3, 4, 7, 14, 21, 28, 31, 35, and 42. Briefly, mice were injected intraperitoneally with 150 mg/kg of luciferin substrate and then whole body luminescence was assessed via IVIS imaging.

In vivo Luciferase expression: 5-7 week male CD-1 IGS mice (Charles River Laboratories) are administered 0.35 mg/kg of ceDNA vector construct 1-4 Luc (Group 1) in 1.2 mL volume via i.v. hydrodynamic administration on Day 0. Some animals in each group are re-administered an identical dose of ceDNA vector construct 1-4 Luc (Group 1) or on Day 28.

Expression of luciferase from ceDNA vector is assessed by in vivo chemiluminescence using IVIS® instrumentation following intraperitoneal (i.p.) injection of 150 mg/kg luciferase substrate. IVIS imaging is performed on Day 3, Day 4, Day 7, Day 14, Day 21, Day 28, Day 31, Day 35, and Day 42, and collected organs are imaged ex vivo following sacrifice on Day 42.

During the course of the study, animals are weighed and monitored daily for general health and well-being. At sacrifice, blood is collected from each animal by terminal cardiac stick, and split into two portions and processed to 1) plasma and 2) serum, with plasma snap-frozen and serum used for liver enzyme panel and subsequently snap frozen. Additionally, livers, spleens, kidneys, and inguinal lymph nodes (LNs) are collected and imaged ex vivo by IVIS.

Luciferase expression is assessed in livers by MAXDISCOVERY® Luciferase ELISA assay (BIOO Scientific/PerkinElmer), qPCR for Luciferase of liver samples, histopathology of liver samples and/or a serum liver enzyme panel (VetScanVS2; Abaxis Preventative Care Profile Plus).

### REFERENCES

All references listed and disclosed in the specification and Examples, including patents, patent applications, International patent applications and publications are incorporated herein in their entirety by reference.

What is claimed is:

1. A lipid particle comprising an ionizable lipid and a non-viral capsid-free DNA vector with covalently-closed ends (ceDNA vector), wherein the ceDNA vector comprises at least one heterologous nucleotide sequence operably positioned between asymmetric inverted terminal repeat sequences (asymmetric ITRs), wherein at least one of the asymmetric ITRs comprises a functional terminal resolution site and a Rep binding site.

2. The lipid nanoparticle of claim 1, wherein the ceDNA vector when digested with a restriction enzyme having a single recognition site on the ceDNA vector and analyzed by both native and denaturing gel electrophoresis displays characteristic bands of linear and continuous DNA as compared to linear and non-continuous DNA controls.

3. The lipid nanoparticle of claim for 2, wherein one or more of the asymmetric ITR sequences are from a virus selected from a parvovirus, a dependovirus, and an adeno-associated virus (AAV).

4. The lipid nanoparticle of claim 3, wherein the asymmetric ITRs are from different viral serotypes.

5. The lipid nanoparticle of of claim 4, wherein the one or more asymmetric ITRs are from an AAV serotype selected from AAV1, AAV2, AAV3, AAV4, AAV5, AAV6, AAV7, AAV8, AAV9, AAV10, AAV11, and AAV12.

6. The lipid nanoparticle of any one of claims 1-3, wherein one or more of the asymmetric ITR sequences are synthetic.

7. The lipid nanoparticle of any one of claims 1-6, wherein one or more of the ITRs is not a wild type ITR.

8. The lipid nanoparticle of any one of claims 1-7, wherein one or more both of the asymmetric ITRs is modified by a deletion, insertion, and/or substitution in at least one of the ITR regions selected from A, A′, B, B′, C, C′, D, and D′.

9. The lipid nanoparticle of any one of claims 1-8, wherein the ceDNA vector comprises at least two asymmetric ITRs selected from:
c. SEQ ID NO: 1 and SEQ ID NO:52; and
d. SEQ ID NO: 2 and SEQ ID NO: 51.

10. The lipid nanoparticle of any one of claims 1-9, wherein the ceDNA vector is obtained from a process comprising the steps of:
a. incubating a population of insect cells harboring a ceDNA expression construct in the presence of at least one Rep protein, wherein the ceDNA expression construct encodes the ceDNA vector, under conditions effective and for a time sufficient to induce production of the ceDNA vector within the insect cells; and
b. isolating the ceDNA vector from the insect cells.

11. The lipid nanoparticle of claim 10, wherein the ceDNA expression construct is selected from a ceDNA plasmid, a ceDNA bacmid, and a ceDNA baculovirus.

12. The lipid nanoparticle of claim 10 or claim 11, wherein the insect cell expresses at least one Rep protein.

13. The lipid nanoparticle of claim 10, wherein at least one Rep protein is from a virus selected from a parvovirus, a dependovirus, and an adeno-associated virus (AAV)

14. The lipid nanoparticle of claim 13, wherein at least one Rep protein is from an AAV serotype selected from AAV1, AAV2, AAV3, AAV4, AAV5, AAV6, AAV7, AAV8, AAV9, AAV10, AAV11, and AAV12.

15. The lipid particle of any one of claims 1-15, wherein the DNA vector is obtained from a vector polynucleotide, wherein the vector polynucleotide encodes a heterologous nucleic acid operatively positioned between two inverted terminal repeat sequences (ITRs), wherein the two ITS are different from each other (asymmetric), and at least one of the ITRs is a functional ITR comprising a functional terminal resolution site and a Rep binding site, and one of the ITRs comprises a deletion, insertion, and/or substitution relative to the functional ITR; the presence of Rep protein inducing replication of the vector polynucleotide and production of the DNA vector in an insect cell, the DNA vector being obtainable from a process comprising the steps of:
a. incubating a population of insect cells harboring the vector polynucleotide, which is devoid of viral capsid coding sequences, in the presence of Rep protein under conditions effective and for time sufficient to induce production of the capsid-free, non-viral DNA vector within the insect cells, wherein the insect cells do not comprise production of capsid-free, non-viral DNA within the insect cells in the absence of the vector; and
b. harvesting and isolating the capsid-free, non-viral DNA from the insect cells.

16. The lipid particle of any one of claims 10-15, wherein the presence of the capsid-free, non-viral DNA isolated from the insect cells can be confirmed.

17. The lipid particle of claim 16, wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.

18. The lipid particle of any one of claims 1-17, wherein the DNA vector is obtained from a vector polynucleotide, wherein the vector polynucleotide encodes a heterologous nucleic acid operatively positioned between a first and a second AAV2 inverted terminal repeat DNA polynucleotide sequence (ITRs), with at least one of the ITRs having at least one polynucleotide deletion, insertion, and/or substitution with respect to the corresponding AAV2 wild type ITR of SEQ ID NO:1 or SEQ ID NO:51 to induce replication of the DNA vector in an insect cell in the presence of Rep protein, the DNA vector being obtainable from a process comprising the steps of:
a. incubating a population of insect cells harboring the vector polynucleotide, which is devoid of viral capsid coding sequences, in the presence of Rep protein, under conditions effective and for a time sufficient to induce production of the capsid-free, non-viral DNA within the insect cells, wherein the insect cells do not comprise viral capsid coding sequences; and
b. harvesting and isolating the capsid-free, non-viral DNA from the insect cells.

19. The lipid particle of claim 18, wherein the presence of the capsid-free, non-viral DNA isolated from the insect cells can be confirmed.

20. The lipid particle of claim 19, wherein the presence of the capsid-free, non-viral DNA isolated from the insect cells can be confirmed by digesting DNA isolated from the insect cells with a restriction enzyme having a single recognition site on the DNA vector and analyzing the digested DNA material on a non-denaturing gel to confirm the presence of characteristic bands of linear and continuous DNA as compared to linear and non-continuous DNA.

21. The lipid particle of any one of claims 1-20, wherein the lipid particle further comprises one or more of a non-cationic lipid; a PEG conjugated lipid; and a sterol.

22. The lipid particle of any one of claims 1-21, wherein the ionizable lipid is a lipid described in Table 1.

23. The lipid particle of any one of claims 1-22, wherein the lipid particle further comprises a non-cationic lipid, wherein the non-ionic lipid is selected from the group consisting of distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanol amine (D SPE), monomethyl-phosphatidylethanolamine, dimethyl-phosphatidylethanolamine, 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, and dilinoleoylphosphatidylcholine.

24. The lipid particle of any one of claims 1-23, wherein the lipid particle further comprises a conjugated lipid, wherein the conjugated lipid, wherein the conjugated-lipid is selected from the group consisting of PEG-diacylglycerol (DAG), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG), PEG dialkoxypropylcarbam, and N-(carbonyl-methoxypolyethylene glycol 2000)-1,2-distearoyl-sn-glycero-3-phosphoethanolamine sodium salt.

25. The lipid particle of any one of claims 1-24, wherein the lipid particle further comprises cholesterol or a cholesterol derivative.

26. The lipid particle of any one of claims 1-25, wherein the lipid particle comprises:
(v) an ionizable lipid;
(vi) a non-cationic lipid;
(vii) a conjugated lipid that inhibits aggregation of particles; and
(viii) a sterol.

27. The lipid particle of any one of claims 1-26, wherein the lipid particle comprises:
(e) an ionizable lipid in an amount from about 20 mol % to about 90 mol % of the total lipid present in the particle;
(f) a non-cationic lipid in an amount from about 5 mol % to about 30 mol % of the total lipid present in the particle;
(g) a conjugated lipid that inhibits aggregation of particles in an amount from about 0.5 mol % to about 20 mol % of the total lipid present in the particle; and
(h) a sterol in an amount from about 20 mol % to about 50 mol % of the total lipid present in the particle.

28. The lipid particle of any one of claims 1-27, wherein total lipid to DNA vector (mass or weight) ratio is from about 10:1 to about 30:1.

29. A composition comprising a first lipid nanoparticle and an additional compound, wherein the first lipid nanoparticle comprises a first capsid free, non-viral vector, and is a lipid nanoparticle of any one of claims 1-28.

30. The composition of claim 29, wherein said additional compound is encompassed in a second lipid nanoparticle, and wherein the first and second lipid nanoparticles are different.

31. The composition of claim 28 or 29, wherein said additional compound is encompassed in the first lipid nanoparticle.

32. The composition of any one of claims 28-30, wherein said additional compound is a therapeutic agent.

33. The composition of claim 28, where said additional compound is a second capsid free, non-viral vector, wherein the first and second capsid free, non-viral vectors are different.
